(19) United States
(12) Patent Application Publication
(10) Pub. No.: US 2021/0275081 A1
(43) Pub. Date: Sep. 9, 2021

(54) APPARATUS AND METHOD FOR DISTINGUISHING WIDE COMPLEX HEART BEATS

(71) Applicant: Mayo Foundation for Medical Education and Research, Rochester, MN (US)

(72) Inventors: Adam M. May, Rochester, MN (US); Christopher V. Desimone, Rochester, MN (US); Abhishek J. Deshmukh, Rochester, MN (US)

(21) Appl. No.: 17/253,262

(22) PCT Filed: Aug. 15, 2019
(86) PCT No.: PCT/US2019/046713

§ 371 Date: Dec. 17, 2020

Related U.S. Application Data
(63) Continuation-in-part of application No. 16/445,036, filed on Jun. 18, 2019.
(60) Provisional application No. 62/870,266, filed on Jul. 3, 2019.
(60) Provisional application No. 62/688,265, filed on Jun. 21, 2018.
(60) Provisional application No. 62/688,265, filed on Jun. 21, 2018.

(51) Int. Cl. A61B 5/366; A61B 5/00; A61B 5/363; A61B 5/355; A61B 5/341; A61B 5/35; A61B 5/389; G16H 50/20; G16H 50/50; G16H 40/67

(52) U.S. Cl. CPC A61B 5/366; A61B 5/7264; A61B 5/363; A61B 5/355; A61B 5/341; A61B 2560/0487; A61B 5/389; G16H 50/20; G16H 50/50; G16H 40/67; A61B 5/35

(57) ABSTRACT

An apparatus and computerized method accurately distinguishes VT and SWCT without the need for manual ECG, electrogram (EMG) and/or vectorcardiogram (VCG) interpretation or calculation. The apparatus and computerized method provides three types of wide complex beat differentiation that can be automatically implemented using data provided by ECG, EMG and/or VCG interpretation software. The first type is based in whole or in part on a WCT Formula. The second type is based in whole or in part on a VT prediction model. The third type is based in whole or in part on an analysis of ventricular repolarization (e.g., T wave).

### CROSS-REFERENCE TO RELATED APPLICATIONS

This application claims priority to U.S. patent application Ser. No. 16/445,036 filed on Jun. 18, 2019 and entitled “Apparatus and Method for Differentiating Wide Complex Heart Beats”, which claims priority to U.S. Provisional Patent Application No. 62/688,265, filed on Jun. 21, 2018 and entitled “Apparatus and Method for Differentiating Wide Complex Heart, Beats”, and U.S. Provisional Patent Application No. 62/870,266, filed Jul. 3, 2019 and entitled “Apparatus and Method for Distinguishing Wide Complex Heart Beats” all of which are hereby incorporated by reference in its entirety.

### TECHNICAL FIELD OF THE INVENTION

The present invention relates in general to heart rhythms, and more particularly, to an apparatus and method for distinguishing wide complex heart beats.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with classifying wide complex tachycardia (WCT).

The successful differentiation of wide complex tachycardias (WCTs) into ventricular tachycardia (VT) or supraventricular wide complex tachycardia (SWCT) has undeniably important therapeutic and prognostic implications. Ventricular tachycardia (VT) is an abnormal rapid heart rhythm that is often dangerous. Supraventricular wide complex tachycardia (SWCT) is a similar appearing abnormal rapid heart rhythm that is typically less hazardous.

The 12-lead electrocardiogram (ECG) is the most practical test to non-invasively differentiate VT and SWCT, in part, because it is one of the most commonly used diagnostic tests performed in medicine (˜300 million ECGs are performed each year in the United states). Unfortunately, the differentiation of VT and SWCT remains problematic despite the availability of numerous manually-operated ECG criteria and algorithms (1-15). These manual interpretation methods do not perform well when used by less experienced ECG interpreters. In fact, few clinicians, aside from expert electrocardiographers, are able use manual methods with reliable accuracy. In addition, published conventional ECG interpretation methods are limited by their (1) compulsory need for manual ECG interpretation, (2) inability to estimate VT probability and (3) uncertain diagnostic performance when applied on WCTs regularly encountered in clinical practice.

### SUMMARY OF THE INVENTION

The present invention describes an ability to accurately distinguish VT and SWCT without the need for manual ECG, electrogram (EMG) and/or vectorcardiogram (VCG) interpretation or calculation. The present invention provides three types of embodiments for wide complex beat differentiation that can be automatically implemented using data provided by ECG, EMG and/or VCG interpretation software. The first type is based in whole or in part on a WCT Formula. The second type is based in whole or in part on a VT prediction model. The third type is based in whole or in part on an analysis of ventricular repolarization (e.g., T wave).

WCT Formula

One embodiment of the present invention provides a computerized method of classifying a wide complex heart beat(s) comprising: providing a computing device having an input/output interface, one or more processors and a memory; receiving one or more wide complex heart beat waveform amplitudes and/or time-voltage areas, and one or more baseline heart beat waveform amplitudes and/or time-voltage areas via the input/output interface or the memory; determining a signal change between the wide complex heart beat waveform amplitudes and/or time-voltage areas and the baseline heart beat waveform amplitudes and/or time-voltage areas using the one or more processors; and providing the signal change via the input/output interface, wherein the signal change provides an indication whether the wide complex heart beat(s) is from a ventricular source or a supraventricular aberrant condition.

In one aspect, the signal change further provides the indication whether the wide complex heart beat(s) is due to ventricular pacing. In another aspect, the wide complex heart beat(s) comprise a wide complex tachycardia (WCT), the ventricular source comprises a ventricular tachycardia (VT), and the supraventricular aberrant condition comprises a supraventricular wide complex tachycardia (SWCT). In another aspect, providing the signal change via the input/output interface comprises: automatically determining a wide complex heart beat classification for the wide complex heart beat(s) by comparing the signal change to a predetermined value using the one or more processors, wherein the wide complex heart beat classification comprises a ventricular source or a supraventricular aberrant condition; and providing the wide complex heart beat classification via the input/output interface. In another aspect, the signal change comprises a VT probability, the wide complex heart beat classification comprises a VT whenever the VT probability is greater than or equal to the predetermined value, and the wide complex heart beat classification comprises a SWCT whenever the VT probability is less than the predetermined value. In another aspect, the method further comprises selecting the predetermined value from a range of 0% to 100%. In another aspect, the predetermined value comprises about 1%, 10%, 25%, 50%, 75%, 90% or 99%. In another aspect, providing the signal change comprises providing a “shock” signal, a “no shock” signal, or no signal. In another aspect, the method further comprises obtaining the wide complex heart beat waveform amplitudes and/or time-voltage areas and the baseline heart beat waveform amplitudes and/or time-voltage areas from an electrocardiogram (ECG) QRS signal, a ventricular electrogram (EMG) signal, and/or a vectorcardiogram (VCG) signal. In another aspect, the wide complex heart beat waveform amplitudes and/or time-voltage areas comprise a plurality of measured amplitudes and/or time-voltage areas of a ECG QRS waveform, a EMG waveform and/or a VCG waveform above and below an isoelectric baseline; and the baseline heart beat waveform amplitudes and/or time-voltage areas comprise a plurality of measured amplitudes and/or time-voltage areas of a baseline ECG QRS waveform, a baseline EMG waveform and/or a baseline VCG waveform above and below the isoelectric baseline.

In another aspect, receiving the one or more wide complex heart beat waveform amplitudes and/or time-voltage areas, and one or more baseline heart beat waveform amplitudes and/or time-voltage areas comprises: receiving a ECG QRS data, a EMG data, a VCG data and/or a mathematically synthesized VCG data via the input/output interface or the memory; receiving a baseline ECG QRS data, a baseline EMG data and/or a baseline VCG data via the input/output interface or the memory; determining the one or more waveform amplitudes and/or time-voltage areas from the ECG QRS data, the EMG data and/or the VCG data using the one or more processors; and determining the one or more baseline waveform amplitudes and/or time-voltage areas from the baseline ECG QRS data, the baseline EMG data and/or the baseline VCG data using the one or more processors. In another aspect, the ECG QRS data, the EMG data and/or the VCG data is generated or recorded before or after the baseline ECG QRS data, the baseline EMG data and/or the baseline VCG data. In another aspect, the ECG QRS data, the EMG data and/or the VCG data is generated or recorded after the baseline ECG QRS data, the baseline EMG data and/or the baseline VCG data and determining the signal change. In another aspect, the method further comprises generating or recording the ECG QRS data, the EMG data and/or the VCG data and the baseline ECG QRS data, the baseline EMG data and/or the baseline VCG data using one or more sensors or devices. In another aspect, the one or more sensors or devices comprise a 12-lead ECG device, a continuous ECG telemetry monitor, a stress testing system, an extended monitoring device, a smartphone-enabled ECG medical device, an external cardioverter-defibrillator therapy device, a subcutaneous implantable cardioverter defibrillators (ICD), a pacemaker, an automated external defibrillators (AED), or an automatic implantable cardioverter defibrillator (AICD). In another aspect, the computing device is integrated into the one or more sensors or devices; or the one or more sensors or devices are integrated into the computing device. In another aspect, determining the signal change between the wide complex heart beat waveform amplitudes and/or time-voltage areas and the baseline heart beat waveform amplitudes and/or time-voltage areas comprises: receiving a wide complex heart beat waveform duration via the input/output interface or the memory; determining, using the one or more processors, a percent amplitude change (PAC) based on the wide complex heart beat waveform amplitudes and the baseline wide complex heart beat waveform amplitudes, and/or a percent time-voltage area change (PTVAC) based on the wide complex heart beat waveform time-voltage areas and the baseline wide complex heart beat waveform time-voltage areas; determining a classification probability based on the wide complex heart beat waveform duration, and the PAC and/or the PTVAC using the one or more processors; and wherein the signal change comprises the classification probability, and the classification probability comprises a VT probability, a SWCT probability, or a ventricular pacing probability. In another aspect, determining the classification probability is further determined based one or more additional classification predictors. In another aspect, the PAC comprises a frontal PAC and a horizontal PAC, and the PTVAC comprises a frontal PTVAC and a horizontal PTVAC.

In another aspect, determining the signal change between the wide complex heart beat waveform amplitudes and/or time-voltage areas and the baseline heart beat waveform amplitudes and/or time-voltage areas comprises: receiving a WCT QRS duration via the input/output interface or the memory; the one or more wide complex heart beat waveform amplitudes and/or time-voltage areas comprise one or more frontal plane WCT positive waveform amplitudes and/or time-voltage areas, one or more horizontal plane WCT positive waveform amplitudes and/or time-voltage areas, one or more frontal plane WCT negative waveform amplitudes and/or time-voltage areas, and one or more horizontal plane WCT negative waveform amplitudes and/or time-voltage areas; the one or more the baseline heart beat waveform amplitudes and/or time-voltage areas comprise one or more frontal plane baseline positive waveform amplitudes and/or time-voltage areas, one or more horizontal plane baseline positive waveform amplitudes and/or time-voltage areas, one or more frontal plane baseline negative waveform amplitudes and/or time-voltage areas, and one or more horizontal baseline negative waveform amplitudes and/or time-voltage areas; determining (1) a frontal percent amplitude change (PAC) based on the one or more frontal plane WCT positive waveform amplitudes, one or more frontal plane WCT negative waveform amplitudes, one or more frontal plane baseline positive waveform amplitudes, and one or more frontal plane baseline negative waveform amplitudes, and/or (2) a frontal percent time-voltage area (PTVAC) based on the one or more frontal plane WCT positive waveform time-voltage areas, one or more frontal plane WCT negative waveform time-voltage areas, one or more frontal plane baseline positive waveform time-voltage areas, and one or more frontal plane baseline negative waveform time-voltage areas; determining (1) a horizontal PAC based on the one or more horizontal plane WCT positive waveform amplitudes, one or more horizontal plane WCT negative waveform amplitudes, one or more horizontal plane baseline positive waveform amplitudes, and one or more horizontal baseline negative waveform amplitudes, and/or (2) a horizontal PTVAC based on the one or more horizontal plane WCT positive waveform time-voltage areas, one or more horizontal plane WCT negative waveform time-voltage areas, one or more horizontal plane baseline positive waveform time-voltage areas, and one or more horizontal baseline negative waveform time-voltage areas; determining a VT probability using a statistical or machine learning process based on the WCT QRS duration and (1) the frontal PAC and the horizontal PAC, and/or (2) the frontal PTVAC and the horizontal PTVAC; and wherein the signal change comprises the VT probability. In another aspect, the statistical or machine learning process comprises a linear regression algorithm, a logistic regression model, a linear discriminate analysis algorithm, a Naive Bayes algorithm, a computational model using artificial neural networks, a computational model based on classification or regression trees, a k-nearest neighbors based model, a support vector machine based model, a boosting algorithm, or an ensemble machine learning algorithm.

In another aspect, the frontal PAC is determined by

$$Frontal PAC ( \% ) = ( \frac{Frontal AAC}{Frontal BA} ) \times 100 ,$$

where: Frontal AAC=TAC_{aVR}+TAC_{aVL}+TAC_{aVF}, Frontal BA=TBA_{aVR}+TBA_{aVL}+TBA_{aVF}, TAC_{LeadX}=APC_{LeadX}+ANC_{LeadX}, TBA_{Baseline:LeadX}=(−)Amplitude_{Baseline:LeadX}+(+)Amplitude_{Baseline:LeadX}, APC_{LeadX}=|(+)Amplitude_{WCT:LeadX}−(+)AMplitude_{Baseline:LeadX}|, ANC_{LeadX}=|(−)Amplitude_{WCT:LeadX}−(−)Amplitude_{Baseline:LeadX}|, LeadX denotes V1, V4, V6 (horizontal plane) or aVL, aVR, aVF (frontal plane); the horizontal PAC is determined by

$$Horizontal PAC ( \% ) = ( \frac{Horizontal AAC}{Horizontal BA} ) \times 100 ,$$

where: Horizontal AAC=TAC_{V1}+TAC_{V4}+TAC_{V6}, Horizontal BA=TBA_{V1}+TBA_{V4}+TBA_{V6}; and the VT probability (P_{VT}) is determined by:

$${P}_{V T} = \frac{{e}^{( a + b \times {WCT}_{duration} + c \times {PAC}_{frontal} + d \times PA {C}_{horizontal} )}}{1 + {e}^{( a + b \times {WCT}_{duration} + c \times {PAC}_{frontal} + d \times PA {C}_{horizontal} )}} ,$$

where a, b, c and d are constants. In another aspect, the frontal PTVAC is determined by

$$Frontal PTVAC ( \% ) = ( \frac{Frontal ATVAC}{Frontal BTVA} ) \times 100 ,$$

where: Frontal ATVAC=TTVAC_{aVR}+TTVAC_{aVL}+TTVAC_{aVF}, Frontal BTVA=TBTVA_{aVR}+TBTVA_{aVL}, +TBTVA_{aVF}, TTVAC_{LeadX}=TVAPC_{LeadX}+TVANC_{LeadX}, TBTVA_{Baseline:LeadX}=(−)TimeVoltageArea_{Baseline:LeadX}+(+)TimeVoltageArea_{Baseline:LeadX}, TVAPC_{leadX}=|(+)TimeVoltageArea_{WCT:LeadX} (+)TimeVoltageArea_{Baseline:LeadX}|, TVANC_{leadX}=|(−)TimeVoltageArea_{WCT:LeadX}−(−)TimeVoltageArea_{Baseline:LeadX}|,
LeadX denotes V1, V4, V6 (horizontal plane) or aVL, aVR, aVF (frontal plane); the horizontal PTVAC is determined by

$$Horizontal PTVAC ( \% ) = ( \frac{Horizontal ATVAC}{Horizontal BTVA} ) \times 100 ,$$

where: Horizontal ATVAC=TTVAC_{V1}+TTVAC_{V4}+TTVAC_{V6},

Horizontal BTVA=TBTVA_{V1}+TBTVA_{V4}+TBTVA_{V6}; and the VT probability (P_{VT}) is determined by:

$${P}_{V T} = \frac{{e}^{( a + b \times {WCT}_{duration} + c \times {PTVAC}_{frontal} + d \times {PTVAC}_{horizontal} )}}{1 + {e}^{( a + b \times {WCT}_{duration} + c \times {PTVAC}_{frontal} + d \times {PTVAC}_{horizontal} )}} ,$$

where: a, b, c and d are constants.

In another aspect, the input/output interface comprises a remote device, and the remote device is communicably coupled to the one or more processors via one or more networks. In another aspect, the method further comprises providing a recommendation to select or exclude a therapy, medication, diagnostic testing or referral for a patient based on the signal change. In another aspect, the computing device comprises a server computer, a workstation computer, a laptop computer, a mobile communications device, a personal data assistant, or a medical device. Moreover, the method can be implemented using a non-transitory computer readable medium that when executed causes the one or more processors to perform the method.

Another embodiment of the present invention provides an apparatus for classifying a wide complex heart beat(s) comprising an input/output interface, a memory, and one or more processors communicably coupled to the input/output interface and the memory. The one or more processors: receive one or more wide complex heart beat waveform amplitudes and/or time-voltage areas, and one or more baseline heart beat waveform amplitudes and/or time-voltage areas via the input/output interface or the memory, determine a signal change between the wide complex heart beat waveform amplitudes and/or time-voltage areas and the baseline heart beat waveform amplitudes and/or time-voltage areas using the one or more processors, and provide the signal change via the input/output interface, wherein the signal change provides an indication whether the wide complex heart beat(s) is from a ventricular source or a supraventricular aberrant condition.

In one aspect, the signal change further provides the indication whether the wide complex heart beat(s) is due to ventricular pacing. In another aspect, the wide complex heart beat(s) comprise a wide complex tachycardia (WCT), the ventricular source comprises a ventricular tachycardia (VT), and the supraventricular aberrant condition comprises a supraventricular wide complex tachycardia (SWCT). In another aspect, the one or more processors provide the signal change via the input/output interface by: automatically determining a wide complex heart beat classification for the wide complex heart beat(s) by comparing the signal change to a predetermined value, wherein the wide complex heart beat classification comprises the ventricular source or the supraventricular aberrant condition; and providing the wide complex heart beat classification via the input/output interface. In another aspect, the signal change comprises a VT probability, the wide complex heart beat classification comprises a VT whenever the VT probability is greater than or equal to the predetermined value, and the wide complex heart beat classification comprises a SWCT whenever the VT probability is less than the predetermined value. In another aspect, the one or more processors select the predetermined value from a range of 0% to 100%. In another aspect, the predetermined value comprises about 1%, 10%, 25%, 50%, 75%, 90% or 99%. In another aspect, the one or more processors provide the signal change by providing a “shock” signal, a “no shock” signal, or no signal. In another aspect, the wide complex heart beat waveform amplitudes and/or time-voltage areas and the baseline heart beat waveform amplitudes and/or time-voltage areas are obtained from an electrocardiogram (ECG) QRS signal, a ventricular electrogram (EMG) signal, and/or a vectorcardiogram (VCG) signal. In another aspect, the wide complex heart beat waveform amplitudes and/or time-voltage areas comprise a plurality of measured amplitudes and/or time-voltage areas of a ECG QRS waveform, a EMG waveform and/or a VCG waveform above and below an isoelectric baseline; and the baseline heart beat waveform amplitudes and/or time-voltage areas comprise a plurality of measured amplitudes and/or time-voltage areas of a baseline ECG QRS waveform, a baseline EMG waveform and/or a baseline VCG waveform above and below the isoelectric baseline.

In another aspect, the one or more processors receive the one or more wide complex heart beat waveform amplitudes and/or time-voltage areas, and one or more baseline heart beat waveform amplitudes and/or time-voltage areas by: receiving a ECG QRS data, a EMG data, a VCG data and/or a mathematically synthesized VCG data via the input/output interface or the memory; receiving a baseline ECG QRS data, a baseline EMG data and/or a baseline VCG data via the input/output interface or the memory; determining the one or more waveform amplitudes and/or time-voltage areas from the ECG QRS data, the EMG data and/or the VCG data; and determining the one or more baseline waveform amplitudes and/or time-voltage areas from the baseline ECG QRS data, the baseline EMG data and/or the baseline VCG data. In another aspect, the ECG QRS data, the EMG data and/or the VCG data is generated or recorded before or after the baseline ECG QRS data, the baseline EMG data and/or the baseline VCG data. In another aspect, the ECG QRS data, the EMG data and/or the VCG data is generated or recorded after the baseline ECG QRS data, the baseline EMG data and/or the baseline VCG data and determining the signal change. In another aspect, the ECG QRS data, the EMG data and/or the VCG data and the baseline ECG QRS data, the baseline EMG data and/or the baseline VCG data are generated or recorded using one or more sensors or devices. In another aspect, the one or more sensors or devices comprise a 12-lead ECG device, a continuous ECG telemetry monitor, a stress testing system, an extended monitoring device, a smartphone-enabled ECG medical device, a cardioverter-defibrillator therapy device, a subcutaneous implantable cardioverter defibrillators (ICD), a pacemaker, an automated external defibrillators (AED), or an automatic implantable cardioverter defibrillator (AICD). In another aspect, the input/output interface, the memory and the one or more processors are integrated into the one or more sensors or devices; or the one or more sensors or devices are integrated into a computing device comprising the input/output interface, the memory and the one or more processors. In another aspect, the one or more processors determine the signal change between the wide complex heart beat waveform amplitudes and/or time-voltage areas and the baseline heart beat waveform amplitudes and/or time-voltage areas by: receiving a wide complex heart beat waveform duration via the input/output interface or the memory; determining a percent amplitude change (PAC) based on the wide complex heart beat waveform amplitudes and the baseline wide complex heart beat waveform amplitudes, and/or a percent time-voltage area change (PTVAC) based on the wide complex heart beat waveform time-voltage areas and the baseline wide complex heart beat waveform time-voltage areas; determining a classification probability based on the wide complex heart beat waveform duration, the PAC and/or the PTVAC; and wherein the signal change comprises the classification probability, and the classification probability comprises a VT probability, a SWCT probability, or a ventricular pacing probability. In another aspect, determining the classification probability is further determined based one or more additional classification predictors. In another aspect, the PAC comprises a frontal PAC and a horizontal PAC, and the PTVAC comprises a frontal PTVAC and a horizontal PTVAC.

In another aspect, the one or more processors determine the signal change between the wide complex heart beat waveform amplitudes and/or time-voltage areas and the baseline heart beat waveform amplitudes and/or time-voltage areas by: receiving a WCT QRS duration via the input/output interface or the memory; the one or more wide complex heart beat waveform amplitudes and/or time-voltage areas comprise one or more frontal plane WCT positive waveform amplitudes and/or time-voltage areas, one or more horizontal plane WCT positive waveform amplitudes and/or time-voltage areas, one or more frontal plane WCT negative waveform amplitudes and/or time-voltage areas, and one or more horizontal plane WCT negative waveform amplitudes and/or time-voltage areas; the one or more the baseline heart beat waveform amplitudes and/or time-voltage areas comprise one or more frontal plane baseline positive waveform amplitudes and/or time-voltage areas, one or more horizontal plane baseline positive waveform amplitudes and/or time-voltage areas, one or more frontal plane baseline negative waveform amplitudes and/or time-voltage areas, and one or more horizontal baseline negative waveform amplitudes and/or time-voltage areas; determining (1) a frontal percent amplitude change (PAC) based on the one or more frontal plane WCT positive waveform amplitudes, one or more frontal plane WCT negative waveform amplitudes, one or more frontal plane baseline positive waveform amplitudes, and one or more frontal plane baseline negative waveform amplitudes, and/or (2) a frontal percent time-voltage area (PTVAC) based on the one or more frontal plane WCT positive waveform time-voltage areas, one or more frontal plane WCT negative waveform time-voltage areas, one or more frontal plane baseline positive waveform time-voltage areas, and one or more frontal plane baseline negative waveform time-voltage areas; determining (1) a horizontal PAC based on the one or more horizontal plane WCT positive waveform amplitudes, one or more horizontal plane WCT negative waveform amplitudes, one or more horizontal plane baseline positive waveform amplitudes, and one or more horizontal baseline negative waveform amplitudes, and/or (2) a horizontal PTVAC based on the one or more horizontal plane WCT positive waveform time-voltage areas, one or more horizontal plane WCT negative waveform time-voltage areas, one or more horizontal plane baseline positive waveform time-voltage areas, and one or more horizontal baseline negative waveform time-voltage areas; determining a VT probability using a statistical or machine learning process based on the WCT QRS duration and (1) the frontal PAC and the horizontal PAC, and/or (2) the frontal PTVAC and the horizontal PTVAC; and wherein the signal change comprises the VT probability. In another aspect, the statistical or machine learning process comprises a linear regression algorithm, a logistic regression model, a linear discriminate analysis algorithm, a Naive Bayes algorithm, a computational model using artificial neural networks, a computational model based on classification or regression trees, a k-nearest neighbors based model, a support vector machine based model, a boosting algorithm, or an ensemble machine learning algorithm.

In another aspect, the frontal PAC is determined by

$$Frontal PAC ( \% ) = ( \frac{Frontal AAC}{Frontal BA} ) \times 100 ,$$

where: Frontal AAC=TAC_{aVR}+TAC_{aVL}+TAC_{aVF}, Frontal BA=TBA_{aVR}+TBA_{aVL}+TBA_{aVF}, TAC_{LeadX}=APC_{LeadX}+ANC_{LeadX}, TBA_{Baseline:LeadX}=(−)Amplitude_{Baseline:LeadX}+(+)Amplitude_{Baseline:LeadX}, APC_{LeadX}=|(+)Amplitude_{WCT:LeadX}−(+)Amplitude_{Baseline:LeadX}|, ANC_{LeadX}=|(−)Amplitude_{WCT:LeadX}−(−)Amplitude_{Baseline:LeadX}|, LeadX denotes V1, V4, V6 (horizontal plane) or aVL, aVR, aVF (frontal plane); the horizontal PAC is determined by

$$Horizontal PAC ( \% ) = ( \frac{Horizontal AAC}{Horizontal BA} ) \times 100 ,$$

where: Horizontal AAC=TAC_{V1}+TAC_{V4}+TAC_{V6}, Horizontal BA=TBA_{V1}+TBA_{V4}+TBA_{V6}; and the VT probability (P_{VT}) is determined by:

$${P}_{V T} = \frac{{e}^{( a + b \times {WCT}_{duration} + c \times {PAC}_{frontal} + d \times P A {C}_{horizontal} )}}{1 + {e}^{( a + b \times {WCT}_{duration} + c \times {PAC}_{frontal} + d \times P A {C}_{horizontal} )}} ,$$

where a, b, c and d are constants. In another aspect, the frontal PTVAC is determined by

$$Frontal PTVAC ( \% ) = ( \frac{Frontal ATVAC}{Frontal BTVA} ) \times 100 ,$$

where: Frontal ATVAC=TTVAC_{aVR}+TTVAC_{aVL}+TTVAC_{aVF}, Frontal BTVA=TBTVA_{aVR}+TBTVA_{aVL}+TBTVA_{aVF}, TTVAC_{LeadX}=TVAPC_{LeadX}+TVANC_{LeadX}, TBTVA_{Baseline:LeadX}=(−)TimeVoltageArea_{Baseline:LeadX} (+)TimeVoltageArea_{Baseline:LeadX}, TVAPC_{leadX}=|(+)TimeVoltageArea_{WCT:LeadX}−(+)TimeVoltageArea_{Baseline:LeadX}|, TVANC_{leadX}=|(−)TimeVoltageArea_{WCT:LeadX}−(−)TimeVoltageArea_{Baseline:LeadX}|,
LeadX denotes V1, V4, V6 (horizontal plane) or aVL, aVR, aVF (frontal plane); the horizontal PTVAC is determined by

$$Horizontal PTVAC ( \% ) = ( \frac{Horizontal ATVAC}{Horizontal BTVA} ) \times 100 ,$$

where: Horizontal ATVAC=TTVAC_{V1}+TTVAC_{V4}+TTVAC_{V6}

Horizontal BTVA=TBTVA_{V1}+TBTVA_{V4}+TBTVA_{V6}; and the VT probability (P_{VT}) is determined by:

$${P}_{V T} = \frac{{e}^{( a + b \times {WCT}_{duration} + c \times {PTVAC}_{frontal} + d \times {PTVAC}_{horizontal} )}}{1 + {e}^{( a + b \times {WCT}_{duration} + c \times {PTVAC}_{frontal} + d \times {PTVAC}_{horizontal} )}} ,$$

where: a, b, c and d are constants.

In another aspect, the input/output interface comprises a remote device, and the remote device is communicably coupled to the one or more processors via one or more networks. In another aspect, the one or more processors provide a recommendation to select or exclude a therapy, medication, diagnostic testing or referral for a patient based on the signal change. In another aspect, apparatus comprises a server computer, a workstation computer, a laptop computer, a mobile communications device, a personal data assistant, or a medical device.

VT Prediction Model

One embodiment of the present invention provides a computerized method of classifying a wide complex heart beat(s) comprising: providing a computing device having an input/output interface, one or more processors and a memory; receiving a wide complex heart beat data comprising at least a wide complex heart beat QRS duration, a wide complex heart beat R wave axis and a wide complex heart beat T wave axis via the input/output interface or the memory; receiving a baseline heart beat data comprising at least a baseline heart beat QRS duration, a baseline heart beat R wave axis, and a baseline heart beat T wave axis via the input/output interface or the memory; determining a signal change between the wide complex heart beat data and the baseline heart beat data areas using the one or more processors; and providing the signal change via the input/output interface, wherein the signal change provides an indication whether the wide complex heart beat(s) is from a ventricular source or a supraventricular source.

In one aspect, the signal change further provides the indication whether the wide complex heart beat(s) is due to ventricular pacing or a premature ventricular contraction (PVC). In another aspect, the wide complex heart beat(s) comprise a wide complex tachycardia (WCT); the ventricular source comprises a ventricular tachycardia (VT); and the supraventricular source comprises a supraventricular wide complex tachycardia (SWCT). In another aspect, providing the signal change via the input/output interface comprises: automatically determining a wide complex heart beat classification for the wide complex heart beat(s) by comparing the signal change to a predetermined value using the one or more processors, wherein the wide complex heart beat classification comprises the ventricular source or the supraventricular source; and providing the wide complex heart beat classification via the input/output interface. In another aspect, the signal change comprises a VT probability; the wide complex heart beat classification comprises a VT whenever the VT probability is greater than or equal to the predetermined value; and the wide complex heart beat classification comprises a SWCT whenever the VT probability is less than the predetermined value. In another aspect, the method further comprises selecting the predetermined value from a range of 0% to 100%. In another aspect, the predetermined value comprises about 1%, 10%, 25%, 50%, 75%, 90% or 99%. In another aspect, providing the signal change comprises providing a “shock” recommendation signal, a “no shock” recommendation signal, or no signal. In another aspect, the method further comprises obtaining the wide complex heart beat data and the baseline heart beat waveform data from an electrocardiogram (ECG) QRS signal, a ventricular electrogram (EMG) signal, vectorcardiogram (VCG) signal and/or a mathematically-synthesized VCG signal. In another aspect, receiving the wide complex heart beat data comprises receiving a ECG QRS data, a EMG data, a VCG data and/or a mathematically-synthesized VCG data via the input/output interface or the memory, and determining the wide complex heart beat data from the ECG QRS data, the EMG data, the VCG data and/or the mathematically-synthesized VCG data using the one or more processors; and receiving the baseline heart beat data comprises receiving a baseline ECG QRS data, a baseline EMG data, a baseline VCG data, and/or a baseline mathematically-synthesized VCG data via the input/output interface or the memory, and determining the baseline heart beat data from the ECG QRS data, the EMG data, the VCG data, and/or the mathematically-synthesized VCG data using the one or more processors. In another aspect, the ECG QRS data, the EMG data, the VCG data and/or the mathematically-synthesized VCG data, is generated or recorded before or after the baseline ECG QRS data, the baseline EMG data, the baseline VCG data and/or the mathematically-synthesized VCG data. In another aspect, the ECG QRS data, the EMG data, the VCG data, and/or mathematically-synthesized VCG data is generated or recorded after the baseline ECG QRS data, the baseline EMG data, the baseline VCG data, and/or the baseline mathematically-synthesized VCG data and determining the signal change.

In another aspect, the method further comprises generating or recording the ECG QRS data, the EMG data, the VCG data, and the mathematically-synthesized VCG data as well as the baseline ECG QRS data, the baseline EMG data, the baseline VCG data, and/or the baseline mathematically-synthesized VCG data using one or more sensors or devices. In another aspect, the one or more sensors or devices comprise two or more leads of a ECG device, a 12-lead ECG device, a continuous ECG telemetry monitor, a stress testing system, an extended monitoring device, a smartphone-enabled ECG medical device, a cardioverter-defibrillator therapy device, a subcutaneous implantable cardioverter defibrillators (ICD), an intracardiac or transvenous pacemaker device, an automated external defibrillators (AED), or an automatic implantable cardioverter defibrillator (AICD). In another aspect, the computing device is integrated into the one or more sensors or devices; or the one or more sensors or devices are integrated into the computing device. In another aspect, the signal change comprises a classification probability comprising a VT probability, a SWCT probability, a premature ventricular contraction probability, or a ventricular pacing probability. In another aspect determining the classification probability is further determined based one or more additional classification predictors. In another aspect, determining the signal change comprises determining a VT probability using a statistical or machine learning process. In another aspect, the statistical or machine learning process comprises a linear regression algorithm, a logistic regression model, a linear discriminate analysis algorithm, a Naive Bayes algorithm, a computational model using artificial neural networks, a computational model based on classification or regression trees, a k-nearest neighbors based model, a support vector machine based model, a boosting algorithm, or an ensemble machine learning algorithm. In another aspect, the signal change comprises determining a VT probability (P_{VT}) by:

$${P}_{VT} = \frac{{e}^{( a + b \times ( R axis change ) + c \times ( T axis change ) + d \times ( WCT QRS duration ) + g \times ( QRS duration change ) )}}{\begin{matrix}1 + \\ {e}^{( a + b \times ( R axis change ) + c \times ( T axis change ) + d \times ( WCT QRS duration ) + g \times ( QRS duration change ) )}\end{matrix}} ,$$

where: a, b, c, d and g are constants,
the QRS axis change=an absolute or non-absolute value of the wide complex heart beat R wave axis minus the baseline heart beat R wave axis,
the T axis change=an absolute or non-absolute value of the wide complex heart beat T wave axis minus the baseline heart beat T wave axis,
the WCT QRS duration=the wide complex heart beat QRS duration,
the QRS duration change=an absolute or non-absolute value of the wide complex heart beat QRS duration minus the baseline heart beat QRS duration.

In another aspect, the input/output interface comprises a remote device, and the remote device is communicably coupled to the one or more processors via one or more networks. In another aspect, receiving the wide complex heart beat data comprises monitoring a person using one or more sensors or devices communicably coupled to the input/output interface. In another aspect, the method further comprises providing a recommendation to select or exclude a therapy, medication, diagnostic testing or referral for a patient based on the signal change. In another aspect, the method further comprises sending an alert to one or more devices in based on the signal change. In another aspect, the method further comprises counting the frequency of various wide complex beats comprising ventricular tachycardia events, supraventricular tachycardia events, singular supraventricular wide complex beats, premature ventricular contractions, right ventricular pacing events and/or biventricular pacing events. In another aspect, the method further comprises: receiving multiple sets of the wide complex heart beat data and the baseline heart beat data for a person or group of persons; determining the signal change for each set of the wide complex heart beat data and the baseline heart beat data for the person or the group of persons; and creating a VT prediction model for the person or the group of persons using the signal changes. In another aspect, the computing device comprises a server computer, a workstation computer, a laptop computer, a mobile communications device, a personal data assistant, or a medical device.

Another embodiment of the present invention provides an apparatus for classifying a wide complex heart beat(s) comprising an input/output interface, a memory, and one or more processors communicably coupled to the input/output interface and the memory. The one or more processors: receive a wide complex heart beat data comprising at least a wide complex heart beat QRS duration, a wide complex heart beat R wave axis and a wide complex heart beat T wave axis via the input/output interface or the memory; receive a baseline heart beat data comprising at least a baseline heart beat QRS duration, a baseline heart beat R wave axis, and a baseline heart beat T wave axis via the input/output interface or the memory; determine a signal change between the wide complex heart beat data and the baseline heart beat data areas; and provide the signal change via the input/output interface, wherein the signal change provides an indication whether the wide complex heart beat(s) is from a ventricular source or a supraventricular source.

In one aspect, the signal change further provides the indication whether the wide complex heart beat(s) is due to ventricular pacing or a premature ventricular contraction (PVC). In another aspect, the wide complex heart beat(s) comprise a wide complex tachycardia (WCT); the ventricular source comprises a ventricular tachycardia (VT); and the supraventricular source comprises a supraventricular wide complex tachycardia (SWCT). In another aspect, the one or more processors provide the signal change via the input/output interface by: automatically determining a wide complex heart beat classification for the wide complex heart beat(s) by comparing the signal change to a predetermined value wherein the wide complex heart beat classification comprises the ventricular source or the supraventricular source; and providing the wide complex heart beat classification via the input/output interface. In another aspect, the signal change comprises a VT probability; the wide complex heart beat classification comprises a VT whenever the VT probability is greater than or equal to the predetermined value; and the wide complex heart beat classification comprises a SWCT whenever the VT probability is less than the predetermined value. In another aspect, the one or more processors select the predetermined value from a range of 0% to 100%. In another aspect, the predetermined value comprises about 1%, 10%, 25%, 50%, 75%, 90% or 99%. In another aspect, the one or more processors provide the signal change by providing a “shock” recommendation signal, a “no shock” recommendation signal, or no signal. In another aspect, the one or more processors obtain the wide complex heart beat data and the baseline heart beat waveform data from an electrocardiogram (ECG) QRS signal, a ventricular electrogram (EMG) signal, vectorcardiogram (VCG) signal and/or mathematically-synthesized (VCG) signal. In another aspect, the one or more processors: receive the wide complex heart beat data by receiving a ECG QRS data, a EMG data, a VCG data and/or a mathematically-synthesized VCG data via the input/output interface or the memory, and determine the wide complex heart beat data from the ECG QRS data, the EMG data, the VCG data and/or the mathematically-synthesized VCG data; and receive the baseline heart beat data by receiving a baseline ECG QRS data, a baseline EMG data, a baseline VCG data, and/or a mathematically-synthesized VCG data via the input/output interface or the memory, and determine the baseline heart beat data from the ECG QRS data, the EMG data, the VCG data and/or the mathematically-synthesized VCG data. In another aspect, the ECG QRS data, the EMG data, the VCG data and/or the mathematically-synthesized VCG data is generated or recorded before or after the baseline ECG QRS data, the baseline EMG data, the baseline VCG data and/or the mathematically-synthesized VCG data. In another aspect, the ECG QRS data, the EMG data, the VCG data, and/or the mathematically-synthesized VCG data is generated or recorded after the baseline ECG QRS data, the baseline EMG data, the baseline VCG data, and/or the baseline mathematically-synthesized VCG data, and determining the signal change.

In another aspect, the one or more processors generate or record the ECG QRS data, the EMG data, the VCG data, and/or the mathematically-synthesized VCG data and the baseline ECG QRS data, the baseline EMG data and/or the baseline VCG data using one or more sensors or devices. In another aspect, the one or more sensors or devices comprise two or more leads of a ECG device, a 12-lead ECG device, a continuous ECG telemetry monitor, a stress testing system, an extended monitoring device, a smartphone-enabled ECG medical device, a cardioverter-defibrillator therapy device, a subcutaneous implantable cardioverter defibrillators (ICD), an intracardiac or transvenous pacemaker device, an automated external defibrillators (AED), or an automatic implantable cardioverter defibrillator (AICD). In another aspect, the computing device is integrated into the one or more sensors or devices; or the one or more sensors or devices are integrated into the computing device. In another aspect, the signal change comprises a classification probability comprising a VT probability, a SWCT probability, a premature contraction probability, or a ventricular pacing probability. In another aspect, the one or more processors determine the classification probability based one or more additional classification predictors. In another aspect, the one or more processors determine the signal change by determining a VT probability using a statistical or machine learning process. In another aspect, the statistical or machine learning process comprises a linear regression algorithm, a logistic regression model, a linear discriminate analysis algorithm, a Naive Bayes algorithm, a computational model using artificial neural networks, a computational model based on classification or regression trees, a k-nearest neighbors based model, a support vector machine based model, a boosting algorithm, or an ensemble machine learning algorithm. In another aspect, the one or more processors determine the signal change by determining a VT probability (P_{VT}) by:

$${P}_{VT} = \frac{{e}^{( a + b \times ( R axis change ) + c \times ( T axis change ) + d \times ( WCT QRS duration ) + g \times ( QRS duration change ) )}}{\begin{matrix}1 + \\ {e}^{( a + b \times ( R axis change ) + c \times ( T axis change ) + d \times ( WCT QRS duration ) + g \times ( QRS duration change ) )}\end{matrix}} ,$$

where: a, b, c, d and g are constants,
the QRS axis change=an absolute or non-absolute value of the wide complex heart beat R wave axis minus the baseline heart beat R wave axis,
the T axis change=an absolute or non-absolute value of the wide complex heart beat T wave axis minus the baseline heart beat T wave axis,
the WCT QRS duration=the wide complex heart beat QRS duration,
the QRS duration change=an absolute or non-absolute value of the wide complex heart beat QRS duration minus the baseline heart beat QRS duration.

In another aspect, the input/output interface comprises a remote device, and the remote device is communicably coupled to the one or more processors via one or more networks. In another aspect, the one or more processors receive the wide complex heart beat data by monitoring a person using one or more sensors or devices communicably coupled to the input/output interface. In another aspect, the one or more processors provide a recommendation to select or exclude a therapy, medication, diagnostic testing or referral for a patient based on the signal change. In another aspect, the one or more processors send an alert to one or more devices in based on the signal change. In another aspect, the one or more processors count the frequency of various wide complex beats comprising ventricular tachycardia events, supraventricular tachycardia events, singular supraventricular wide complex beats, premature ventricular contraction, right ventricular pacing and/or biventricular pacing. In another aspect, the one or more processors receive multiple sets of the wide complex heart beat data and the baseline heart beat data for a person or group of persons, determine the signal change for each set of the wide complex heart beat data and the baseline heart beat data for the person or the group of persons, and create a VT prediction model for the person or the group of persons using the signal changes. In another aspect, the computing device comprises a server computer, a workstation computer, a laptop computer, a mobile communications device, a personal data assistant, or a medical device.

Ventricular Repolarization

One embodiment of the present invention provides a computerized method of classifying a wide complex heart beat(s) comprising: providing a computing device having an input/output interface, one or more processors and a memory; receiving one or more wide complex heart beat waveform amplitudes and/or time-voltage areas of ventricular repolarization, and one or more baseline heart beat waveform amplitudes and/or time-voltage areas of ventricular repolarization via the input/output interface or the memory; determining a signal change in ventricular repolarization between the wide complex heart beat waveform amplitudes and/or time-voltage areas of ventricular repolarization and the baseline heart beat waveform amplitudes and/or time-voltage areas of ventricular repolarization using the one or more processors; and providing the signal change in ventricular repolarization via the input/output interface, wherein the signal change in ventricular repolarization provides an indication whether the wide complex heart beat(s) is from a ventricular source or a supraventricular source.

In one aspect, the signal changes in the ventricular repolarization further provides the indication whether the wide complex heart beat(s) is due to ventricular pacing or a premature ventricular contraction (PVC). In another aspect, the wide complex heart beat(s) comprise a wide complex tachycardia (WCT), the ventricular source comprises a ventricular tachycardia (VT), and the supraventricular source comprises a supraventricular wide complex tachycardia (SWCT). In another aspect, providing the signal changes in the ventricular repolarization via the input/output interface comprises: automatically determining a wide complex heart beat classification for the wide complex heart beat(s) by comparing the signal change to a predetermined value using the one or more processors, wherein the wide complex heart beat classification comprises a ventricular source or a supraventricular source; and providing the wide complex heart beat classification via the input/output interface. In another aspect, the signal change comprises a VT probability, the wide complex heart beat classification comprises a VT whenever the VT probability is greater than or equal to the predetermined value, and the wide complex heart beat classification comprises a SWCT whenever the VT probability is less than the predetermined values. In another aspect, the method further comprises selecting the predetermined value from a range of 0% to 100%. In another aspect, the predetermined value comprises about 1%, 10%, 25%, 50%, 75%, 90% or 99%. In another aspect, providing the signal changes in ventricular repolarization comprises providing a “shock” recommendation signal, a “no shock” recommendation signal, or no signal. In another aspect, the method further comprises obtaining wide complex beat T-wave amplitudes and/or time-voltage areas and the baseline T-wave amplitudes and/or time-voltage areas from an electrocardiogram (ECG) signal, a ventricular electrogram (EMG) signal, a vectorcardiogram (VCG) signal and/or mathematically-synthesized vectorcardiogram (VCG) signal. In another aspect, the T-wave amplitudes and/or time-voltage areas comprise a plurality of measured T-wave amplitudes and/or time-voltage areas of a ECG waveform, a EMG waveform, a VCG waveform, and/or a mathematically-synthesized vectorcardiogram (VCG) waveform above and below the isometric baseline.

In another aspect, receiving the one or more wide complex beat T-wave amplitudes and/or time-voltage areas, and one or more baseline T-wave amplitudes and/or time-voltage areas comprises: receiving a ECG data, a EMG data, a VCG data, or a mathematically-synthesized VCG data via the input/output interface or the memory; receiving a baseline ECG data, a baseline EMG data, a baseline VCG data, or a baseline mathematically-synthesized VCG data via the input/output interface or the memory; determining the one or more T-wave amplitudes and/or time-voltage areas from the ECG data, the EMG data, the VCG data, and/or the mathematically-synthesized VCG data using one or more processors; and determining the one or more baseline waveform amplitudes and/or time-voltage areas from the baseline ECG data, the baseline EMG data, the baseline VCG data, and/or the mathematically-synthesized VCG data using the one or more processors. In another aspect, the ECG data, the EMG data, the VCG data, or the mathematically-synthesized VCG data is generated or recorded before or after the baseline ECG data, the baseline EMG data, the baseline VCG data, and/or the baseline mathematically-synthesized VCG data. In another aspect, the ECG data, the EMG data, the VCG data, and/or the mathematically-synthesized VCG data is generated or recorded after the baseline ECG data, the baseline EMG data, the baseline VCG data, and/or the baseline mathematically-synthesized VCG data and determining the changes in ventricular repolarization (i.e., T-wave changes). In another aspect, the method further comprises generating or recording the ECG data, the EMG data, the VCG data, and/or the mathematically-synthesized VCG data and the baseline ECG data, the baseline EMG data, the baseline VCG data, and/or the baseline mathematically-synthesized VCG data using one or more sensors or devices. In another aspect, the one or more sensors or devices comprise a 12-lead ECG device, a continuous ECG telemetry monitor, a stress testing system, an extended monitoring device, a smartphone-enabled ECG medical device, an external cardioverter-defibrillator therapy device, a subcutaneous implantable cardioverter defibrillators (S-ICD), an intracardiac or transvenous pacemaker device, an automated external defibrillators (AED), or an automatic implantable cardioverter defibrillator (AICD). In another aspect, the computing device is integrated into the one or more sensors or devices; or the one or more sensors or devices are integrated into the computing device. In another aspect, determining the changes in ventricular repolarization between the wide complex heart beat T-wave amplitudes and/or time-voltage areas and the baseline heart beat T-wave amplitudes and/or time-voltage areas comprises: receiving a wide complex heart beat waveform duration via the input/output interface or the memory; determining, using the one or more processors, a percent amplitude change (PAC) based on the wide complex heart beat T-wave amplitudes and the baseline T-wave amplitudes, and/or a percent time-voltage area change (PTVAC) based on the wide complex heart beat T-wave time-voltage areas and the baseline T-wave time-voltage areas; determining a classification probability based on the wide complex heart beat waveform duration, and the T-wave PAC and/or the T-wave PTVAC using the one or more processors; and wherein the signal change comprises the classification probability, and the classification probability comprises a VT probability, a SWCT probability, or a ventricular pacing probability. In another aspect, determining the classification probability is further determined based one or more additional classification predictors. In another aspect, the one or more classification predictors comprise changes in ventricular repolarization. In another aspect, the T-wave PAC comprises a frontal T-wave PAC and a horizontal T-wave PAC, and the T-wave PTVAC comprises a frontal T-wave PTVAC and a horizontal T-wave PTVAC.

In another aspect, determining the changes in ventricular repolarization between the wide complex heart beat T-wave amplitudes and/or time-voltage areas and the baseline heart beat T-wave amplitudes and/or time-voltage areas comprises: receiving a WCT QRS duration via the input/output interface or the memory; the one or more wide complex heart beat T-wave amplitudes and/or time-voltage areas comprise one or more frontal plane WCT positive T-wave amplitudes and/or time-voltage areas, one or more horizontal plane WCT positive T-wave amplitudes and/or time-voltage areas, one or more frontal plane WCT negative T-wave amplitudes and/or time-voltage areas, and one or more horizontal plane WCT negative T-wave amplitudes and/or time-voltage areas; the one or more the baseline heart beat T-wave amplitudes and/or time-voltage areas comprise one or more frontal plane baseline positive T-wave amplitudes and/or time-voltage areas, one or more horizontal plane baseline positive T-wave amplitudes and/or time-voltage areas, one or more frontal plane baseline negative T-wave amplitudes and/or time-voltage areas, and one or more horizontal baseline negative T-wave amplitudes and/or time-voltage areas; determining (1) a frontal T-wave percent amplitude change (PAC) based on the one or more frontal plane WCT positive T-wave amplitudes, one or more frontal plane WCT negative T-wave amplitudes, one or more frontal plane baseline positive T-wave amplitudes, and one or more frontal plane baseline negative T-wave amplitudes, and/or (2) a frontal T-wave percent time-voltage area (PTVAC) based on the one or more frontal plane WCT positive T-wave time-voltage areas, one or more frontal plane WCT negative T-wave time-voltage areas, one or more frontal plane baseline positive T-wave time-voltage areas, and one or more frontal plane baseline negative T-wave time-voltage areas; determining (1) a horizontal T-wave PAC based on the one or more horizontal plane WCT positive T-wave amplitudes, one or more horizontal plane WCT negative T-wave amplitudes, one or more horizontal plane baseline positive T-wave amplitudes, and one or more horizontal baseline negative T-wave amplitudes, and/or (2) a horizontal T-wave PTVAC based on the one or more horizontal plane WCT positive T-wave time-voltage areas, one or more horizontal plane WCT negative T-wave time-voltage areas, one or more horizontal plane baseline positive T-wave time-voltage areas, and one or more horizontal baseline negative T-wave time-voltage areas; determining a VT probability using a statistical or machine learning process based on the WCT duration and (1) the frontal T-wave PAC and the horizontal T-wave PAC, and/or (2) the frontal T-wave PTVAC and the horizontal T-wave PTVAC; and wherein the changes in ventricular repolarization comprises the VT probability. In another aspect, the statistical or machine learning process comprises a linear regression algorithm, a logistic regression model, a linear discriminate analysis algorithm, a Naive Bayes algorithm, a computational model using artificial neural networks, a computational model based on classification or regression trees, a k-nearest neighbors based model, a support vector machine based model, a boosting algorithm, or an ensemble machine learning algorithm.

In another aspect, the input/output interface comprises a remote device, and the remote device is communicably coupled to the one or more processors via one or more networks. In another aspect, the method further comprises providing a recommendation to select or exclude a therapy, medication, diagnostic testing or referral for a patient based on the signal change. In another aspect, the computing device comprises a server computer, a workstation computer, a laptop computer, a mobile communications device, a personal data assistant, or a medical device. Moreover, the method can be implemented using a non-transitory computer readable medium that when executed causes the one or more processors to perform the method.

Another embodiment of the present invention provides an apparatus for classifying a wide complex heart beat(s) comprising an input/output interface, a memory, and one or more processors communicably coupled to the input/output interface and the memory. The one or more processors: receive one or more wide complex heart beat waveform amplitudes and/or time-voltage areas of ventricular repolarization, and one or more baseline heart beat waveform amplitudes and/or time-voltage areas of ventricular repolarization via the input/output interface or the memory, determine a signal change in ventricular repolarization between the wide complex heart beat waveform amplitudes and/or time-voltage areas of ventricular repolarization and the baseline heart beat waveform amplitudes and/or time-voltage areas of ventricular repolarization using the one or more processors, and provide the signal change in ventricular repolarization via the input/output interface, wherein the signal change in ventricular repolarization provides an indication whether the wide complex heart beat(s) is from a ventricular source or a supraventricular source.

In one aspect, the signal change in ventricular repolarization further provides the indication whether the wide complex heart beat(s) is due to ventricular pacing or a premature ventricular contraction (PVC). In another aspect, the wide complex heart beat(s) comprise a wide complex tachycardia (WCT), the ventricular source comprises a ventricular tachycardia (VT), and the supraventricular source comprises a supraventricular wide complex tachycardia (SWCT). In another aspect, the signal change in ventricular repolarization comprises a VT probability, the wide complex heart beat classification comprises a VT whenever the VT probability is greater than or equal to the predetermined value, and the wide complex heart beat classification comprises a SWCT whenever the VT probability is less than the predetermined value. In another aspect, the one or more processors select the predetermined value from a range of 0% to 100%. In another aspect, the predetermined value comprises about 1%, 10%, 25%, 50%, 75%, 90% or 99%. In another aspect, the one or more processors provide the signal change in ventricular repolarization by providing a “shock” recommendation signal, a “no shock” recommendation signal, or no signal. In another aspect, the wide complex heart beat waveform amplitudes and/or time-voltage areas of ventricular repolarization comprise T wave amplitudes and/or time-voltage areas, the baseline heart beat waveform amplitudes and/or time-voltage areas of ventricular repolarization comprise baseline T wave amplitudes and/or time-voltage ares, and the T wave amplitudes and/or time-voltage areas and baseline T wave amplitudes and/or time-voltage areas are obtained from an electrocardiogram (ECG) signal, a ventricular electrogram (EMG) signal, a vectorcardiogram (VCG) and/or a mathematically-synthesized VCG signal. In another aspect, the wide complex heart beat T wave amplitudes and/or time-voltage areas comprise a plurality of measured T wave amplitudes and/or time-voltage areas of a ECG waveform, a EMG waveform, a VCG waveform, and/or a mathematically-synthesized VCG waveform above and below an isoelectric baseline; and the baseline heart beat T wave amplitudes and/or time-voltage areas comprise a plurality of measured T wave amplitudes and/or time-voltage areas of a baseline ECG waveform, a baseline EMG waveform, a baseline VCG waveform, and/or a baseline mathematically-synthesized VCG waveform above and below the isoelectric baseline.

In another aspect, the one or more processors receive the one or more wide complex heart beat waveform amplitudes and/or time-voltage areas of ventricular repolarization, and one or more baseline heart beat waveform amplitudes and/or time-voltage areas of ventricular repolarization by: receiving a ECG data, a EMG data, a VCG data, and/or a mathematically-synthesized VCG data via the input/output interface or the memory; receiving a baseline ECG data, a baseline EMG data, baseline VCG data, and/or baseline mathematically-synthesized VCG data via the input/output interface or the memory; determining the one or more waveform amplitudes and/or time-voltage areas of ventricular repolarization from the ECG data, the EMG data, the VCG data, and/or the mathematically-synthesized VCG data; and determining the one or more baseline waveform amplitudes and/or time-voltage areas of ventricular repolarization from the baseline ECG data, the baseline EMG data, the baseline VCG data, and/or the baseline mathematically-synthesized VCG data. In another aspect, the ECG data, the EMG data, theVCG data, and/or the mathematically-synthesized VCG data is generated or recorded before or after the baseline ECG data, the baseline EMG data, the baseline VCG data, and/or the baseline mathematically-synthesized VCG data. In another aspect, the ECG data, the EMG data, the VCG data and/or the mathematically-synthesized VCG data is generated or recorded after the baseline ECG data, the baseline EMG data, the baseline VCG data, and/or the baseline mathematically-synthesized VCG data and determining the signal change in ventricular repolarization. In another aspect, the ECG data, the EMG data, the VCG data, and/or the mathematically-synthesized VCG data and the baseline ECG data, the baseline EMG data, the baseline VCG data, and/or the baseline mathematically-synthesized VCG data are generated or recorded using one or more sensors or devices. In another aspect, the one or more sensors or devices comprise a 12-lead ECG device, a continuous ECG telemetry monitor, a stress testing system, an extended monitoring device, a smartphone-enabled ECG medical device, a cardioverter-defibrillator therapy device, a subcutaneous implantable cardioverter defibrillator (S-ICD), an intracardiac or transvenous pacemaker device, an automated external defibrillator (AED), or an automatic implantable cardioverter defibrillator (AICD). In another aspect, the input/output interface, the memory and the one or more processors are integrated into the one or more sensors or devices; or the one or more sensors or devices are integrated into a computing device comprising the input/output interface, the memory and the one or more processors. In another aspect, the one or more processors determine the signal change in ventricular repolarization between the wide complex heart beat waveform amplitudes and/or time-voltage areas of ventricular repolarization and the baseline heart beat waveform amplitudes and/or time-voltage areas of ventricular repolarization by: receiving a wide complex heart beat QRS waveform duration via the input/output interface or the memory; determining a percent T-wave amplitude change (PAC) based on the wide complex heart beat waveform amplitudes of ventricular repolarization and the baseline heart beat waveform amplitudes of ventricular repolarization, and/or a percent T-wave time-voltage area change (PTVAC) based on the wide complex heart beat waveform time-voltage areas of ventricular repolarization and the baseline heart beat waveform time-voltage areas of ventricular repolarization; determining a classification probability based on the wide complex heart beat waveform QRS duration, and the T-wave PAC and/or the T-wave PTVAC; and wherein the signal change in ventricular repolarization comprises the classification probability, and the classification probability comprises a VT probability, a SWCT probability, a premature ventricular contraction (PVC) probability, a singular supraventricular contraction (e.g. premature atrial contraction) probability or a ventricular pacing probability. In another aspect, determining the classification probability is further determined based one or more additional classification predictors. In another aspect, the PAC comprises a frontal T-wave PAC and a horizontal T-wave PAC, and the T-wave PTVAC comprises a frontal T-wave PTVAC and a horizontal T-wave PTVAC.

In another aspect, the one or more processors determine the signal change in ventricular repolarization between the wide complex heart beat waveform amplitudes and/or time-voltage areas of ventricular repolarization and the baseline heart beat waveform amplitudes and/or time-voltage areas of ventricular repolarization by: receiving a WCT QRS duration via the input/output interface or the memory; the one or more wide complex heart beat waveform amplitudes and/or time-voltage areas of ventricular repolarization comprise one or more frontal plane WCT positive T wave amplitudes and/or time-voltage areas, one or more horizontal plane WCT positive T wave amplitudes and/or time-voltage areas, one or more frontal plane WCT negative T wave amplitudes and/or time-voltage areas, and one or more horizontal plane WCT negative T wave amplitudes and/or time-voltage areas; the one or more the baseline heart beat waveform amplitudes and/or time-voltage areas of ventricular repolarization comprise one or more frontal plane baseline positive T wave amplitudes and/or time-voltage areas, one or more horizontal plane baseline positive T wave amplitudes and/or time-voltage areas, one or more frontal plane baseline negative T wave amplitudes and/or time-voltage areas, and one or more horizontal baseline negative T wave amplitudes and/or time-voltage areas; determining (1) a frontal T-wave percent amplitude change (PAC) based on the one or more frontal plane WCT positive T wave amplitudes, one or more frontal plane WCT negative T wave amplitudes, one or more frontal plane baseline positive T wave amplitudes, and one or more frontal plane baseline negative T wave amplitudes, and/or (2) a frontal T-wave percent time-voltage area (PTVAC) based on the one or more frontal plane WCT positive T wave time-voltage areas, one or more frontal plane WCT negative T wave time-voltage areas, one or more frontal plane baseline positive T wave time-voltage areas, and one or more frontal plane baseline negative T wave time-voltage areas; determining (1) a horizontal T-wave PAC based on the one or more horizontal plane WCT positive T wave amplitudes, one or more horizontal plane WCT negative T wave amplitudes, one or more horizontal plane baseline positive T wave amplitudes, and one or more horizontal baseline negative T wave amplitudes, and/or (2) a horizontal T-wave PTVAC based on the one or more horizontal plane WCT positive T wave time-voltage areas, one or more horizontal plane WCT negative T wave time-voltage areas, one or more horizontal plane baseline positive T wave time-voltage areas, and one or more horizontal baseline negative T wave time-voltage areas; determining a VT probability using a statistical or machine learning process based on the WCT duration and (1) the frontal T-wave PAC and the horizontal T-wave PAC, and/or (2) the frontal T-wave PTVAC and the horizontal T-wave PTVAC; and wherein the signal change in ventricular repolarization comprises the VT probability. In another aspect, the statistical or machine learning process comprises a linear regression algorithm, a logistic regression model, a linear discriminate analysis algorithm, a Naive Bayes algorithm, a computational model using artificial neural networks, a computational model based on classification or regression trees, a k-nearest neighbors based model, a support vector machine based model, a boosting algorithm, or an ensemble machine learning algorithm.

In another aspect, the input/output interface comprises a remote device, and the remote device is communicably coupled to the one or more processors via one or more networks. In another aspect, the one or more processors provide a recommendation to select or exclude a therapy, medication, diagnostic testing or referral for a patient based on the signal change in ventricular repolarization. In another aspect, apparatus comprises a server computer, a workstation computer, a laptop computer, a mobile communications device, a personal data assistant, or a medical device.

### BRIEF DESCRIPTION OF THE DRAWINGS

For a more complete understanding of the features and advantages of the present invention, reference is now made to the detailed description of the invention along with the accompanying figures and in which:

FIG. 1A depicts a schematic representation of a normal ECG;

FIG. 1B depicts an example of ECG data collected and recorded from a patient's 12-lead ECG;

FIG. 2 depicts a schematic representation of the resultant QRS amplitude changes that manifest between a patient's baseline and WCT ECG;

FIGS. 3A-3E depicts panels that summarize the expected range of mean electrical vector of ventricular depolarization changes in the frontal ECG plane after WCT event onset;

FIGS. 4A-4E depicts panels that summarize the expected range of mean electrical vector changes of ventricular depolarization changes in the horizontal ECG plane after WCT event onset;

FIGS. 5A-5B are graphic depictions of select ECG lead combinations utilized by the frontal (aVR, aVL, aVF) and horizontal (V1, inverse V4, V6) PAC formulas in accordance with one embodiment of the present invention;

FIGS. 6A-6C depict the structure of the frontal PAC formula, horizontal PAC formula and amplitude based WCT formula in accordance with one embodiment of the present invention;

FIG. 7 depicts a flow diagram representing the inputs and output of the amplitude based WCT Formula in accordance with one embodiment of the present invention;

FIG. 8 illustrates the inclusion criteria and reasons for exclusion during validation cohort selection for the WCT Formula validation;

FIG. 9 is Table 1 showing the ECG characteristics of the derivation cohort;

FIG. 10 is Table 2 showing the clinical characteristics of the derivation cohort;

FIG. 11 is Table 3 showing the mean and standard deviation (SD) of measured and calculated ECG variables among VT or SWCT groups within the derivation cohort;

FIGS. 12A-12C are box-plots demonstrating the median and proportional distribution of WCT QRS duration (ms) (FIG. 12A), frontal PAC (%) (FIG. 12B) and Horizontal PAC (%) (FIG. 12C) for VT and SWCT groups in accordance with one embodiment of the present invention;

FIG. 12D is a table showing electrocardiographic variables among baseline ECG sub-groups in accordance with on embodiment of the present invention;

FIG. 13 is a graph of a receiver operating characteristic (ROC) curve depicting amplitude based WCT Formula diagnostic performance in accordance with one embodiment of the present invention;

FIG. 14 is Table 4 showing the ECG characteristics of the validation cohort;

FIG. 15 is Table 5 showing the clinical characteristics of the validation cohort;

FIGS. 16A and 16B are histograms demonstrating the distribution of clinically diagnosed VT and SWCT according to the amplitude based WCT Formula diagnostic performance at on probability estimates (0.000%-99.999%) for the validation cohort;

FIG. 17 is Table 6 showing the diagnostic performance of various VT probability partitions for the validation cohort in accordance with one embodiment of the present invention;

FIGS. 18A and 18B are Venn diagrams summarizing the distribution of shared and non-shared VT (FIG. 18A) and SWCT (FIG. 18B) diagnoses established by three diagnostic standards for the validation cohort: (1) clinical diagnosis, (2) ECG laboratory interpretation and (3) amplitude based WCT Formula's 50% VT probability partition;

FIGS. 19A and 19B are tables showing the electrocardiographic characteristics of clinical SWCT classified as VT and clinical VT classified as SWCT by the amplitude based WCT Formula's 50% VT probability partition for the validation cohort;

FIG. 20 depicts a schematic representation of a normal ECG with time-voltage areas;

FIGS. 21A-21B are graphic depictions of select ECG lead combinations utilized by the frontal (aVR, aVL, aVF) and horizontal (V1, inverse V4, V6) PAC formulas with respect to time-voltage areas in accordance with one embodiment of the present invention;

FIG. 22 depicts a schematic representation of the resultant QRS time-voltage area changes that manifest between a patient's baseline and WCT ECG;

FIGS. 23A-23C depict derivations of the frontal PTVAC formula, horizontal PTVAC formula and time-voltage are based WCT formula in accordance with one embodiment of the present invention;

FIGS. 24A-24B are box-plots demonstrating the median and proportional distribution of frontal PTVAC (%) (FIG. 24A) and horizontal PTVAC (%) (FIG. 24B) for VT and SWCT groups in accordance with one embodiment of the present invention;

FIGS. 25A-25B are ROC graphs depicting the diagnostic performance of frontal PTVAC (%) (FIG. 25A) and horizontal PTVAC (%) (FIG. 25B) in accordance with one embodiment of the present invention;

FIG. 26 is a graph depicting the time-voltage area based WCT Formula's diagnostic performance for the derivation cohort (AUC of 0.95) in accordance with one embodiment of the present invention;

FIGS. 27A and 27B are histograms demonstrating the distribution of clinical VT and SWCT according to the time-voltage area based WCT Formula diagnostic performance at VT probability estimates (0.000%-99.999%) in accordance with one embodiment of the present invention;

FIG. 28 is a graph depicting the VCG-VT Model's diagnostic performance for the testing cohort (AUC of 0.97) in accordance with one embodiment of the present invention;

FIG. 29 is a block diagram of an apparatus in accordance with one embodiment of the present invention;

FIG. 30 is a flow chart of a method in accordance with one embodiment of the present invention;

FIG. 31 is a flow diagram of validation cohort selection for the VT Prediction Model validation;

FIGS. 32A-32B are examples of paired VT (FIG. 32A) and baseline (FIG. 32B) ECGs assigned high VT probability (99.0006%) by the VT Prediction Model in accordance with one embodiment of the present invention;

FIGS. 33A-33B are examples of paired SWCT (FIG. 33A) and baseline (FIG. 33B) ECGs assigned low VT probability (4.3609%) by the VT Prediction Model in accordance with one embodiment of the present invention;

FIGS. 34A-34B are examples of paired SWCT (FIG. 34A) and baseline (FIG. 34B) ECGs assigned low VT probability (6.3613%) by the VT Prediction Model in accordance with one embodiment of the present invention;

FIG. 35 is a table showing the clinical and ECG laboratory diagnosis;

FIG. 36 is a table showing the patient characteristics;

FIG. 37 is a table showing the electrocardiographic variables;

FIG. 38 is a table showing the electrocardiographic variables among baseline ECG sub-groups;

FIG. 39A-39D are box-plots demonstrating the median and proportional distribution of WCT QRS duration (ms) (FIG. 39A), WCT QRS duration change (ms)

(FIG. 39B), QRS axis change (°) (FIG. 39C) and T axis change (°) (FIG. 39D) in accordance with one embodiment of the present invention;

FIG. 40 is a graph depicting a receiver operating characteristic curve for the VT Prediction Model (AUC of 0.942) in accordance with one embodiment of the present invention;

FIG. 41 is a table showing the percent VT probability partitions in accordance with one embodiment of the present invention;

FIG. 42 is a table showing the correct and erroneous WCT diagnoses in accordance with one embodiment of the present invention;

FIG. 43 is a table summarizing the clinical diagnosis and ECG laboratory interpretation data of the validation cohort in accordance with one embodiment of the present invention;

FIG. 44 is a table summarizing the patient characteristics of VT and SWCT groups for the validation cohort in accordance with one embodiment of the present invention;

FIG. 45 is a graph depicting a receiver operating characteristic curve for the VT Prediction Model (AUC of 0.900; CI 0.862 to 0.939) in accordance with one embodiment of the present invention;

FIGS. 46A and 46B are histograms demonstrating the distribution of VT and SWCT according to the VT Prediction Model's VT probability estimates in accordance with one embodiment of the present invention;

FIGS. 47A-47E depicts panels that summarize the expected changes to the mean electrical vector of ventricular depolarization following WCT initiation;

FIGS. 48A-48E depicts panels that summarize the expected changes to the mean electrical vector of ventricular repolarization upon WCT initiation;

FIGS. 49-49B are examples of paired VT (FIG. 49A) and baseline (FIG. 49B) ECGs assigned low VT probability (9.8704%) by the VT Prediction Model in accordance with one embodiment of the present invention;

FIGS. 50A-50B are examples of paired SWCT (FIG. 50A) and baseline (FIG. 50B) ECGs assigned high VT probability (54.0039%) by the VT Prediction Model in accordance with one embodiment of the present invention;

FIG. 51 is a flow chart of a method in accordance with another embodiment of the present invention; and

FIG. 52 is a flow chart of a method in accordance with another embodiment of the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Three types of embodiments of the present invention are described herein. Each type of embodiment may include numerous variations as described below. The first type is based in whole or in part on a wide complex tachycardia (WCT) Formula. The second type is based in whole or in part on a ventricular tachycardia (VT) prediction model. The third type is based in whole or in part on an analysis of ventricular repolarization (e.g., T wave). Note that the variables in each of these three embodiments can be combined with variables from the other embodiments (e.g., a prediction model that utilizes both repolarization changes and depolarization changes, etc.).

WCT Formula Embodiments

One type of embodiments of the present invention provides a new electrophysiological principle (degree of QRS or ventricular electrogram signal change in amplitude and/or time-voltage area between the WCT and baseline heart rhythm helps distinguish ventricular tachycardia (VT) and supraventricular wide complex tachycardia (SWCT)) that can be exploited by ECG, EMG, VCG, and mathematically-synthesized VCG interpretation software to render precise and accurate predictions of VT verses SWCT. The WCT differentiation method described herein can be automatically implemented by contemporary ECG interpretation software. Note that other medical devices that analyze ECG signals, electrogram (EMG) signals, vectorcardiogram (VCG) signals and/or mathematically-synthesized VCG signals from the heart (e.g pacemakers, transvenous lead or subcutaneous automated implantable cardioverter-defibrillators, automated external defibrillators) can utilize the similar methods or systems based on the foregoing principles of this present invention. Note that other formulas or algorithms based on the foregoing principle and other information described herein can be used to predict the source of a wide complex beat (supraventricular or ventricular) by diagnostic interpretation software analysing ECG, EMG, VCG, and mathematically-synthesized VCG signals. As a result, the present invention is not limited to the WCT Formulas described herein.

The WCT Formulas were designed to effectively, accurately and automatically differentiate WCT into VT, which is usually a dangerous heart rhythm, and SWCT, which is ordinarily a less hazardous heart rhythm. VT and SWCT are most often non-invasively diagnosed using a 12-lead ECG. However, the present invention is applicable to any current or future technology that provides the relevant data using known or unknown detection devices or sensors (i.e., any device that generates and analyzes ECG signals, ventricular EMG signals, VCG signals and/or mathematically synthesized VCG signals).

The WCT Formulas are logistic regression models that deliver an automatic prediction VT likelihood (i.e., % VT probability) using ECG measurements (e.g. WCT duration) and calculations (e.g., frontal and horizontal Percent Amplitude Change (PAC), or frontal and horizontal Percent Time-Voltage Area Change (PTVAC)) derived from paired WCT and baseline ECGs. The frontal and horizontal PAC and PTVAC formulas are highly predictive determinants of VT and SWCT, wherein a low PAC (%) or PTVAC (%) indicates SWCT and a high PAC (%) or PTVAC (%) indicates VT. Moreover, the frontal and horizontal PAC or PTVAC calculations are independent predictors of VT. Each calculation is able to provide a reliable means to effectively distinguish VT and SWCT. They can also be used to differentiate discrete ventricular depolarizations due to premature ventricular contractions, ventricular pacing, and wide complex beats from a supraventricular source.

The WCT Formula using amplitudes will be described in detail below. The WCT Formula using time-voltage areas will be described thereafter.

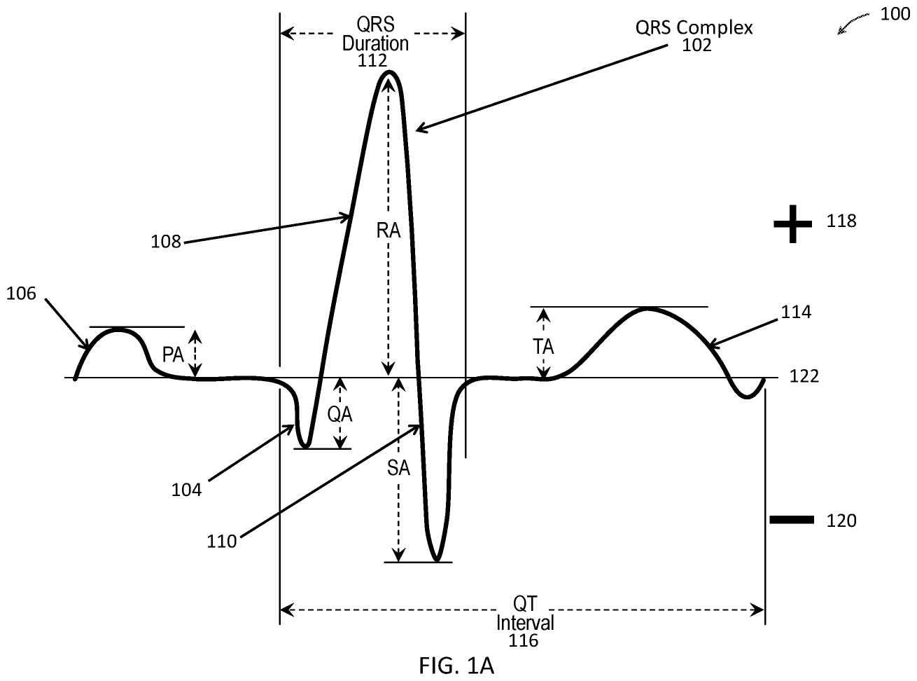

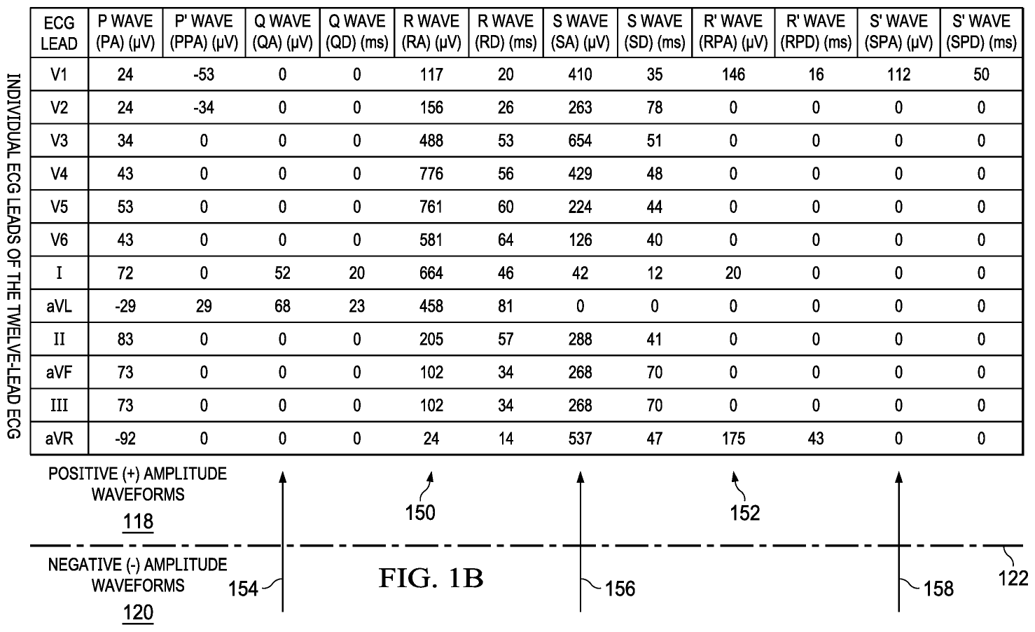

Now referring to FIGS. 1A and 1B, the 12-lead ECG and resulting data used in the WCT Formula will be described. The ECG currently is the most commonly used test to determine whether a patient's underlying heart rhythm is normal or abnormal. The 12-lead ECG records the electrical activity of the heart using 12 separate leads. Each lead records unique QRS complexes representative of the heart's ventricular depolarization. FIG. 1A is a schematic representation of a stereotypical ECG pattern for a single heart beat 100. The QRS complex waveform 102 is the combination of three graphical deflections: (1) the Q wave 104 having a downward deflection immediately following the P wave 106; (2) the R wave 108 having an upward deflection immediately following the Q wave 104; and (3) the S wave 110 having a downward deflection following the R wave 108. The Q wave 104, R wave 108 and S wave 110 occur in rapid succession and are encompassed within the QRS complex waveform 102 and accompanying time interval, QRS duration 112. The T-wave 114 follows the S wave 110. Each wave has amplitude denoted as PA, QA, RA, SA and TA. In addition, the QT interval 116 is the time interval extending from the onset of the QRS complex waveform 102 to the end of the T wave 114. The QRS complex 102 is divided into positive (+) amplitudes 118 and negative (−) amplitudes 120. The positive (+) amplitudes 118 are the vertical QRS complex deflections above the isoelectric baseline 122, namely the amplitude of r/R wave and r′/R′ wave. The negative (−) amplitudes 120 are the vertical QRS complex deflections below the isoelectric baseline 122, namely the amplitude of q or QS wave, s/S wave and s′/S′ wave. In addition computerized ECG interpretation software, such as the MUSE provided by GE Healthcare, automatically measures QRS complex waveform 102 attributes, namely q or QS, r/R, s/S, r′/R′, s′/S′ durations (ms), amplitudes (μV), and time-voltage areas (μV·ms) Note that standard annotation of QRS complex waveforms of small QRS waveforms are in lower case and large QRS waveforms are in upper case.

FIG. 1B depicts a measurement matrix showing an example of 12-lead ECG data recorded and calculated by computerized ECG interpretation software. The 12 leads are denoted as V1, V2, V3, V4, V5, V6, I, aVL, II, aVF, III, and aVR. In this example, QRS waveform deflection (q or QS, r/R, s/S, r′/R′, s′/S′) measurements including duration (ms) and amplitude (μV) are provided by GE Healthcare's MUSE ECG interpretation software and databank. In this example, the amplitude (_A) and duration (_D) data for the various waves are denoted as PA, PPA, QA, QD, RA, RD, SA, SD, RPA, RPD, SPA, and SPD. Note that other computerized ECG interpretation software can be used to derive this electrocardiographic data. Note that these measurements are not routinely shown on the ECG paper recording, but are available within the ECG interpretation software databanks. The positive (+) amplitudes 118 are the vertical QRS complex deflections above the isoelectric baseline 122, namely the r/R wave amplitude (μV) 150 and r′/R′ wave amplitude (μV) 152. The negative (−) amplitudes 120 are the vertical QRS complex deflections below the isoelectric baseline 122, namely the q or QS wave amplitude (μV) 154, s/S wave amplitude (μV) 156, and s′/S′ wave amplitude (μV) 158. As will be described in more detail below, the voltage amplitude measurements from specific leads (frontal ECG plane: V1, V4, V6; and horizontal ECG plane: aVL, aVF, aVR) are used in the frontal and horizontal PAC formulas to generate the frontal and horizontal PACs (%).

Note that contemporary computerized ECG interpretation software also routinely provides standard ECG measurements including QRS duration (ms), QTc duration (ms), and frontal plane R and T wave axes (°). These measurements are typically apparent/reported on the 12-lead ECG paper recording. The difference in QRS duration (ms), frontal plane R wave axis (°) and frontal plane T wave axis (°) between the WCT and baseline ECGs may be automatically calculated by computerized ECG interpretation software. Note that time-voltage area measurements of separate QRS waveform deflections (q or QS, r/R, s/S, r′/R′, s′/S′) deflections can be automatically provided by computerized ECG interpretation software and electronic databanks (e.g., MUSE from GE Healthcare, etc.).

The new electrophysiology principles that are the backbone of the horizontal and frontal PAC formulas will now be described. The number of ways VT may propagate within and depolarize the ventricular myocardium is ostensibly limitless. Consequently, VTs have an immeasurable number of ways they can be electrocardiographically distinct from their respective baseline ECG. In contrast, the manner SWCTs depolarize the ventricular myocardium is ordinarily confined to the same His-Purkinje network or implantable device system pathways utilized by the baseline heart rhythm; in rarer instances SWCTs may be due to ventricular pre-excitation using separate atrioventricular accessory pathways. As a result, many SWCTs, especially those with pre-existing aberrancy or ventricular pacing, demonstrate substantial electrocardiographic similarity with the baseline ECG. On the contrary, SWCTs with “functional” aberration exhibit recognizably different QRS complex configurations. However, since most functional SWCTs demonstrate antegrade impulse propagation and ventricular depolarization confined in the His-Purkinje network, they are destined to express a relatively constrained variety of electrocardiographically distinct QRS complexes.

Moreover, the amplitude and time-voltage area based WCT Formulas (and its principles) can be similarly applied to these types of defibrillator devices because they either use ECG signals using surface ECG electrodes (or a modification thereof with the subcutaneous ICD) or EMG signals derived from intracardiac and extracardiac electrodes (in the case of AICDs and pacemakers) to help distinguish different heart rhythms. Because these devices acquire ventricular depolarization signals from surface ECG electrodes or EMG electrodes, the invention, and its principles of QRS (or ventricular EMG signal) amplitude (or time-voltage area) change, can be applied to help them more accurately discriminate SWCT and VT.

Provided the means by which VT may propagate within and depolarize the ventricular myocardium is essentially unlimited, VTs have an expansive means to which their ventricular electrograms (EMGs) may be morphologically distinct from the ventricular EMGs of the baseline heart rhythm. In contrast, the manner SWCTs depolarize the ventricular myocardium is ordinarily confined to the same His-Purkinje network or implantable device system utilized by the baseline heart rhythm; in rarer instances SWCTs may be to ventricular pre-excitation using separate atrioventricular accessory pathways. As a result, many ventricular EMGs from SWCTs, especially those with pre-existing aberrancy or ventricular pacing, demonstrate marked similarity with the ventricular EMGs for the patient's baseline heart rhythm. On the contrary, SWCTs with “functional” aberration exhibit recognizably different ventricular EMG configurations. However, since functional SWCTs still demonstrate antegrade impulse propagation and ventricular depolarization confined in the His-Purkinje network, they tend to express a relatively constrained variety of ventricular EMG complexes.

As a result, various embodiments of the present invention can be used to further help guide therapy decisions (e.g., “shock patient” for VT OR “do not shock the patient” for SWCT). As a consequence, the likelihood of appropriate device defibrillations (i.e., appropriate shocks) may be increased while decreasing the likelihood of inappropriate device defibrillations (i.e. inappropriate shocks).

Likewise, various embodiments of the present invention can be used by conventional transvenous lead based devices like AICDs or pacemakers or new intracardiac devices (e.g. Micra Transcatheter Pacing System). These devices analyze multiple separate bipolar EMG signals derived from various intracardiac and extracardiac electrodes combinations (e.g., right ventricular coil to AICD generator housing OR extracardiac SVC coils to AICD generator housing OR RV right ventricular tip to right ventricular coil OR any other combination). In general, commercially available implanted devices usually store 2-4 EMG channels which are analyzed by embedded interpretation algorithms. These EMG channels (separately or in combination) can be examined to establish the degree (or percentage) of ventricular EMG amplitude or time-voltage area change between the WCT and baseline EMG. This procedure/method can help distinguish VT and SWCT.

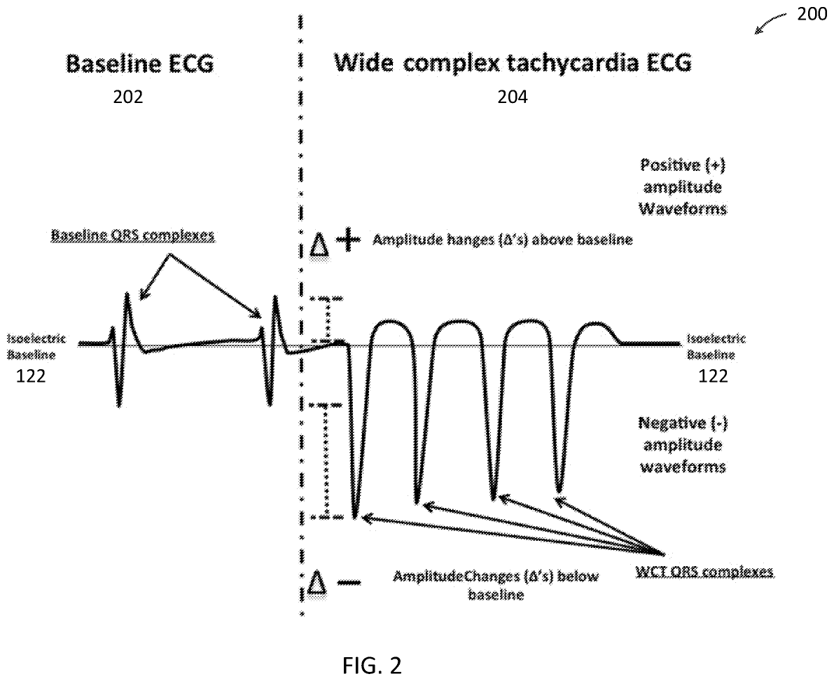

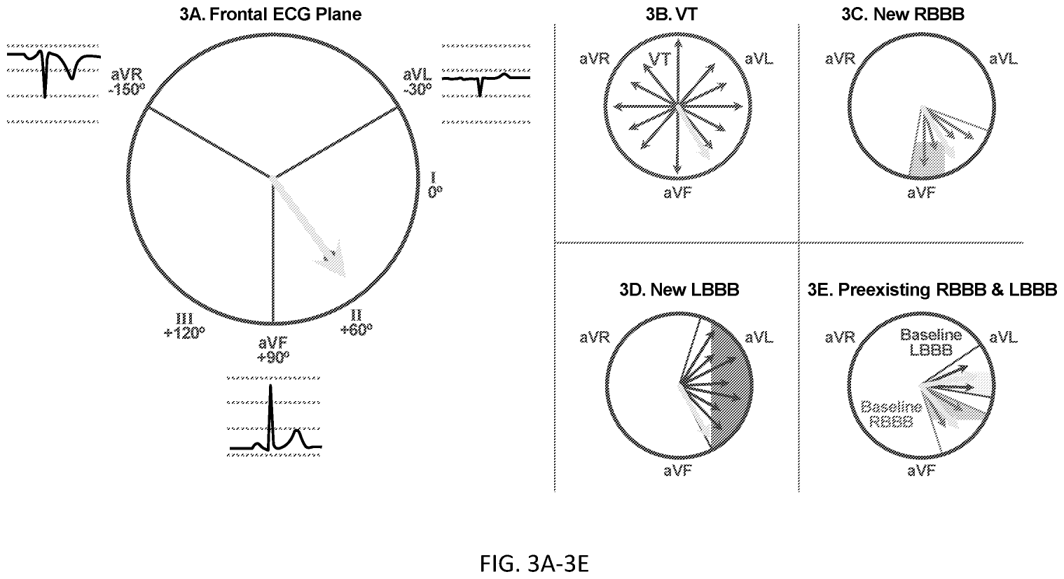

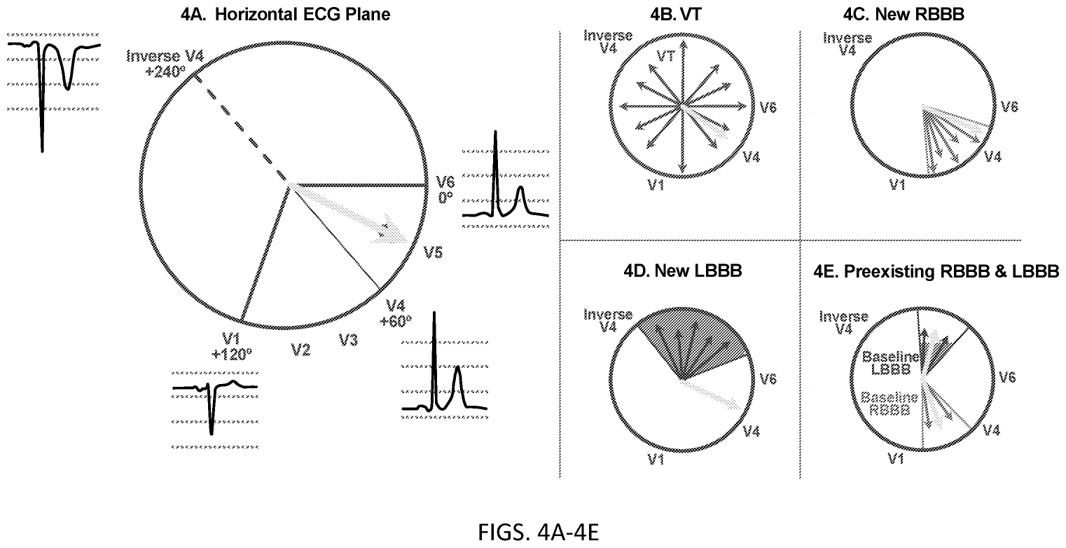

Referring now to FIG. 2, a schematic representation 200 of the resultant QRS amplitude changes that manifest between a patient's baseline ECG 202 and WCT ECG 204 is shown. The transition between a patient's baseline and WCT ECG (or vice versa) is inherently associated with changes (large or small) in QRS amplitude. Note the separate QRS amplitude changes (A's) that occur (+) above and (−) below the isoelectric baseline 122. Any change in QRS amplitude essentially signals attendant changes in the mean electrical vector of ventricular depolarization. Given VT's more expansive means of ventricular depolarization, it was hypothesized that it would ordinarily demonstrate greater changes to the mean electrical vector than SWCT in both the frontal and horizontal ECG planes (FIGS. 3A-3E and 4A-4E, respectively). To test this hypothesis, the frontal and horizontal PAC formulas were created to broadly delineate the extent of QRS amplitude change that manifests between the WCT and baseline ECG.

Both calculations determine the percent (%) change in QRS amplitude that occurs at specific ECG lead combinations within the frontal (aVR, aVL, aVF) or horizontal (V1, V4, V6) ECG plane. In order to detect and quantify changes in the net direction (i.e. axis) and/or voltage intensity of the mean electrical vector, each PAC calculation utilizes ECG leads that are in effect separated by approximately 120°. In the case of V4, its mathematical inverse equivalent, “inverse V4,” is separated equidistant from V1 and V6 by approximately 120°.

Now referring to FIGS. 3A-3E and 4A-4E, panels that summarize mean electrical vector changes in the frontal (FIG. 3A-3E) and horizontal (FIGS. 4A-4E) ECG planes after WCT event onset are shown. The mean electrical vector (of the frontal or horizontal ECG plane) represents the summative electrical vector of ventricular depolarization. This value is determined from the QRS amplitudes derived from the 12-lead ECG. Heavy arrows represent the mean electrical vector for an ECG with demonstrating normal sinus rhythm (Panels 3A-3D, 4A-4D) or pre-existing BBB (Panels 3E, 4E). Shaded regions depict the range of potential axes and voltage intensities for mean electrical vectors that occur after WCT onset. Select ECG leads utilized by the frontal (aVR, aVL, aVF) and horizontal (V1, V4, V6) PAC formulas are highlighted. Inverse V4 is the inverted equivalent of its planar opposite: lead V4. Panels 3A, 4A demonstrates the mean electrical vector for a typical normal sinus baseline ECG. Panels 3B-3E, 4B-4E demonstrate the expected range of mean electrical vectors following the onset of various WCTs. Panels 3B, 4B demonstrates VT's incredibly expansive range of potential mean electrical vectors. Panels 3C-3D, 4C-4D demonstrate the relatively constrained mean electrical vector changes for SWCTs due to functional RBBB (Panels 3C, 4C) and LBBB (Panels 3D, 4D). Panels 3E, 4E depict the minimal mean electrical vector changes for SWCTs with pre-existing aberrancy. As shown, SWCTs have “restricted” changes to the mean electrical vector that translates into smaller frontal and horizontal PACs, and VTs tend to demonstrate “expansive” changes in the mean electrical vector that translates into larger frontal and horizontal PACs. Therefore, VT demonstrates much greater frontal and horizontal PACs than SWCT. Correspondingly, the larger frontal and horizontal PACs strongly predict VT, whereas smaller frontal and horizontal PACs predicted SWCT.

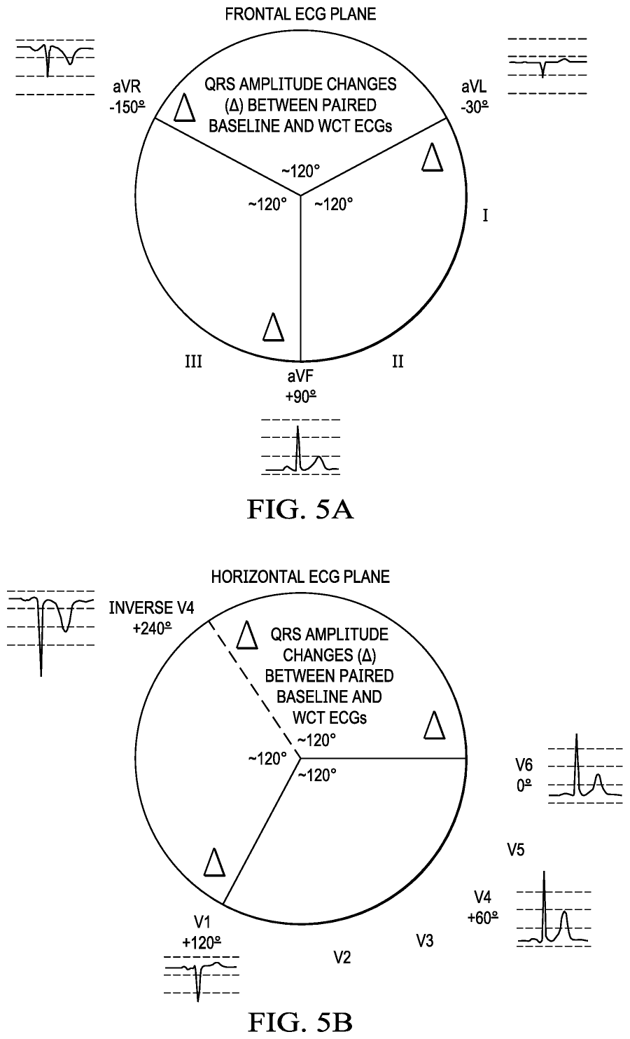

Referring now to FIGS. 5A-5B, graphic depictions of select ECG lead combinations utilized by the frontal (aVR, aVL, aVF)(FIG. 5A) and horizontal (V1, inverse V4, V6)(FIG. 5B) PAC formulas in accordance with one embodiment of the present invention are shown. The QRS amplitude change (Δ) that manifests between the baseline and WCT ECGs at these selected leads is the foundation for each PAC calculation. Note that the absolute QRS amplitude changes (Δ's) that manifest in lead V4 are mathematically equivalent to its planar opposite: inverse V4.

This present invention is in agreement with the multivariate logistic regression analysis reported by Griffith et al in 1991 (7). In their study, WCTs demonstrating large frontal plane QRS axis shifts (>=40°) from the baseline sinus rhythm ECG strongly predicted VT. Notably, they found QRS axis shifts to be the 3^{rd} strongest independent WCT predictor (after MI history and lead aVF QRS configuration) among 15 clinical and 11 electrocardiographic variables. Similar to the recognition of large frontal plane QRS axis shifts, each PAC calculation is able to detect sizable changes in the net direction (i.e. axis) of the mean electrical vector. Yet, more advantageously, both PAC calculations provide a workable means to quantify changes in the net direction, voltage intensity, and/or QRS morphologic configuration produced by ventricular depolarization.

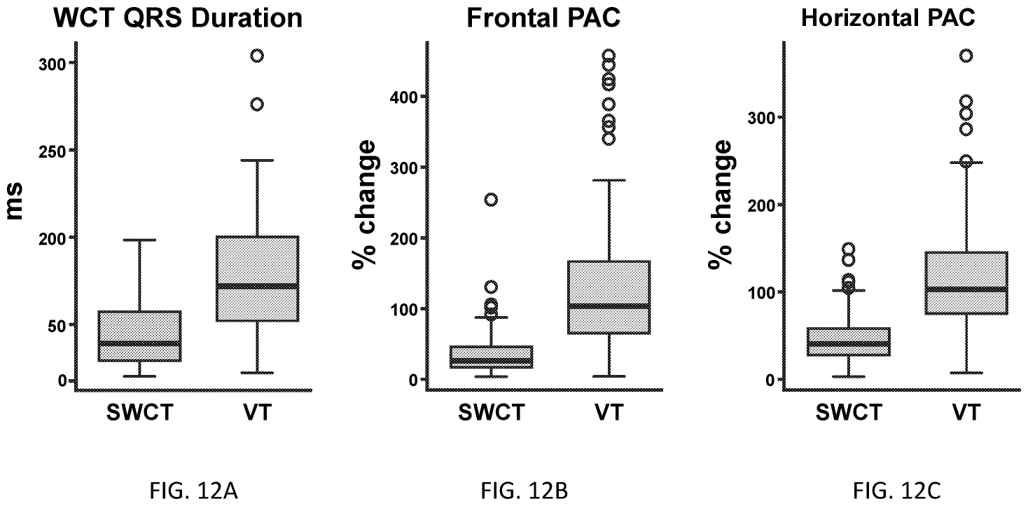

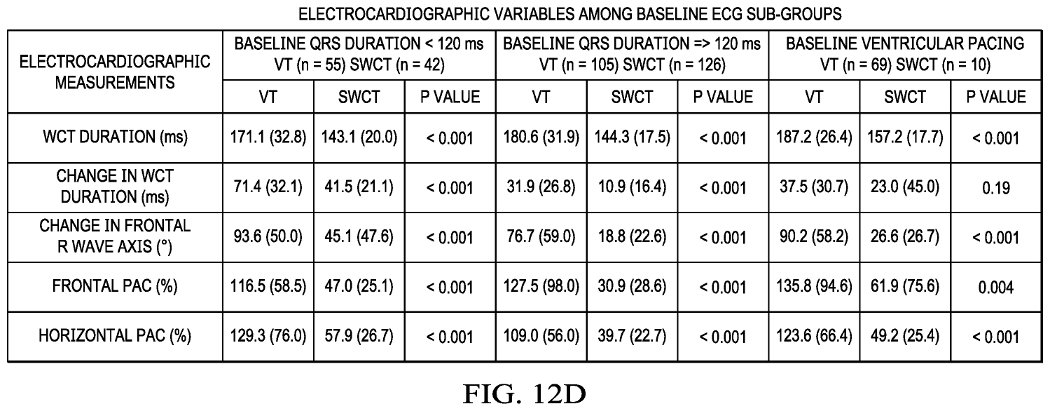

This present invention also agrees with the findings reported by Dongas et al in 1985 (16). Their study confirmed that WCTs with similar morphologic configurations as the pre-existing bundle branch block (BBB) were likely SWCTs, whereas WCTs with different morphologic configurations were likely VTs. Correspondingly, it was observed that SWCT demonstrated much smaller frontal and horizontal PACs than VT among ECG pairs with baseline QRS prolongation (QRS duration=>120 ms). However, it was furthermore observed that SWCT demonstrates smaller frontal and horizontal PACs than VT among ECG pairs without baseline QRS prolongation (QRS duration<120 ms) (see FIG. 12D).

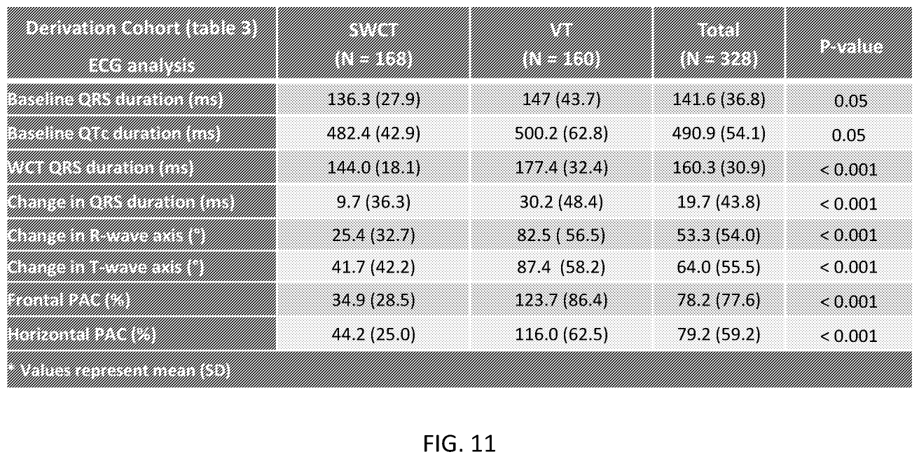

It is well known that WCTs with more prolonged QRS durations are less likely due to SWCTs with aberrant conduction. This observation was first described in 1978 by Wellens et al who showed that VTs generally demonstrate longer QRS durations than SWCTs with functional aberrancy (3). This understanding later evolved into proposed QRS duration cut-offs for VT diagnoses: QRS>140 ms for WCTs with right BBB configuration and QRS>160 ms for WCTs with left BBB configuration (4). However, subsequent study (17-20) has determined the sole use of QRS duration cut-offs to be problematic because SWCTs often demonstrate QRS durations greater than 160 ms. This most commonly occurs among patients with ongoing antiarrhythmic drug use, pre-existing BBB or advanced cardiomyopathy. In addition, several series have also shown that VTs often demonstrate QRS durations less than 140 ms (3, 4, 18, 19). This tends to occur among VTs that rapidly utilize the His-Purkinje network or develop in patients without structural heart disease. The findings described herein support that VTs demonstrate longer QRS durations than SWCTs (see e.g., FIG. 11).

A logistic regression formula (i.e. WCT Formula) capable of establishing accurate VT probability predictions using measurements and calculations provided by contemporary ECG interpretation software was created. Note that the present invention is not limited to use of a logistic regression model, such as the amplitude based WCT Formula. Other “machine learning” or artificial intelligence prediction methods (e.g., artificial neural networks, support vector machines, Random Forests, etc.) can be used with the frontal and horizontal PAC calculations. The amplitude based WCT Formula incorporates the strong independent WCT predictors including (1) WCT QRS duration (ms), (2) frontal PAC (%) and (3) horizontal PAC (%). The predictive contribution of each WCT predictor is concomitantly “weighed” according to their influence on the binary outcome (VT vs. SWCT) to render an exact VT probability estimation. Given each WCT predictor's direct relationship with VT likelihood, the amplitude based WCT Formula estimates higher VT probabilities for ECG pairs demonstrating greater WCT QRS durations, frontal PAC and/or horizontal PAC. Similarly, the amplitude based WCT Formula estimates lower VT probability for ECG pairs with smaller WCT QRS durations, frontal PAC and/or horizontal PAC.

The use of a multivariate logistic regression model to formulate the WCT Formulas allows (1) delivery of precise VT probability predictions and (2) later inclusion of other well-established, enhanced and/or newly formulated WCT predictors. The use of a step-wise decision-tree approach were avoided because of their tendency to prematurely commit to WCT diagnoses without sufficiently considering the predictive strengths of other relevant VT or SWCT predictors. The use of specific value cut-offs for VT diagnoses (e.g., QRS duration=>160 ms or frontal PAC>=75%) was avoided because this tends to cause (1) misclassifications due to VT and SWCT overlap and (2) ambiguity concerning the strength of WCT diagnoses for values distributed well above, well below, or at the margin of the designated cut-offs.

The WCT Formula's logistic regression model structure uses select independent WCT predictors (WCT QRS duration (ms), frontal PAC (%) and horizontal PAC (%)) to render a precise prediction of VT probability (%). Each WCT predictor (Xₓ) was assigned beta coefficients (βₓ) according to their influence on the binary outcome (VT vs. non-VT). The “constant” term (B₀) represents the y-intercept of the least squares regression line. Discrete measured or calculated WCT predictor values derived from paired baseline and WCT ECGs are integrated into the amplitude based WCT Formula to calculate VT probability (P_{VT}).

A calculation series is used to quantify the degree of QRS amplitude change that manifests between the baseline ECG and WCT event by converting raw ECG measurements into the frontal and horizontal PAC. The measured amplitudes (μV) of QRS waveforms above (+) (r/R and r′/R′) and below (−) (q/QS, s/S, and s′/S′) the isoelectric baseline from select frontal (aVR, aVL, aVF) and horizontal (V1, V4, V6) ECG leads were used to derive each calculation. Calculations were computed using JMP Pro 10 statistical software. Baseline Amplitude (BA), Absolute Amplitude Change (AAC) and Percent Amplitude Change (PAC) were calculated for both the frontal and horizontal ECG planes.

APC_{LeadX}=|(+)Amplitude_{WCT:LeadX}−(+)Amplitude_{Baseline:LeadX}|

ANC_{LeadX}=|(−)Amplitude_{WCT:LeadX}−(−)Amplitude_{Baseline:LeadX}|

TAC_{LeadX}=APC_{LeadX}+ANC_{LeadX}

TBA_{Baseline:LeadX}=(−)Amplitude_{Baseline:LeadX}+(+)Amplitude_{Baseline:LeadX}

where: LeadX denotes V1, V4, V6 (horizontal plane) or aVL, aVR, aVF (frontal plane). Note that (−) Amplitude=q/QS+s/S+s′/S′ and (+) Amplitude=r/R+r′/R′. Note that ANC and APC equations have the “absolute” mathematical annotation (e.g. |equation's contents|).

Absolute Amplitude Change (AAC) represents the absolute summative difference in QRS amplitude between the WCT and baseline ECG.

Frontal AAC=TAC_{aVR}+TAC_{aVL}+TAC_{aVF}

Horizontal AAC=TAC_{V1}+TAC_{V4}+TAC_{V6}

Baseline Amplitude (BA) represents the total sum amplitude of (+) and (−) QRS waveforms in the baseline ECG.

Frontal BA=TBA_{aVR}+TBA_{aVL}+TBA_{aVF}

Horizontal BA=TBA_{V1}+TBA_{V4}+TBA_{V6}

Percent Amplitude Change (PAC) represents the percent change in QRS amplitude between the WCT and baseline ECG.

$$Frontal PAC ( \% ) = ( \frac{Fr ontal AAC}{Frontal BA} ) \times 100$$

$$Horizontal PAC ( \% ) = ( \frac{Horizontal AAC}{Horizontal BA} ) \times 100$$

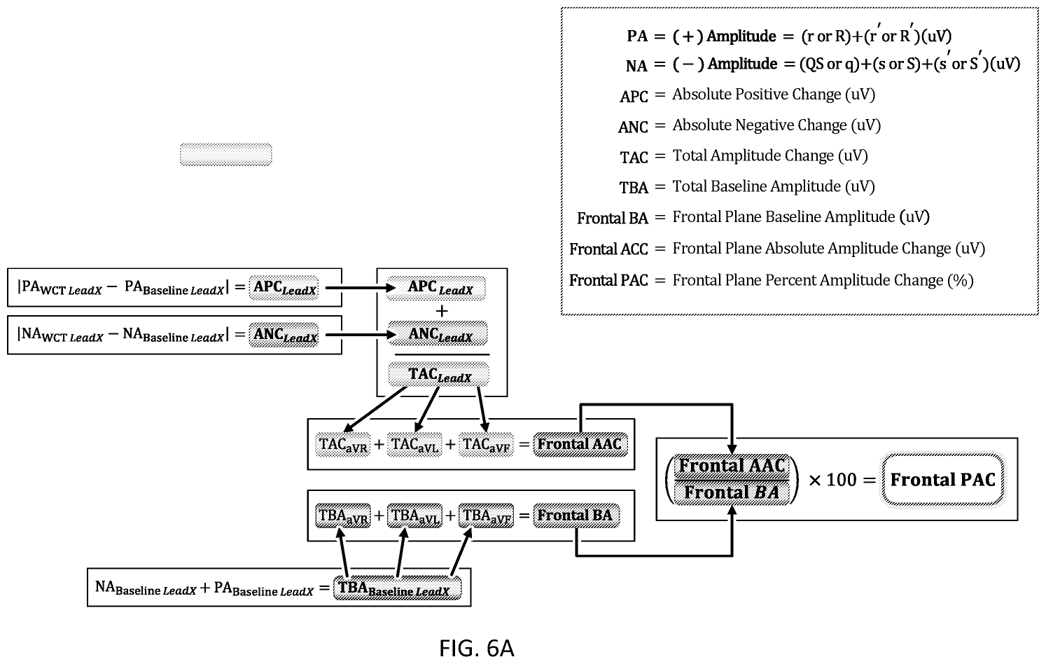

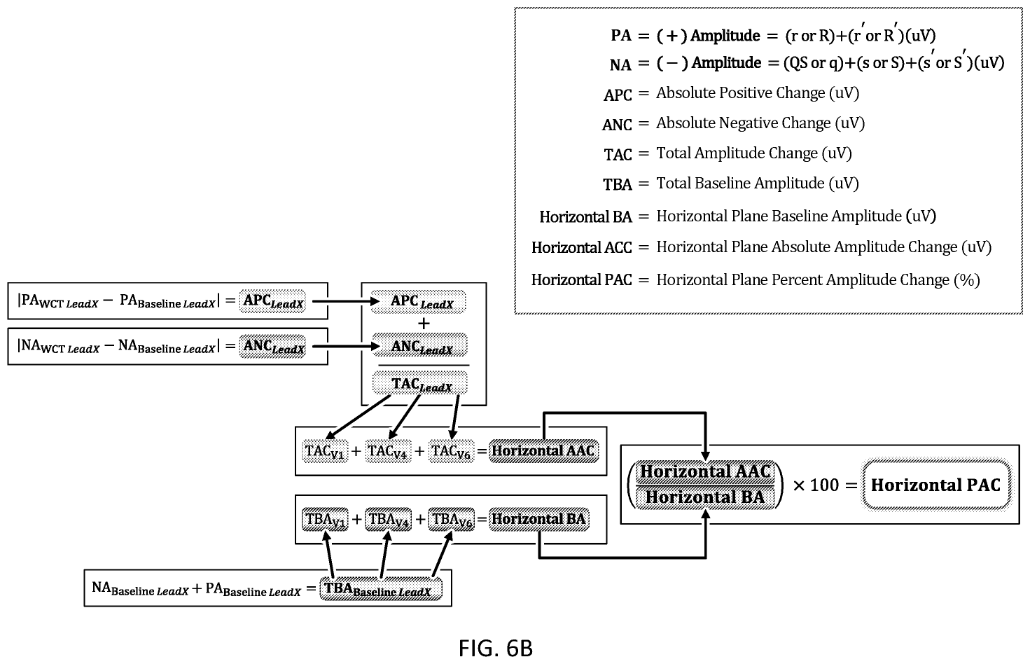

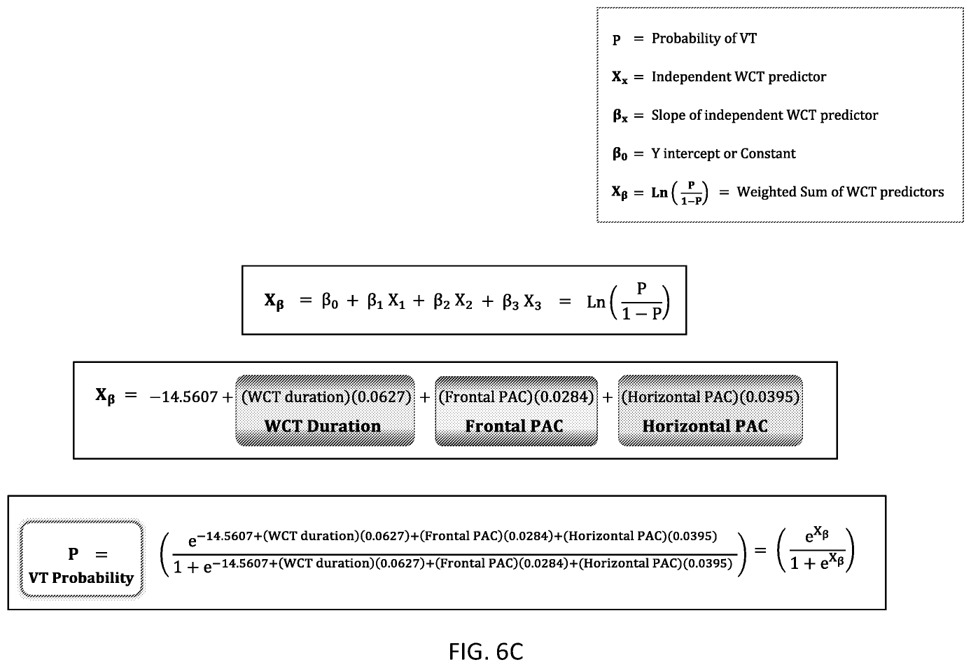

A diagram showing the derivation of the frontal PAC formula and horizontal PAC formula are shown in FIGS. 6A and 6B, respectively.

As previously mentioned, the amplitude based WCT Formula is a binary outcome logistic regression model that uses select independent WCT predictors: (1) WCT duration (ms), (2) frontal PAC (%) and (3) horizontal PAC (%). Each WCT predictor (Xₓ) was assigned beta coefficients (βₓ) according to their influence on the binary outcome (VT vs. non-VT). The “constant” term (B₀) represents the y-intercept of the least squares regression line. Discrete measured or calculated WCT predictor values derived from paired baseline and WCT ECGs are incorporated into the WCT Formula to calculate VT probability (P).

$${X}_{β} = ln ( \frac{{P}_{V T}}{1 - {P}_{V T}} ) = {β}_{0} + {β}_{1} {X}_{1} + {β}_{2} {X}_{2} + {β}_{3} {X}_{3} .$$

where: X_{β} is the weighted sum of the WCT predictors;

P_{VT} is the probablity of VT;

β₀ is the Y intercept or constant;

βₙ is the slope of the independent WCT predictor n;

Xₙ is the independent WCT predictor n; and

independent WCT predictors n are WCT_{duration}, PAC_{frontal}, and PAC_{horizontal}

$${P}_{V T} = \frac{{e}^{{X}_{β}}}{1 + {e}^{{X}_{β}}} \\ {P}_{V T} = \frac{{e}^{( a + b \times {WCT}_{duration} + c \times {PAC}_{frontal} + d \times {PAC}_{horizontal} )}}{1 + {e}^{( a + b \times {WCT}_{duration} + c \times {PAC}_{frontal} + d \times {PAC}_{horizontal} )}}$$

where a, b, c and d are constants:

a=intercept=−14.5607;

b=WCT QRS duration=0.0627;

c=frontal % change in area=0.284; and

d=horizontal % change in area=0.0395.

$${P}_{V T} = \frac{{e}^{( - 14.5607 + 0.0627 \times {WCT}_{duration} + 0.284 \times {PAC}_{frontal} + 0.0395 \times {PAC}_{horizontal} )}}{1 + {e}^{( - 14.5607 + 0.0627 \times {WCT}_{duration} + 0.284 \times {PAC}_{frontal} + 0.0395 \times {PAC}_{horizontal} )}} .$$

A diagram showing the amplitude based WCT Formula's logistic regression structure is shown in FIG. 6C.

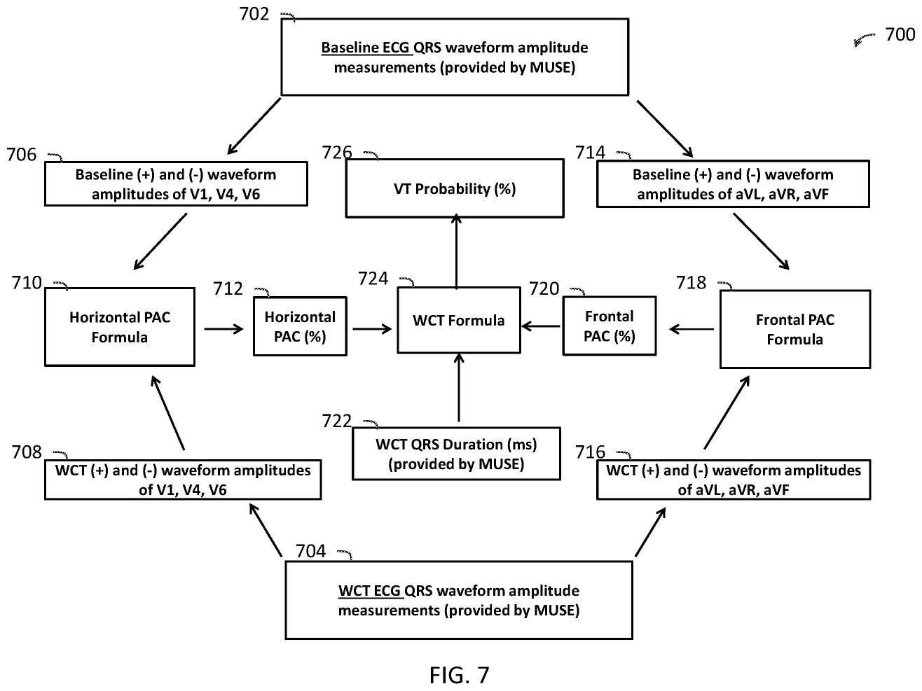

Referring now to FIG. 7, a flow diagram 700 representing the inputs and output of the amplitude based WCT Formula in accordance with one embodiment of the present invention is shown. The baseline ECG QRS waveform measurements may be obtained from GE Healthcare's MUSE or other computerized ECG interpretation software in block 702, and the WCT ECG QRS waveform measurements may be obtained from GE Healthcare's MUSE or other computerized ECG interpretation software in block 704. The baseline (+) and (−) waveform amplitudes of V1, V4, V6 in block 706 and WCT (+) and (−) waveform amplitudes of V1, V4, V6 in block 708 are used in the horizontal PAC formula in block 710 to provide the horizontal PAC (%) in block 712. The baseline (+) and (−) waveform amplitudes of aVL, aVR, aVF in block 714 and WCT (+) and (−) waveform amplitudes of aVL, aVR, aVF in block 716 are used in the frontal PAC formula in block 718 to provide the horizontal PAC (%) in block 720. The WCT QRS duration is provided in block 722, which is used along with the horizontal PAC (%) in block 712 and frontal PAC (%) in block 720 by the amplitude based WCT Formula in block 724 to provide the VT Probability (%) in block 726.

A two-part study was designed to build and validate the amplitude based WCT Formula capable of automatic VT probability estimation. In Part 1, a derivation cohort of paired WCT and subsequent baseline ECGs was used to construct a logistic regression model using the strongest independent predictors of VT and SWCT. Independent predictors including WCT QRS duration (ms), frontal ECG plane percent amplitude change (PAC) (%) and horizontal ECG plane percent amplitude change (PAC) (%) were incorporated into the amplitude based WCT Formula. In Part 2, the amplitude based WCT Formula's performance was prospectively tested using a separate validation cohort of paired WCT and subsequent baseline ECGs.

Paired WCT and subsequent baseline ECGs were derived from the Mayo Clinic Rochester and affiliated hospitals between September 2011 and November 2016. All ECGs were 12-lead recordings using standard paper speed (25 mm/s) and amplification (10 mm/mV). Electrocardiogram pairs were identified using a MUSE ECG databank system (GE Healthcare). Electrocardiograms fulfilling WCT criteria (QRS duration≥120 ms, heart rate ≥100 bpm) plus an ECG laboratory interpretation diagnosis of (1) “ventricular tachycardia,” (2) “supraventricular tachycardia,” or (3) “wide complex tachycardia” were defined as WCT events. Baseline ECGs were defined as the most proximate non-WCT ECG obtained after the WCT event. Electrocardiogram pairs were excluded if the WCT did not have a subsequent baseline ECG or definite clinical diagnosis recorded within the patient's electronic medical record. Polymorphic VTs and irregular SWCTs with varying atrioventricular (AV) conduction were excluded. Abbreviated WCTs that were not the dominant rhythm featured on the 12-lead ECG were excluded. Electrocardiogram pairs found to have irreconcilable faulty measurements (eg. QRS amplitude measurement of a pacing spike) or alternative lead placements (eg. right-sided chest leads) were excluded.

This version of the WCT Formula was developed and tested using two cohorts. The derivation cohort consisted of 328 paired WCT (160 VT, 168 SWCT) and baseline ECGs from 229 patients presenting to the Mayo Clinic Rochester (September 2011-March 2015). The validation cohort was comprised of 313 paired WCT (123 VT, 190 SWCT) and baseline ECGs from 228 patients presenting to the Mayo Clinic Rochester and/or Mayo Clinic Health System of South Eastern Minnesota—including 40 additional patient care locations: community hospitals, emergency departments, and outpatient clinics (April 2015-November 2016).

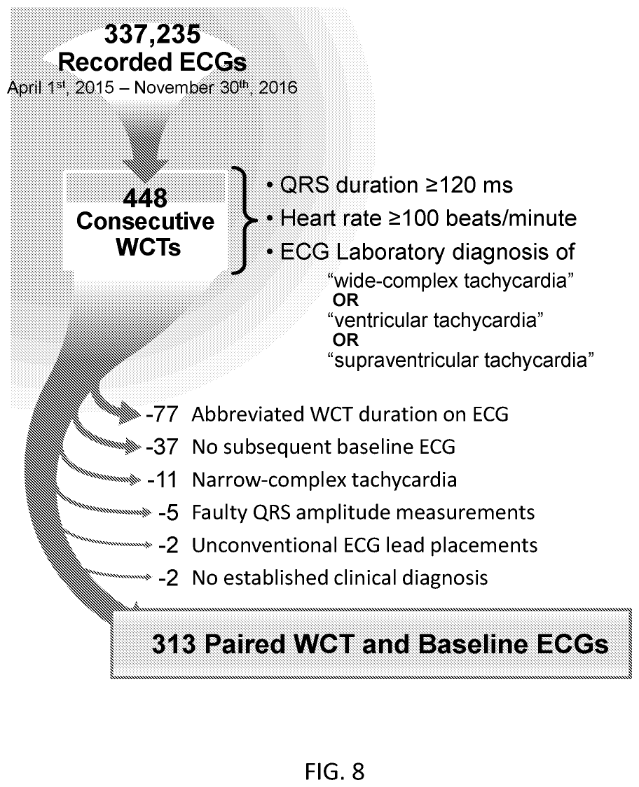

As shown in FIG. 8, various ECG pairs were excluded during validation cohort selection. Of the 337,235 recorded ECGs between Apr. 1, 2015 and Nov. 30, 2016, 448 consecutive WCTs were found that had a QRS duration greater than or equal to 120 ms, a heart rate greater than or equal to 100 beats/min and a ECG laboratory diagnosis of WCT or VT or SVT. One-hundred thirty-five out of 448 consecutive WCTs were excluded. More specifically, seventy-seven abbreviated WCTs that were not the dominant rhythm featured on the 12-lead ECG were excluded. Thirty-seven WCTs were excluded because there was no subsequent baseline ECG. Eleven WCTs demonstrated inappropriately prolonged QRS duration measurements for narrow complex SVTs. Five of the ECG pairs were excluded because of faulty QRS amplitude measurements of ventricular assist device artifact (n=1) or pacing spikes (n=4). Two of the ECG pairs were excluded due to unconventional ECG lead placements (i.e., right-sided chest leads). Two of the ECG pairs were exempted because they did not have an established clinical diagnosis. As a result, 313 paired WCT and baseline ECGs were used in the analysis

All selected ECGs were formally interpreted at the Mayo Clinic ECG laboratory. ECG interpretation was under the supervision of a rotating consortium of attending cardiologists and electrophysiologists. Each supervising interpreter possessed extensive ECG interpretation experience along with complete access to the patient's electronic medical record (including archived 12-lead ECGs). The interpretation strategy(s) utilized for each WCT was up to the supervising interpreter's discretion. The degree of diagnostic certainty reported by the ECG laboratory for each WCT was semi-qualitatively re-categorized: (1) definite VT, (2) probable VT, (3) definite SWCT, (4) probable SWCT and (5) undifferentiated. The time separation between the WCT and subsequent baseline ECG was recorded.

The patient's clinical diagnosis (VT or SWCT) was identified from the electronic medical record. The medical providers responsible for WCT diagnoses were categorized according to their level of expertise: (1) heart rhythm cardiologist, (2) non-heart rhythm cardiologist and (3) non-cardiologist. The final WCT rhythm diagnosis was determined by the patient's “most experienced” overseeing medical provider (heart rhythm cardiologist >non-heart rhythm cardiologist >non-cardiologist). Each diagnosing provider had access to the ECG laboratory's formal WCT interpretation. The completion of an electrophysiology procedure supporting the clinical diagnosis was recorded.

Clinical demographics including history of coronary artery disease (CAD), prior myocardial infarction (MI), prior cardiac surgery, congenital heart disease, cardiomyopathy (ischemic vs. non-ischemic), most proximate valuation of left ventricular ejection fraction (LVEF) (>=50%; 49-31%; <=30%), prior pacemaker or automatic implantable cardioverter-defibrillator (AICD) implantation, and ongoing Vaughan-Williams Class I and III antiarrhythmic drug (AAD) use were recorded from the electronic medical record.

Overall comparisons of continuous variables were completed using Wilcoxon rank-sum tests. Categorical variables were compared using Chi-square tests. Receiver operator curves were used to summarize selected independent continuous variables. Select independent predictors of VT and SWCT identified in the derivation cohort were used to generate a logistic regression formula (i.e. amplitude based WCT Formula). Designate independent variables (1) WCT duration (ms), (2) frontal PAC (%) and (3) horizontal PAC (%) were assigned beta coefficients according to their influence on the binary outcome (VT vs. non-VT). The amplitude based WCT Formula assigned an estimated VT probability (0.000%-99.999%) for each ECG pair of the validation cohort. The diagnostic value of various VT probability partitions were evaluated according to their agreement with clinical diagnosis. A 50% VT probability partition (VT=>50%; SWCT<50%) was used to assess for the amplitude based WCT Formula's agreement with ECG laboratory interpretation and clinical diagnosis. Diagnoses rendered by various VT probability partitions were used to assess its diagnostic performance (e.g., accuracy, sensitivity, specificity, positive likelihood ratio, negative likelihood ratio). Kappa statistics were applied to describe the diagnostic agreement between (1) clinical diagnosis, (2) ECG laboratory interpretation and (3) the amplitude based WCT Formula's 50% VT probability partition. McNemar's test was used to test for differences among diagnostic standards. Statistical analyses were completed using SAS version 9.4.

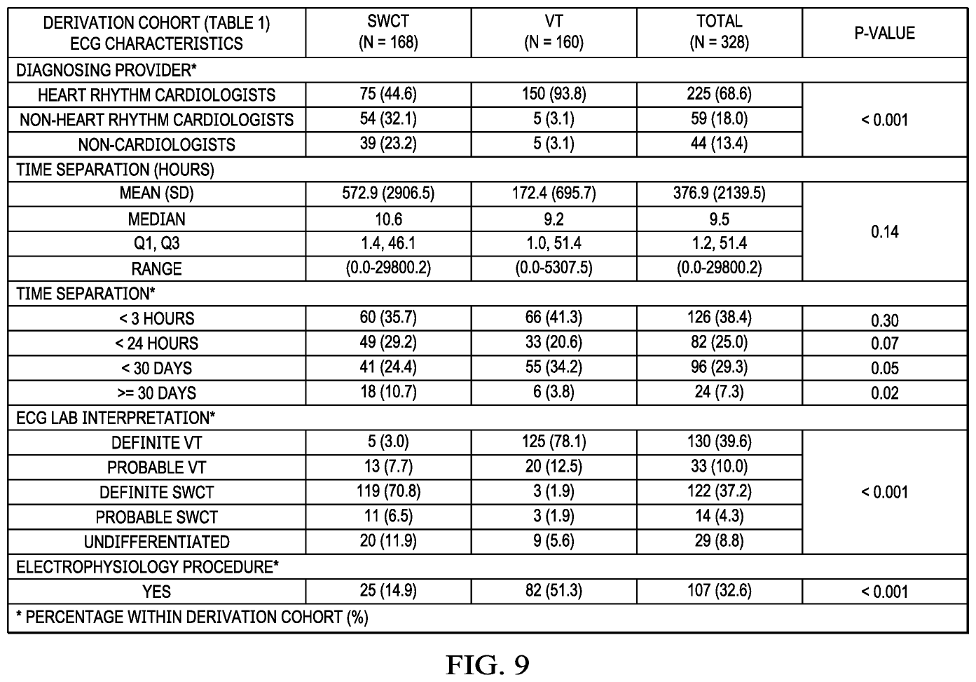

Now referring to FIG. 9, Table 1 shows the ECG characteristics of the derivation cohort, which consisted of 160 VTs and 168 SWCTs from 229 patients. The majority of clinical diagnoses were established by heart rhythm cardiologists or non-heart rhythm cardiologists (86.6%). The VT group had comparatively more clinical diagnoses established by heart rhythm cardiologists than the SWCT group (VT 93.8% vs. SWCT 44.6%, p<0.001). The SWCT group had a substantially higher percentage of clinical diagnoses established by non-cardiologists (SWCT 23.2% vs. VT 3.1%, p<0.001). The majority of WCTs were given definitive or probable interpretive diagnoses by the ECG laboratory (91.2%). Median time separation between the WCT event and subsequent baseline ECG was 9.5 hours. Most baseline ECGs were acquired within 24 hours of the WCT event (63.4%). Most clinical WCT diagnoses were not supplemented by the findings of an electrophysiology procedure (67.4%).

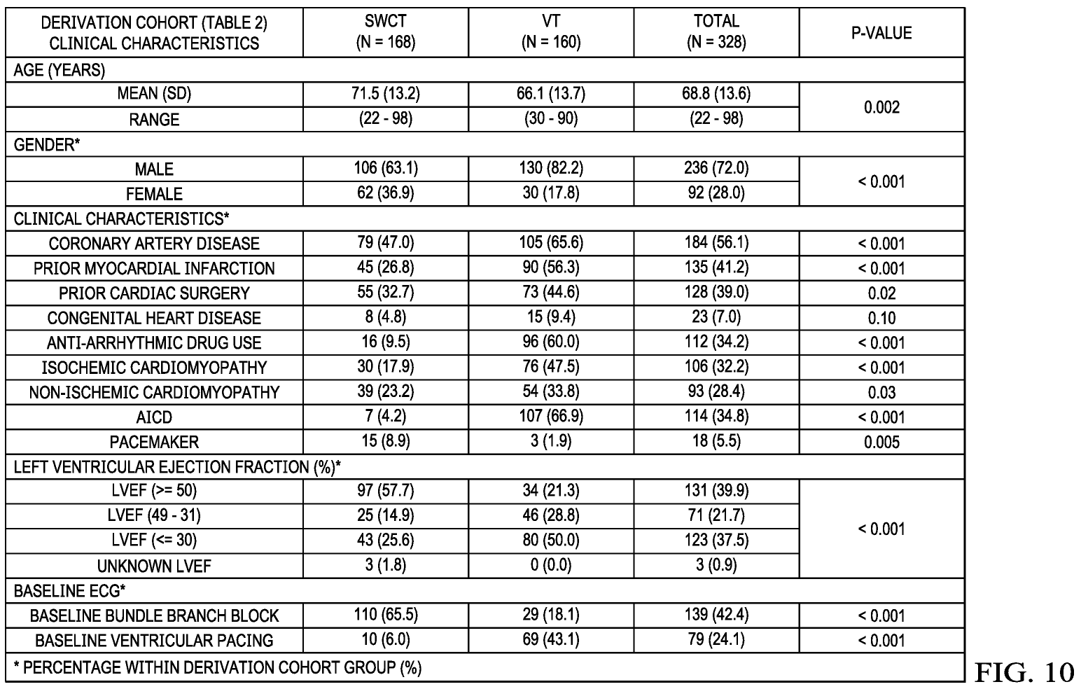

Referring now to FIG. 10, Table 2 shows the clinical characteristics of the derivation cohort. The majority of WCTs were derived from males (72.0%). The SWCT group included more events derived from females than the VT group (SWCT 36.9% vs. VT 17.8%, p<0.001). The average age of the VT group was 5.4 years younger than the SWCT group. The VT group included more events from patients with known CAD (p<0.001), prior MI (p<0.001), prior cardiac surgery (p=0.02), ongoing AAD use (p<0.001), ischemic cardiomyopathy (p<0.001), non-ischemic cardiomyopathy (p=0.03) and implanted AICD (p<0.001), while the SWCT group had more events from patients with pacemakers without defibrillator capability (p=0.005). The VT group possessed more events from patients with an LVEF<=30% (VT 50.0% vs. SWCT 25.6%, p<0.001), while the SWCT group had more events from patients with an LVEF>=50% (SWCT 57.7% vs. VT 21.3%, p<0.001). The SWCT group included more ECG pairs with baseline bundle branch block (BBB) (SWCT 65.5% vs. VT 18.1%, p<0.001). The VT group included more ECG pairs with baseline ventricular pacing (VT 43.1% vs. SWCT 6.0%, p<0.001).

Now referring to FIG. 11, Table 3 shows the ECG analysis for the derivation cohort. Significant differences between VT and SWCT groups were noted for baseline QRS duration (ms) (p=0.05), baseline QTc interval duration (ms) (p=0.05), WCT QRS duration (ms) (p<0.001), change in QRS duration (ms) (p<0.001), change in R wave axis (°) (p<0.001), change in T wave axis (°) (p<0.001), frontal PAC (%) (p<0.001) and horizontal PAC (%) (p<0.001).

The mean and proportional distribution of WCT QRS duration (ms) was greater in the VT group (SWCT 144.0 vs. VT 177.4, p<0.001) (FIG. 12A). Differences in WCT QRS duration were also appreciated among baseline ECG sub-groups: QRS duration >=120 ms (SWCT 144.3 vs. VT 180.6, p<0.001), QRS duration <120 ms (SWCT 143.1 vs. VT 171.1, p<0.001) and ventricular pacing (SWCT 157.2 vs. VT 187.2, p<0.001) (see FIG. 12D).

The mean and proportional distribution of frontal PAC (%) was greater in the VT group (SWCT 34.9 vs. VT 123.7, p<0.001) (FIG. 12B). Differences in frontal PAC were also appreciated among baseline ECG sub-groups: QRS duration >=120 ms (SWCT 30.9 vs. VT 127.5, p<0.001), QRS duration <120 ms (SWCT 47.0 vs. VT 116.5, p <0.001) and ventricular pacing (SWCT 61.9 vs. VT 135.8, p=0.004) (see FIG. 12D).

The mean and proportional distribution of horizontal PAC (%) was greater in the VT group (SWCT 44.2 vs. VT 116.0, p<0.001) (FIG. 12C). Differences in horizontal PAC were also appreciated among baseline ECG sub-groups: QRS duration >=120 ms (SWCT 39.7 vs. VT 109.0, p<0.001), QRS duration <120 ms (SWCT 57.9 vs. VT 129.3, p<0.001) and ventricular pacing (SWCT 49.2 vs. VT 123.6, p<0.001) (see FIG. 12D).

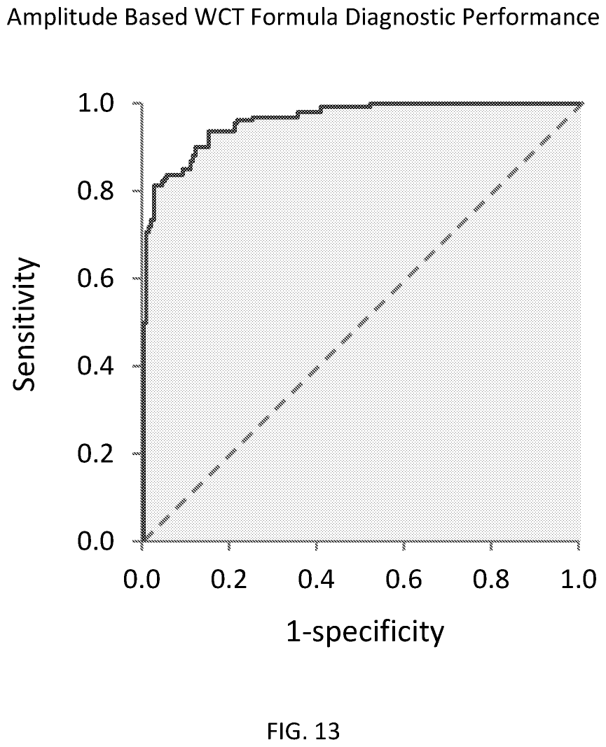

As shown in FIG. 11, WCT predictors included baseline QRS duration (ms) (p=0.05), baseline QTc interval duration (ms) (p=0.05), WCT QRS duration (ms) (p <0.001), change in QRS duration (ms) (p<0.001), change in R wave axis (°) (p<0.001), change in T wave axis (°) (p<0.001), frontal PAC (%) (p<0.001) and horizontal PAC (%) (p<0.001). As shown in FIG. 13, the amplitude based WCT Formula diagnostic performance including (1) WCT QRS duration (ms), (2) frontal PAC (%) and (3) horizontal PAC (%) demonstrated favorable VT and SWCT differentiation (AUC of 0.96) using the derivation cohort (collection of paired WCT and baseline ECGs).

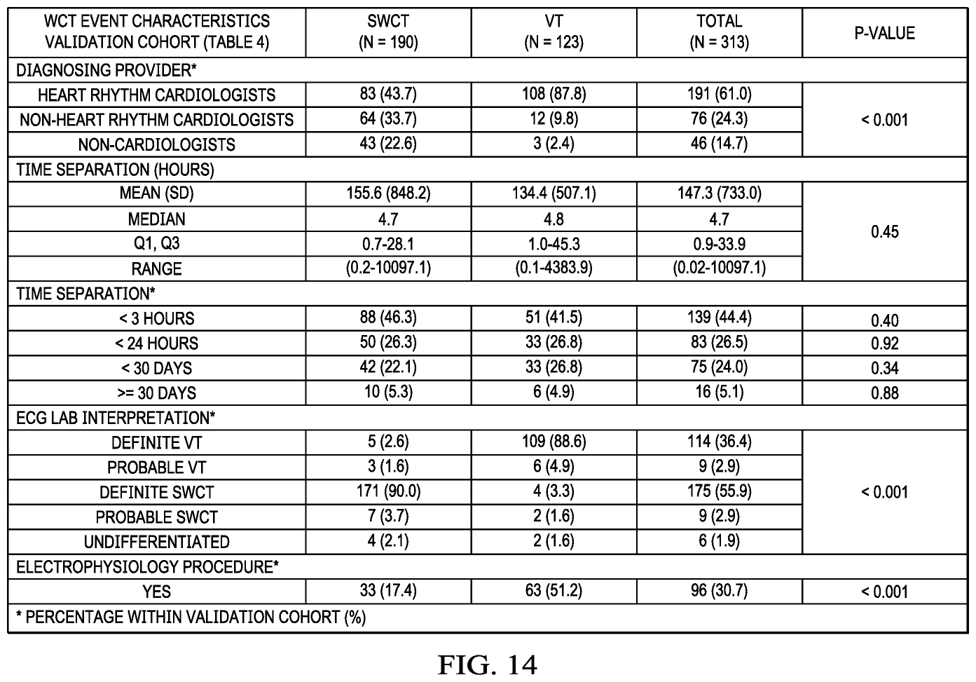

Referring now to FIG. 14, Table 4 shows the WCT event characteristics of the validation cohort, which consisted of 123 VTs and 190 SWCTs from 228 patients. The majority of clinical diagnoses were established by heart rhythm cardiologists or non-heart rhythm cardiologists (85.3%). The VT group had comparatively more clinical diagnoses established by heart rhythm cardiologists than the SWCT group (VT 87.8% vs. SWCT 43.7%, p<0.001). The SWCT group had a substantially higher percentage of clinical diagnoses established by non-cardiologists (SWCT 22.6% vs. VT 2.4%, p<0.001). The validation cohort included comparatively more WCTs with definitive or probable interpretive diagnoses coded by the ECG laboratory than the derivation cohort (98.1% vs. 91.2%, p<0.001). Median time separation between the WCT event and subsequent baseline ECG was 4.7 hours. Most baseline ECGs were acquired within 24 hours of the WCT event (70.9%). Most clinical WCT diagnoses were not supplemented by the findings of an electrophysiology procedure (69.3%).

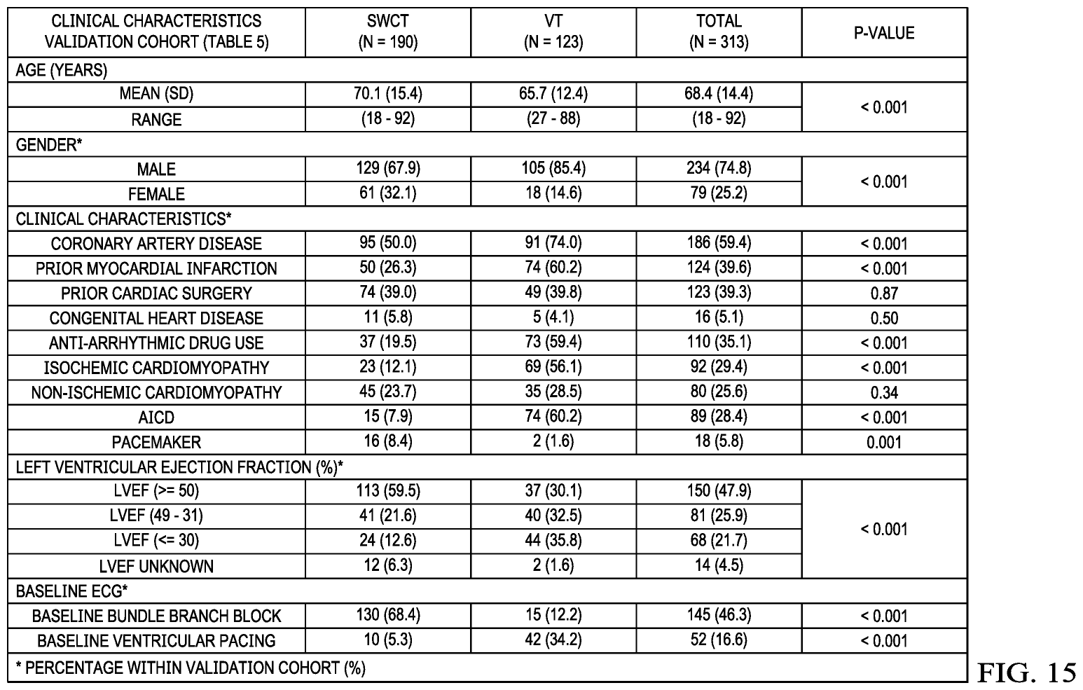

Now referring to FIG. 15, Table 5 shows the clinical characteristics of the validation cohort. The majority of WCTs were derived from males (74.8%). The SWCT group included more events derived from females than the VT group (SWCT 32.1% vs. VT 14.6%, p<0.001). The average age of the VT group was 4.4 years younger than the SWCT group. The VT group included more events from patients with known CAD (p<0.001), prior MI (p<0.001), ongoing AAD use (p<0.001), ischemic cardiomyopathy (p<0.001) and implanted AICD (p<0.001), while the SWCT group had more events from patients with pacemakers without defibrillator capability (p=0.01). The VT group possessed more events from patients with an LVEF<=30% (VT 35.8% vs. SWCT 12.6%, p<0.001), while the SWCT group had more events from patients with an LVEF>=50% (SWCT 59.5% vs. VT 30.1%, p<0.001). The SWCT group included more ECG pairs with baseline BBB (SWCT 68.4% vs. VT 12.2%, p<0.001). The VT group included more ECG pairs with baseline ventricular pacing (VT 34.2% vs. SWCT 5.3%, p<0.001).

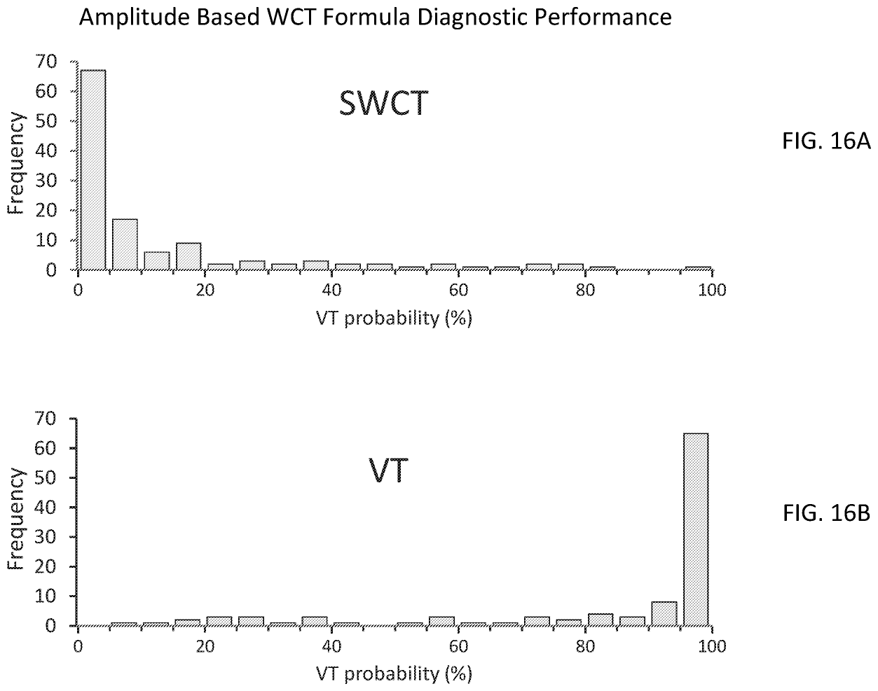

Referring now to FIGS. 16A and 16B, histograms demonstrating the distribution of clinical SWCT and VT for the validation cohort according to the amplitude based WCT Formula diagnostic performance at VT probability estimates (0.000%-99.999%) are shown in accordance with one embodiment of the present invention. Note that VT probability bins on the x-axis are arranged by 5.0% increments. Most VTs (77.2%) were categorized as having high VT probability (=>90.0%) with a compatible positive predictive value (97.9%). Most SWCTs (72.1%) were categorized as having low VT probability (<10.0%) with a compatible negative predictive value (97.2%).

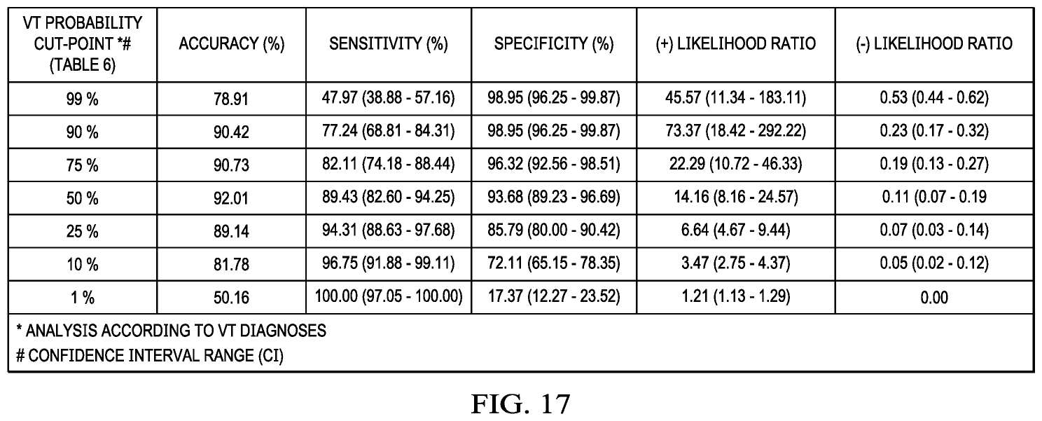

Now referring to FIG. 17, Table 6 shows the VT probability partitions in accordance with one embodiment of the present invention. This version of the WCT Formula demonstrated favorable diagnostic characteristics across a wide variety of VT probability partitions. A VT probability partition of 50% (VT>=50%; SWCT<50%) yielded strong overall accuracy (92.0%) with high sensitivity (89.4%) and specificity (93.7%). A VT probability partition of 25% (VT=>25%; SWCT<25%) yielded higher sensitivity (94.3%) with a minimal reduction in overall accuracy (89.1%) and specificity (85.8%).

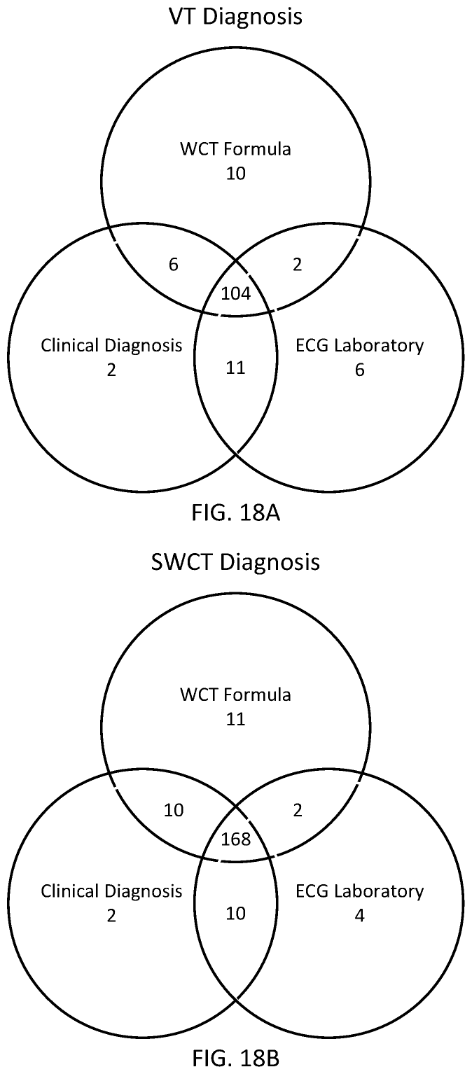

Referring now to FIGS. 18A and 18B, Venn diagrams summarizing the distribution of shared and non-shared VT (FIG. 18A) and SWCT (FIG. 18B) diagnoses established by three diagnostic standards: (1) clinical diagnosis, (2) ECG laboratory interpretation and (3) amplitude based WCT Formula's 50% VT probability cut-point are shown. Wide complex tachycardias without definitive VT or SWCT diagnoses coded by the ECG laboratory (ie. undifferentiated) were not included (n=6). The distribution of (1) clinical diagnoses, (2) ECG laboratory interpretations and (3) amplitude based WCT Formula diagnoses according to a VT probability cut-point of 50% reveals strong agreement between each diagnostic standard. The amplitude based WCT Formula's agreement with either or both ECG laboratory interpretation and clinical diagnosis for VT diagnoses was 91.1% and 84.6%, respectively. The amplitude based WCT Formula's agreement with either or both ECG laboratory interpretation and clinical diagnosis for SWCT diagnoses was 94.7% and 88.4%, respectively. The degree of agreement between each diagnostic standard for VT diagnoses was strong: (1) WCT Formula vs. ECG laboratory (κ=0.78, CI 0.71-0.85), (2) WCT Formula vs. clinical diagnosis (κ=0.83, CI 0.77-0.90) and (3) clinical diagnosis vs. ECG laboratory (κ=0.89, CI 0.84-0.94). Similarly, the degree of agreement between each diagnostic standard for SWCT diagnoses was strong: (1) WCT Formula vs. ECG laboratory (κ=0.72, CI 0.65-0.80), (2) WCT Formula vs. clinical diagnosis (κ=0.83, CI 0.77-0.90) and (3) clinical diagnosis vs. ECG laboratory (κ=0.85, CI 0.79-0.91). The WCT Formula and ECG laboratory did not differ in their degree of agreement with clinical diagnosis (p=0.86).

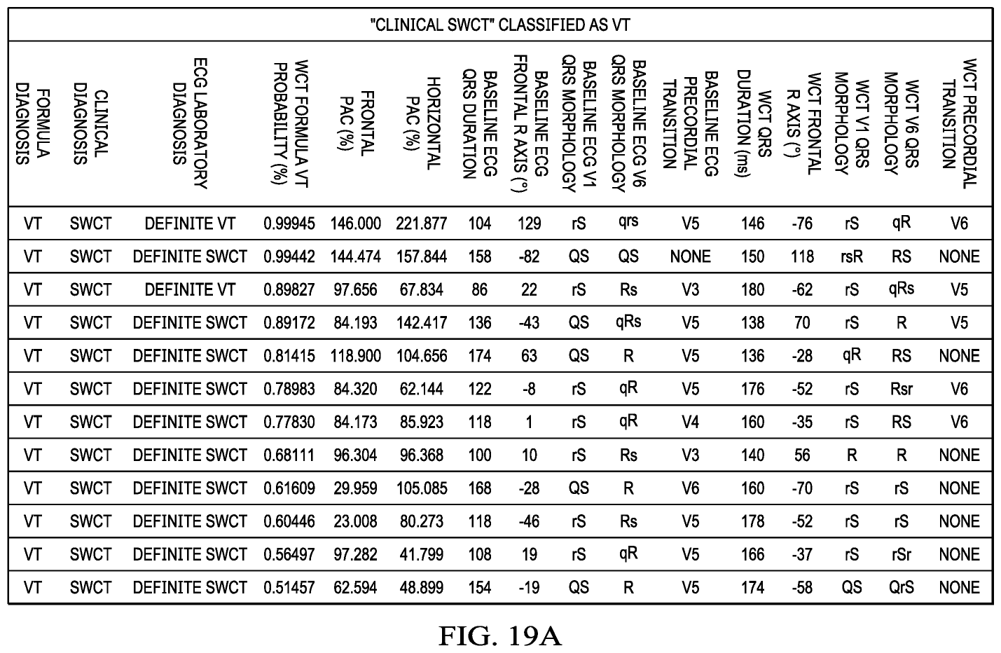

As show in in FIG. 19A, thirteen out of 123 (10.6%) “clinical VTs” were categorized as SWCT using the amplitude based WCT Formula's 50% VT probability partition −6 expressed a QRS duration <140 ms; 10 demonstrated a frontal plane QRS axis shift <40°; 4 exhibited an unchanged QRS configuration at lead V1; 3 exhibited an unchanged QRS configuration at lead V6.

As shown in FIG. 19B, twelve out of 190 (6.3%) “clinical SWCTs” were categorized as VT using the amplitude based WCT Formula's 50% VT probability partition −7 expressed a QRS duration=>160 ms; 9 demonstrated a frontal plane QRS axis shift >=40°; 5 exhibited QRS morphology changes at lead V1; 12 exhibited QRS morphology differences at lead.

This version of the WCT Formula accurately predicted the vast majority of WCTs in a prospective evaluation using paired WCT and baseline ECGs derived from clinical practice. Approximately 75% of WCTs from the validation cohort were accurately allocated as having high (=>90%) or low (<10%) VT probability. Additionally, the amplitude based WCT Formula's 50% and 25% VT probability partitions yielded favorable overall accuracy with strong sensitivity and specificity for VT.

The amplitude based WCT Formula's diagnoses agreed strongly with those provided by our institution's clinical diagnosis and ECG laboratory interpretation practices. Remarkably, despite the ECG laboratory's presumably strong influence on patients' final clinical diagnosis, the amplitude based WCT Formula was able to “match” the ECG laboratory's agreement with clinical diagnosis.

The amplitude based WCT Formula's 50% VT probability partition did not match the exceptional performance originally ascribed to the Brugada algorithm (accuracy 98.0%; sensitivity 98.7%; specificity 96.5%) or Lead II R-wave to peak time (RWPT) criterion (sensitivity 93.2%; specificity 99.3%) (6, 14). When compared to results first reported for Vereckei's lead aVR algorithm (12), the amplitude based WCT Formula's 50% VT probability cut-point appears to be less sensitive (lead aVR 96.5% vs. WCT Formula 89.4%) but more specific (lead aVR 75.0% vs. WCT Formula 93.7%). However, the amplitude based WCT Formula compares quite favorably to these other methods when they were appraised by independent authors (12, 15, 21-28). In general, independent studies that have aimed to validate these manual methods have found that they typically misdiagnose 15-30% of evaluated WCTs. One emblematic study which compared five different methods (Brugada, Griffith, Bayesian, lead aVR, and RWPT) in a head-to-head fashion found that they achieved only moderate diagnostic accuracy (range 68.8%-77.5%)(25). Each method, aside from the RWPT criterion, demonstrated good sensitivity (range 87.1%-94.2%) but poor specificity (range 39.8%-59.2%) for VT. Contrariwise, the RWPT criterion was found to be non-sensitive (60.0%) and moderately specific (82.7%).

It is well understood that the success of contemporary, manually-applied algorithms or criteria is highly dependent upon the examiner interpreting the ECG. It is also important to understand that most studies that have derived or validated manual WCT differentiation methods used only experienced electrocardiographers within controlled research settings (1-9, 11, 12, 14, 15, 17-20, 24, 25, 27, 29). Although some independent studies utilized less experienced ECG interpreters (21-23, 26, 28, 30), no study has attempted to test interpreter proficiency within authentic clinical settings.

Moreover, in clinical practice, it can be readily observed that the reliable differentiation of WCTs using 12-lead ECGs belongs only to knowledgeable providers who have a firm grasp of the advantages and disadvantages of multiple ECG criteria or algorithms, and are capable of their careful, systematic and simultaneous application. Apart from select cardiologists and electrophysiologists having an expertise in electrocardiography, this ability is not commonplace. Therefore, the efficacy of the published manual methods are likely less than what is reported due to their misapplication or failed utilization by less skilled ECG interpreters. This is especially likely when such clinicians are unexpectedly thrusted into the clinically challenging situation of managing a patient with WCT. The present invention, similar to any other automated diagnostic algorithm, does not suffer from these limitations.

The principal difference between the amplitude based WCT Formula and other established ECG interpretation methods is that it does not require manual ECG interpretation. Alternatively, the WCT Formula was designed to be automatically implemented by modern-day ECG interpretation software. Consequently, these methods escape the conventional challenges concerning provider recall (e.g., “What is the first step of the Brugada algorithm again?”), subjective interpretation (e.g., “Are those dissociated p waves?”), interobserver disagreement (21, 23, 26, 27, 30), and precise manual measurement (e.g., Vi/Vt of Vereckei's aVR algorithm) characteristically present among manual interpretation strategies (1-15). Instead, the WCT Formula provides an automatic and reliable means to differentiate WCTs irrespective of the user's ECG interpretation abilities. As a result, the WCT Formulas can help protect against or supersede faulty diagnoses reached by providers who incorrectly apply or fail to utilize manual interpretation methods.

The over-arching purpose of every proposed WCT differentiation criteria or algorithm is to help providers accurately differentiate WCTs. The preferred strategy utilized by most methods is an “absolute” rhythm classification (VT vs. SWCT) according to the presence or absence of select differentiation criteria (6, 8, 9, 11-14). While this approach is meant to lead clinicians to the correct WCT diagnosis, it often leaves providers unaware of the likelihood that their diagnosis is actually correct. This is because the published diagnostic sensitivity and specificity of the various ECG interpretation methods are usually not immediately available or remembered. Another drawback conventional WCT differentiation strategies is that they tend to overlook the predictive contributions of other relevant criteria found (or not found) on a patient's ECG.

The amplitude based WCT Formula was designed to simultaneously evaluate and precisely “weigh” multiple coexistent WCT predictors to provide an automatic estimation of VT probability. As a result, the amplitude based WCT Formula is able to deliver to its users an accurate and timely VT probability estimation to help them commit to or reconsider VT or SWCT diagnoses. Additionally, the WCT Formula's logistic regression structure can allow the incorporation of other ECG measurements or calculations that may help to differentiate WCTs.

Furthermore, after decades of research into manually-operated ECG criteria or algorithms, researchers still do not have a clear understanding of their overall practical value. This is partially explained by the fact that all published ECG interpretation methods utilized select patient populations referred for electrophysiology procedures to derive (3, 5, 6, 11, 12, 14, 15) or evaluate (4, 8-10, 18, 19, 21-27, 29) their respective criteria or algorithms. Although this strategy is quite justified as it helps confirm the veracity of WCT diagnoses, it consequently leads to an underrepresentation of WCTs diagnosed and managed non-invasively (e.g., SWCTs due to pre-existing aberrancy), as well as an over-representation of WCTs needing further evaluation and/or ablative therapies (e.g., idiopathic VTs or SWCTs due to pre-excitation). Furthermore, most prior studies either intentionally excluded or did not sufficiently report the inclusion of patients with pre-existing BBB (3, 6, 8, 10, 14, 18, 19, 21-23, 27), ongoing AAD use (3, 6-10, 14, 18, 21, 22, 27), congenital heart disease (3-10, 12, 14, 15, 19, 21-23, 27), idiopathic VTs (3, 6, 8, 14, 21, 22, 27), or pre-excited SWCTs (3, 5-8, 14, 19, 21-23, 27). This observation is particularly important because several established ECG methods have been shown to have reduced accuracy when applied to WCT populations including these various sub-groups (17, 20, 24-26, 29, 31).

In this study, approximately ⅔'s of WCTs did not have an accompanying electrophysiology procedure. As a result, the study population comprised many clinically encountered WCTs not customarily included in other studies. For example, the study cohorts included a higher percentage of patients with pre-existing BBB and ongoing AAD use than other studies (3-8, 10-12, 15, 18, 21-23, 25, 26). The SWCT groups were proportionally larger and included more events from patients with advanced age, CAD, prior MI and cardiomyopathy than other studies (4, 5, 11, 12, 15, 19, 23, 25, 26). Additionally, despite not being intentionally excluded, no pre-excited SWCTs were identified within the study cohorts. Although these dissimilarities primarily reflect the differing WCT selection strategy, they also indicate that the studies responsible for the derivation and evaluation of established ECG interpretation methods used select WCT populations different from what is regularly encountered in clinical practice.

According to the amplitude based WCT Formula's structure, “actual” VTs may be erroneously classified as SWCT if they demonstrate narrow QRS durations (e.g. fascicular VT) and/or very similar mean electrical vectors compared to the baseline ECG (e.g. bundle branch re-entry). Correspondingly, examples were observed where the amplitude based WCT Formula “missed VTs” with narrower QRS durations and/or similar QRS configurations compared to the baseline ECG (FIG. 19A). On the other hand, the amplitude based WCT Formula may erroneously classify “actual” SWCTs as VT if they express wider QRS durations (e.g. QRS prolongation due to ongoing antiarrhythmic drug use) and/or pronounced changes to the mean electrical vector (e.g. new left BBB aberrancy). Accordingly, we observed examples where the amplitude based WCT Formula erroneously predicted VT for clinical SWCTs exhibiting wider QRS durations and/or dissimilar QRS configurations compared to the baseline ECG (FIG. 19A).

The WCT Formula using time-voltage areas (ms/mV) of WCT ECG QRS waveforms instead of amplitudes will now be described. The included analyses pertaining to the time-voltage area based WCT Formula were derived from 641 paired WCT and baseline ECGs that constitute the summation of the derivation and validation cohorts for the amplitude based WCT Formula.

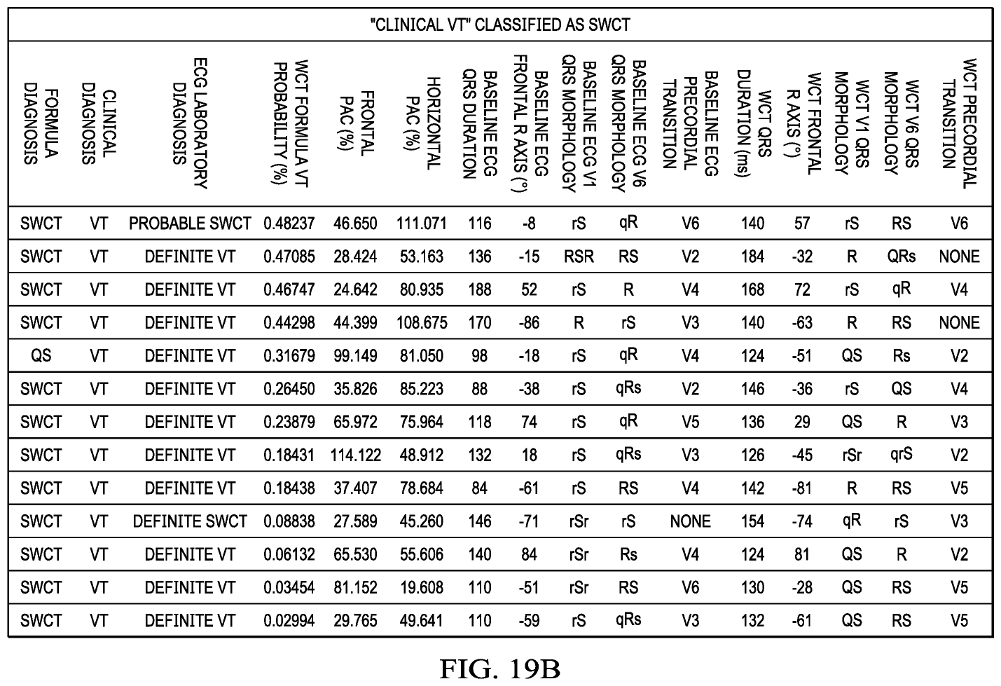

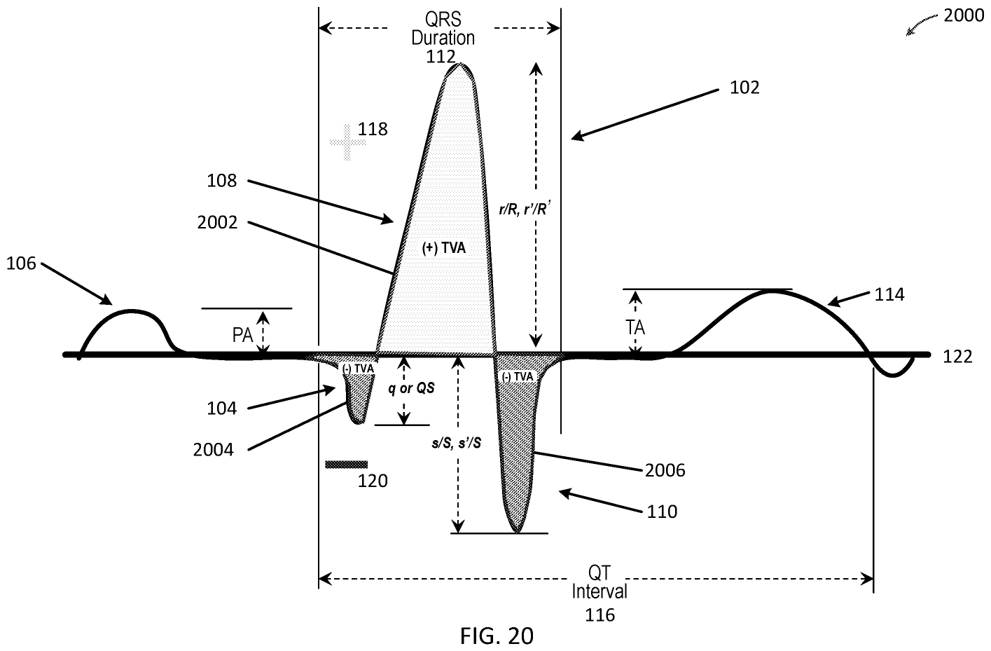

Now referring to FIG. 20, a schematic representation of a stereotypical ECG pattern for a single heart beat 2000 is shown. The various waves were previously described in reference to FIG. 1A and common reference numerals are used for both figures. Each wave has amplitude denoted as PA, q (QS not shown), r/R (r′/R′ not shown), or s/S (s′/S′ not shown) and TA. In addition, the QT interval 116 is the time interval extending from the onset of the QRS complex waveform 102 to the end of the T wave 114. The QRS complex 102 is divided into positive (+) time-voltage areas (TVA) 118 and negative (−) TVAs 120. The positive (+) TVAs 118 are the TVAs of the vertical QRS complex deflections above the isoelectric baseline 122, namely the TVAs of r/R wave (and r′/R′ not shown), wave 2002. The negative (−) TVAs 120 are the TVAs of the vertical QRS complex deflections below the isoelectric baseline 122, namely the TVAs of q (or QS wave not shown) 2004, and s/S (and s′/S′ not shown) wave 2006. Computerized ECG interpretation software, such as the MUSE software provided by GE Healthcare, automatically measures QRS complex waveform 102 attributes, namely q or QS, r/R, s/S, r′/R′, s′/S′ durations (ms), amplitudes (μV), and time-voltage areas (μV·ms). Note that standard annotation of QRS complex waveforms of small QRS waveforms are in lower case and large QRS waveforms are in upper case.

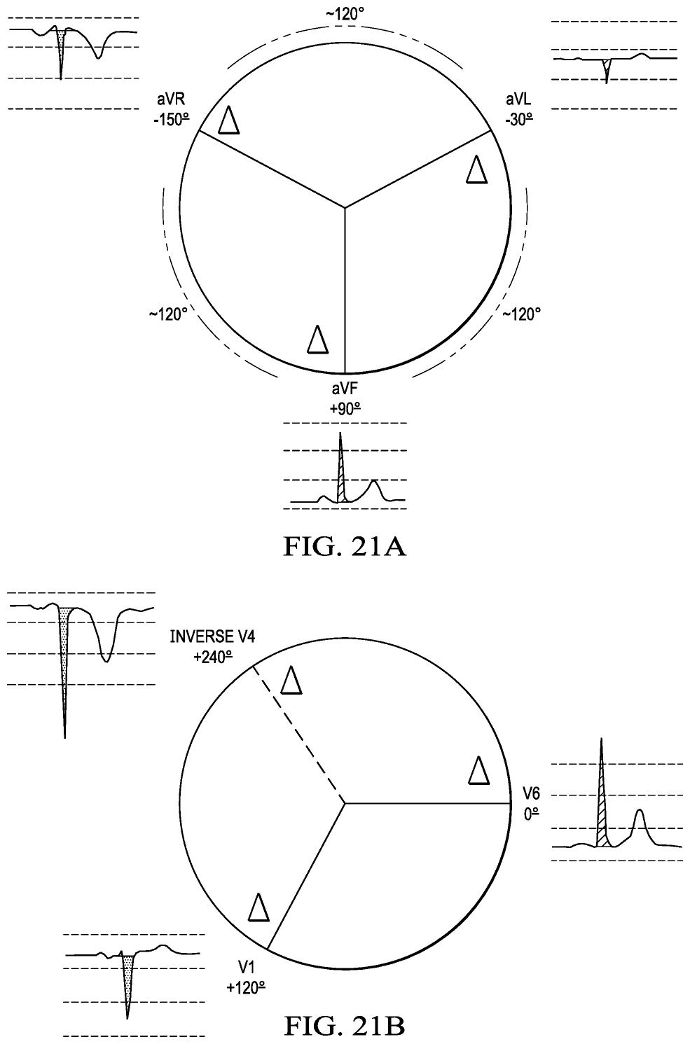

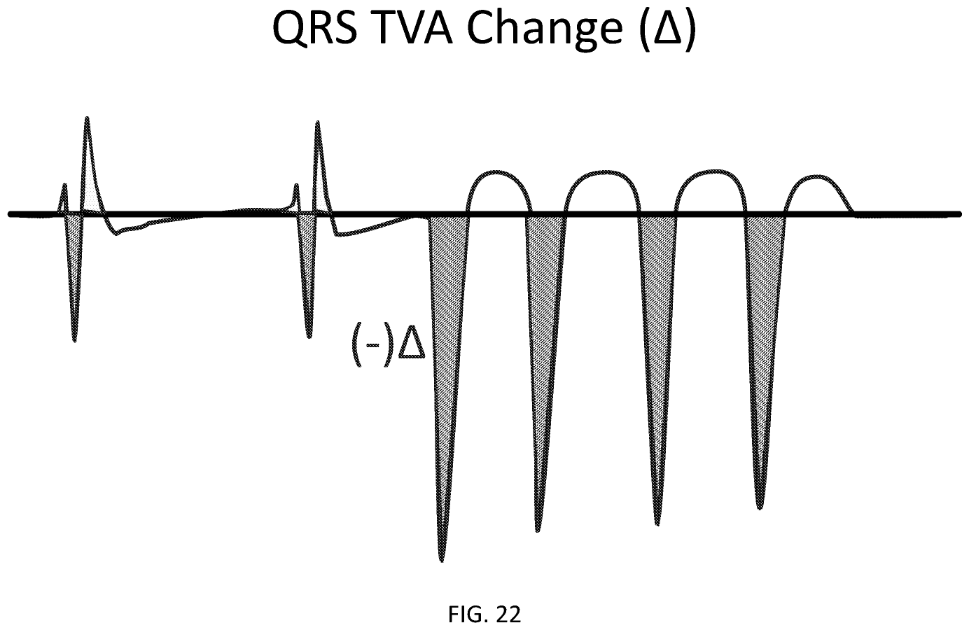

Referring now to FIGS. 21A-21B, graphic depictions of select ECG lead combinations utilized by the frontal (aVR, aVL, aVF)(FIG. 5A) and horizontal (V1, inverse V4, V6)(FIG. 5B) percent time-voltage area change (PTVAC) formulas in accordance with one embodiment of the present invention are shown. The QRS time-voltage area change (Δ) that manifests between the baseline and WCT ECGs at these selected leads is the foundation for each PTVAC calculation. Note that the absolute QRS time-voltage area changes (Δ's) that manifest in lead V4 are mathematically equivalent to its planar opposite: inverse V4. FIG. 22 depicts a schematic representation of the resultant QRS time-voltage area changes that manifest between a patient's baseline and WCT ECG.

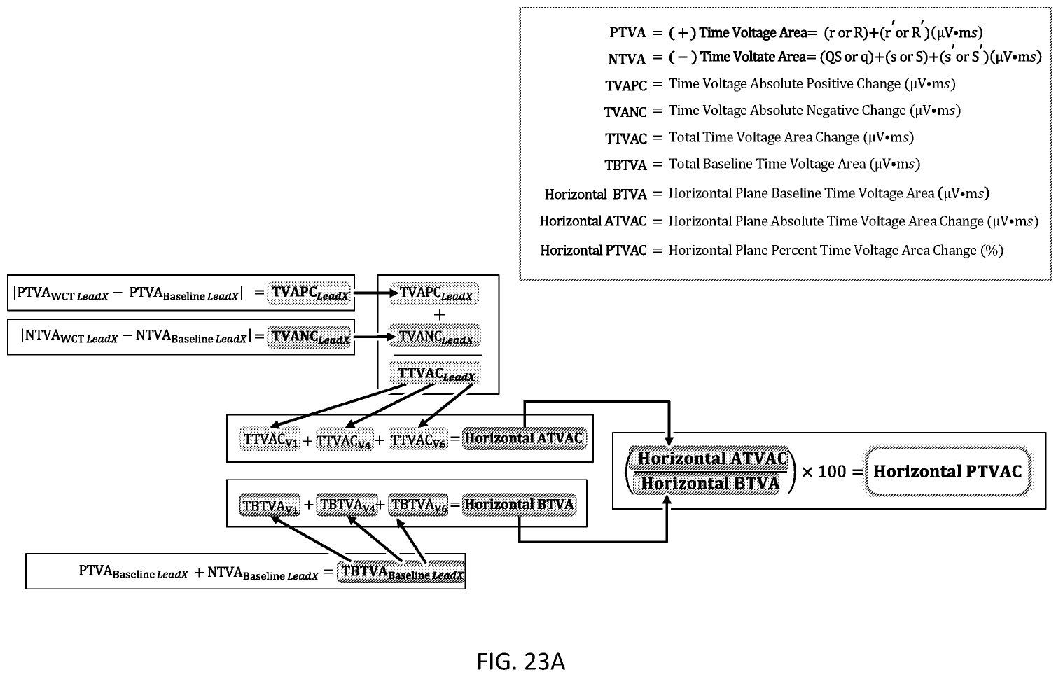

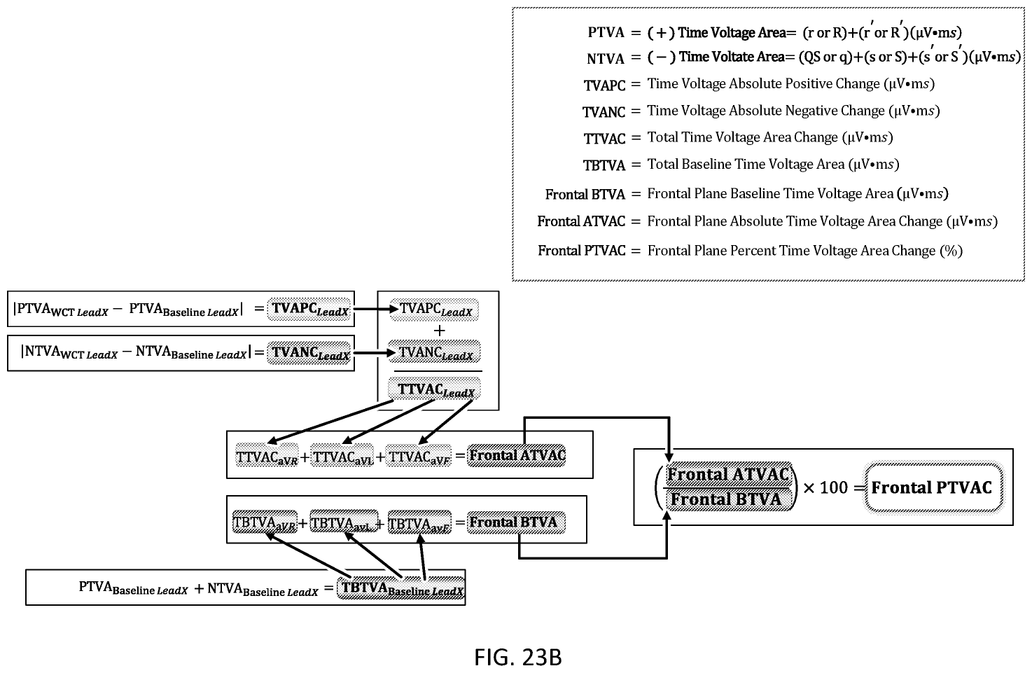

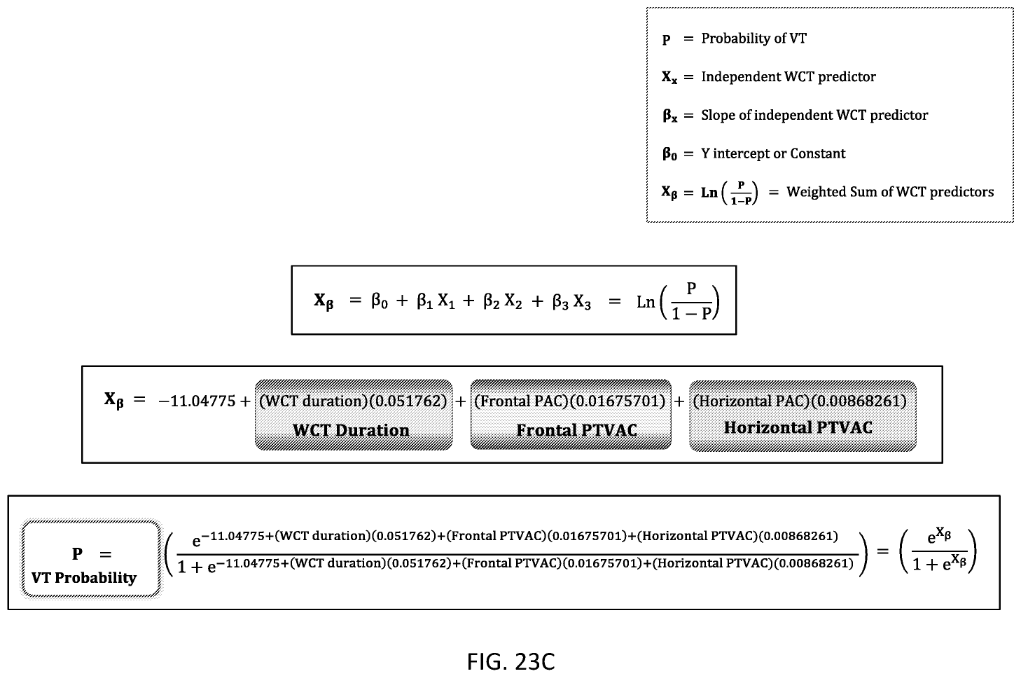

Now referring to FIGS. 23A-23B, the formulas for the horizontal PTVAC and frontal PTVAC are shown. The time-voltage area based WCT Formula incorporates the strong independent WCT predictors including (1) WCT QRS duration (ms), (2) frontal PTVAC (%) and (3) horizontal PTVAC (%). The predictive contribution of each WCT predictor is concomitantly “weighed” according to their influence on the binary outcome (VT vs. SWCT) to render a exact VT probability estimation. Given each WCT predictor's direct relationship with VT likelihood, the time-voltage area based WCT Formula estimates higher VT probabilities for ECG pairs demonstrating greater WCT QRS durations, frontal PTVAC and/or horizontal PTVAC. Similarly, the time-voltage area based WCT Formula estimates lower VT probability for ECG pairs with smaller WCT QRS durations, frontal PTVAC and/or horizontal PTVAC.

Similar to the amplitude based WCT Formula, the time-voltage area based WCT Formula is a multivariate logistic regression model that allows (1) delivery of precise VT probability predictions and (2) later inclusion of other well-established, enhanced and/or newly formulated WCT predictors. Other “machine learning” or artificial intelligence prediction methods (e.g., artificial neural networks, support vector machines, Random Forests, etc.) can be used with the frontal and horizontal PTVAC calculations. A step-wise decision-tree approach to diagnosis was avoided because of its tendency to prematurely commit to WCT diagnoses without considering the predictive strengths of other relevant predictors. The use of specific value cut-offs for VT diagnoses (e.g., QRS duration=>160 ms or frontal PAC>=75%) was avoided because they tend to cause (1) misclassifications due to VT and SWCT overlap and (2) ambiguity concerning the strength of WCT diagnoses for values distributed well above, well below, or at the margin of the designated cut-offs.

The time-voltage area based WCT Formula is a logistic regression formula that uses select independent WCT predictors (WCT QRS duration (ms), frontal PTVAC (%) and horizontal PTVAC (%)) to render a precise prediction of VT probability (%). Each WCT predictor (Xₓ) was assigned beta coefficients (βₓ) according to their influence on the binary outcome (VT vs. non-VT). The “constant” term (B₀) represents the y-intercept of the least squares regression line. Discrete measured or calculated WCT predictor values derived from paired baseline and WCT ECGs are incorporated into the time-voltage area based WCT Formula to calculate VT probability (P_{VT}).

A calculation series is used to quantify the degree of QRS time-voltage area change that manifests between the baseline ECG and WCT event by converting raw ECG measurements into the frontal and horizontal PTVAC. The measured time-voltage areas (μV·ms) of QRS waveforms above (+) (r/R and r′/R′) and below (−) (q/QS, s/S, and s′/S′) the isoelectric baseline from select frontal (aVR, aVL, aVF) and horizontal (V1, V4, V6) ECG leads were used to derive each calculation. Baseline Time-Voltage Area (BTVA), Absolute Time-Voltage Area Change (ATVAC) and Percent Time-Voltage Area Change (PTVAC) were calculated for both the frontal and horizontal ECG planes.

TVAPC_{LeadX}=|(+)TimeVoltageArea_{WCT:LeadX}−(+)TimeVoltageArea_{Baseline:LeadX}|

TVANC_{LeadX}=|(−)TimeVoltageArea_{WCT:LeadX}−(−)TimeVoltageArea_{Baseline:LeadX}|

TTVAC_{LeadX}=ATAPC_{LeadX}+TVANC_{LeadX}

$${TBTVA}_{Baseline : LeadX} = ( - ) {TimeVoltageArea}_{Baseline : LeadX} + ( + ) Tim {eVoltageArea}_{Baseline : LeadX}$$

where: LeadX denotes V1, V4, V6 (horizontal plane) or aVL, aVR, aVF (frontal plane). Note that (−) TVA=q/QS+s/S+s′/S′ and (+) TVA=r/R+r′/R′. Note that TVANC and TVAPC equations exhibit an “absolute” mathematical annotation (e.g. |equation's contents|).

Absolute Time-Voltage Area Change (ATVAC) represents the absolute summative difference in QRS time-voltage area between the WCT and baseline ECG.

Frontal ATVAC=TTVAC_{aVR}+TTVAC_{aVL}+TTVAC_{aVF}

Horizontal ATVAC=TTVAC_{V1}+TTVAC_{V4}+TTVAC_{V6}

Baseline Time-Voltage Area (BTVA) represents the total sum time-voltage area of (+) and (−) QRS waveforms in the baseline ECG.

Frontal BTVA=TBTVA_{aVR}+TBTVA_{aVL}+TBTVA_{aVF}

Horizontal BTVA=TBTVA_{V1}+TBTVA_{V4}+TBTVA_{V6}

Percent Time-Voltage Area Change (PTVAC) represents the percent change in QRS time-voltage area between the WCT and baseline ECG.

$$Frontal PTVAC ( \% ) = ( \frac{Fr ontal ATVAC}{Frontal BTVA} ) \times 100$$

$$Horizontal PTVAC ( \% ) = ( \frac{Horizontal ATVAC}{Horizontal BTVA} ) \times 100$$

As previously mentioned, the time-voltage area based WCT Formula is a binary outcome logistic regression formula that uses select independent WCT predictors: (1) WCT duration (ms), (2) frontal PTVAC (%) and (3) horizontal PTVAC (%). Each WCT predictor (Xₓ) was assigned beta coefficients (βₓ) according to their influence on the binary outcome (VT vs. non-VT). The “constant” term (B₀) represents the y-intercept of the least squares regression line. Discrete measured or calculated WCT predictor values derived from paired baseline and WCT ECGs are incorporated into the time-voltage area based WCT Formula to calculate VT probability (P).

$${X}_{β} = ln ( \frac{{P}_{V T}}{1 - {P}_{V T}} ) = {β}_{0} + {β}_{1} {X}_{1} + {β}_{2} {X}_{2} + {β}_{3} {X}_{3} .$$

where: X_{β} is the weighted sum of the WCT predictors;

P_{VT} is the probablity of VT;

β₀ is the Y intercept or constant;

βₙ is the slope of the independent WCT predictor n;

Xₙ is the independent WCT predictor n; and

independent WCT predictors n are WCT duration, PTVAC_{frontal}, and

$${PTVAC}_{horizontal}$$

$${P}_{VT} = \frac{{e}^{{X}_{β}}}{1 + {e}^{{X}_{β}}}$$

$${P}_{V T} = \frac{{e}^{( a + b \times {WCT}_{duration} + c \times {PTVAC}_{frontal} + d \times {PTVAC}_{horizontal} )}}{1 + {e}^{( a + b \times {WCT}_{duration} + c \times {PTVAC}_{frontal} + d \times {PTVAC}_{horizontal} )}} .$$

where a, b, c and d are constants:

a=intercept=−11.047775;

b=WCT QRS duration=0.051762;

c=frontal % change in time-voltage area=0.01675701; and

d=horizontal % change in time-voltage area=0.00868261.

$${P}_{V T} = \frac{{e}^{( - 11.047775 + 0.051762 \times {WCT}_{duration} + 0.01675701 \times {PTVAC}_{frontal} + 0.00868261 \times {PTVAC}_{horizontal} )}}{1 + {e}^{( - 11.047775 + 0.051762 \times {WCT}_{duration} + 0.01675701 \times {PTVAC}_{frontal} + 0.00868261 \times {PTVAC}_{horizontal} )}} .$$

A diagram showing the amplitude based WCT Formula's logistic regression structure is shown in FIG. 23C.

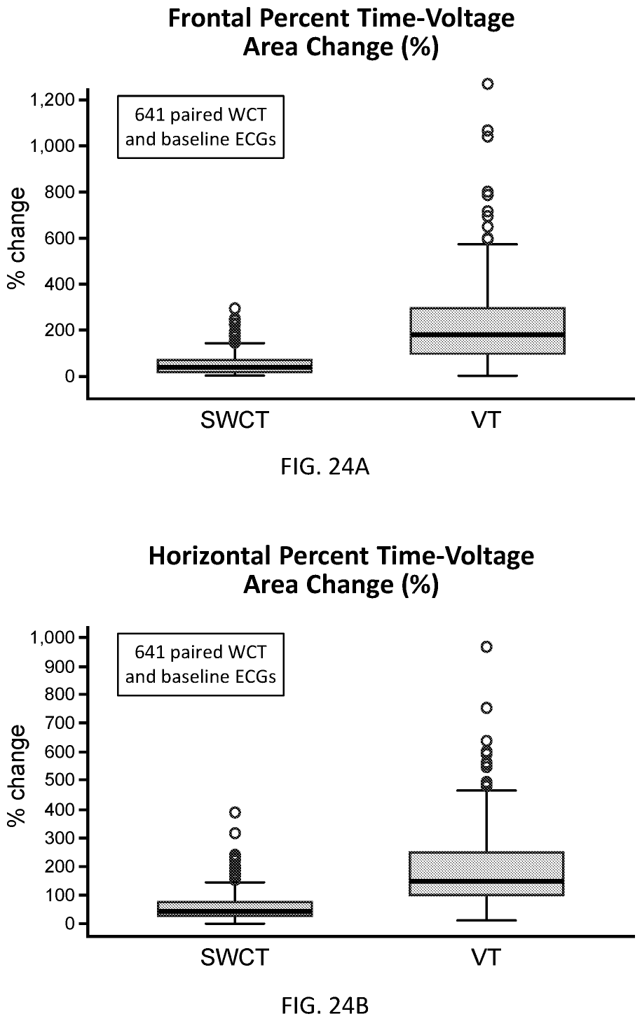

FIGS. 24A-24B are box-plots demonstrating the median and proportional distribution of frontal PTVAC (%) (FIG. 24A) and horizontal PTVAC (%) (FIG. 24B) for VT and SWCT groups in accordance with one embodiment of the present invention.

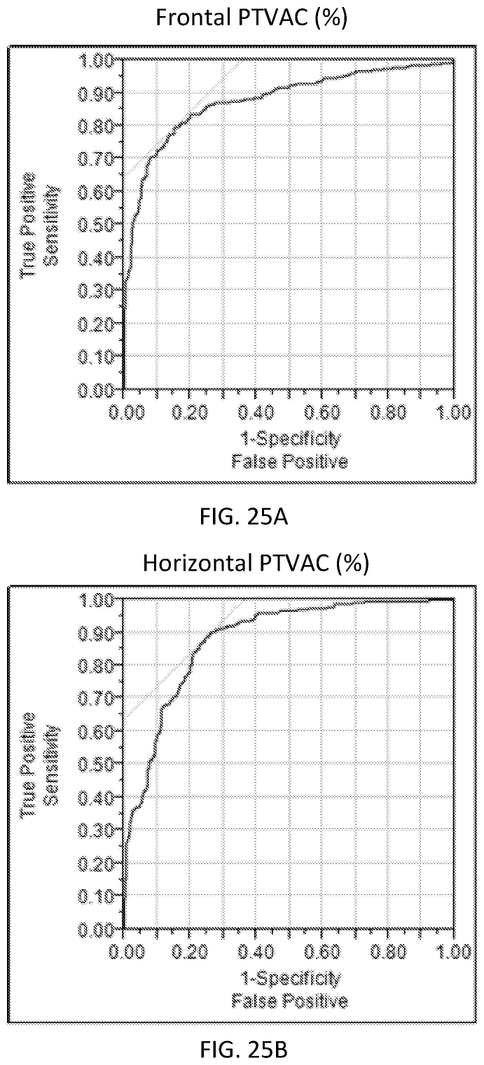

FIGS. 25A-25B are graphs depicting the frontal PTVAC (%) (FIG. 25A) and horizontal PTVAC (%) (FIG. 25B) in accordance with one embodiment of the present invention.

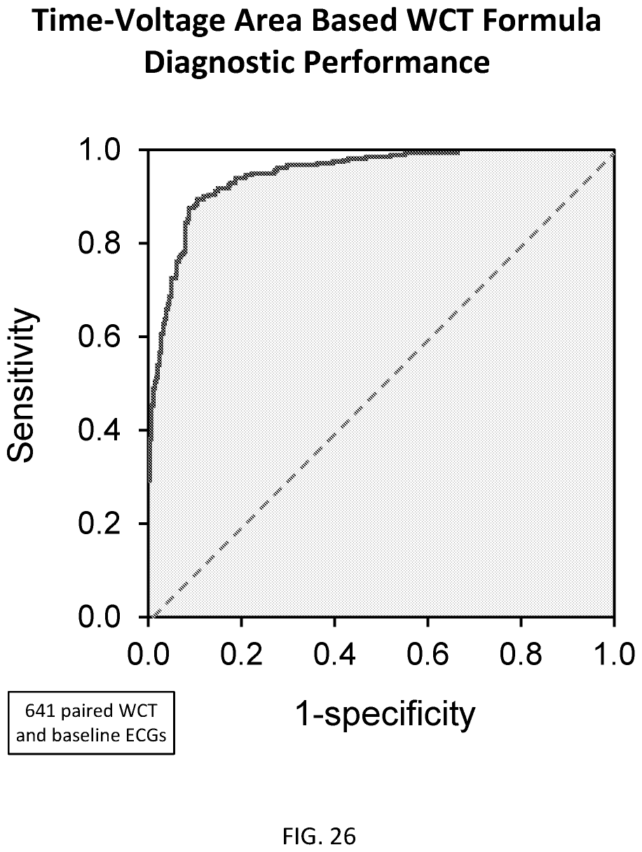

FIG. 26 is a graph depicting the time-voltage area based WCT Formula diagnostic performance (AUC of 0.95) in accordance with one embodiment of the present invention.

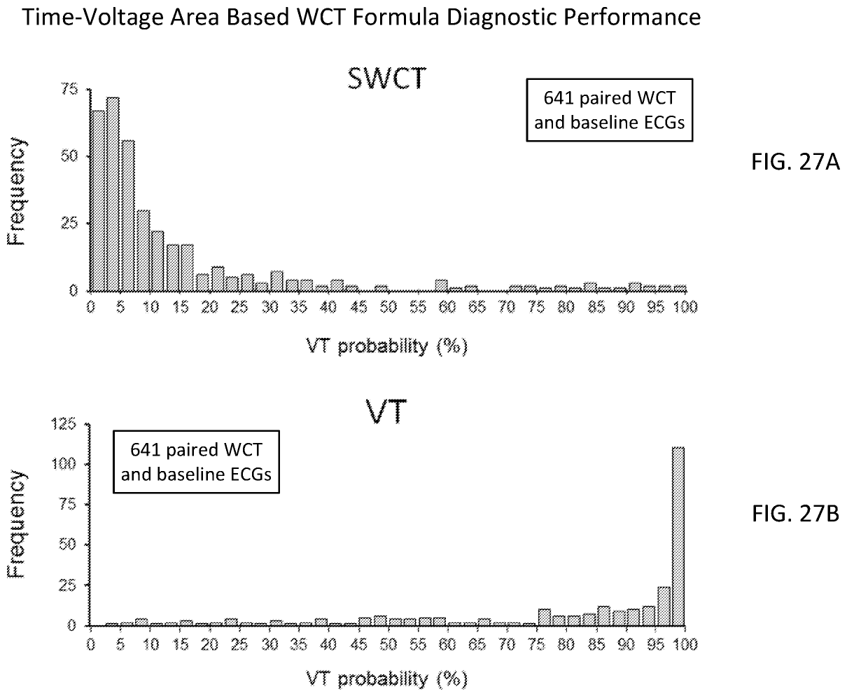

FIGS. 27A and 27B are histograms demonstrating the distribution of clinical VT probabilities according to the time-voltage area based WCT Formula diagnostic performance at VT probability estimates (0.000%-99.999%). The 641 paired WCT and baseline ECGs include both the validation and derivation cohorts.

The following discussion refers to both the amplitude and time-voltage area versions of the WCT Formula. The WCT Formulas rely upon the presumed accuracy of ECG software measurements. Moreover, the WCT Formulas require the simultaneous evaluation of the WCT and baseline ECG. Before the technological advances of ECG interpretation software and electronic databank storage systems, the automatic application of sophisticated computer algorithms using data from multiple ECGs was not feasible. Fortunately, contemporary ECG interpretation software is now able to simultaneously record, store and integrate data from multiple ECGs occurring before and after WCT events. Although the WCT Formulas' derivation and evaluation used only subsequent baseline ECGs, its performance is expected to be similar if applied baseline ECGs preceding the WCT event. For clinical situations where WCT patients present without previously recorded ECGs, providers will need to rely upon conventional ECG interpretation methods until they obtain the patient's baseline ECG.

The WCT Formulas were derived from paired WCT and baseline ECGs acquired from clinical practice. Included WCTs did not require electrophysiology testing for further diagnostic confirmation. Although this selection strategy helps to avoid selection biases, it does not “guarantee” the accuracy of WCT diagnoses established by the ECG laboratory and clinicians. Nor does it allow a more comprehensive understanding of the strengths and weaknesses of both WCT Formulas that would be accomplished with electrophysiology testing.

The WCT Formulas were derived and evaluated using paired WCT and baseline ECGs separated by varying, sometimes lengthy, time intervals. As a consequence, deviations in ECG electrode placement and/or major changes to the patient's baseline ECG (e.g. new ventricular pacing following AICD implantation) may have influenced study results.

It is expected that the WCT formulas, or its electrophysiological principles, can be used on not only for 12-lead ECGs, but for any extended heart rhythm monitoring devices such as continuous ECG telemetry monitors, stress testing systems, extended monitoring devices (e.g., Holter monitors, etc.), smartphone-enabled ECG medical devices, cardioverter-defibrillator therapy devices, such as wearable cardioverter defibrillators (e.g., Zoll Life Vest), subcutaneous implantable cardioverter defibrillators (S-ICD) (e.g., Emblem S-ICD by Boston Scientific), intracardiac or transvenous pacemaker devices, automated external defibrillators (AED) (e.g., HeartStart OnSite AED by Phillips), and conventional automatic implantable cardioverter defibrillators (AICD).

In the foregoing study, the new WCT differentiation methods were found to be very accurate. The methods described could be automatically implemented by contemporary ECG interpretation software. The amplitude based and time-voltage area based WCT Formulas accurately predicted the vast majority of WCTs according to an institution's current clinical diagnosis practices. Although direct head-to-head comparisons were not undertaken, both WCT Formula methods compare favorably to the diagnostic performances ascribed to other ECG criteria or algorithms. Moreover, unlike established manual interpretation methods, the WCT Formulas are able to automatically provide accurate VT probability estimations for WCTs routinely encountered in clinical practice.

The fundamental purpose of every WCT differentiation criteria or algorithm is to help providers successfully differentiate WCTs. This invention provides examples of how modern-day ECG interpretation software could be used to help providers successfully differentiate VT and SWCT. This alternative approach to diagnosis has the natural advantage of automatically delivering precise estimations of VT probability to clinicians irrespective of their ECG interpretation abilities. In this manner, automated methods, like the amplitude based and time-voltage area based WCT Formulas, are particularly well-suited to help providers with less ECG interpretation experience and/or unrelated clinical expertise provide accurate and timely WCT diagnoses. The incorporation of the present invention into computerized ECG interpretation software systems will supplement current diagnostic strategies so to improve the quality of care provided to patients with WCT.

Furthermore, the WCT Formulas' principles could be applied by diagnostic ECG interpretation software to predict VT. As a result, the present invention is not limited to the WCT or PAC or PTVAC formulas. This new electrophysiological principle (degree of QRS complex change in amplitude or time-voltage area between the WCT and baseline ECG helps distinguish VT and SWCT) can be utilized by ECG interpretation software to render precise and accurate predictions of VT or SWCT.

Other embodiments of these similar WCT differentiation methods may be automatically implemented by computerized ECG interpretation (CEI) software. For example, another method which uses mathematically-synthesized signals and/or more sophisticated machine learning techniques would serve as an alternative means to apply the electrophysiological principles of the WCT differentiation method.

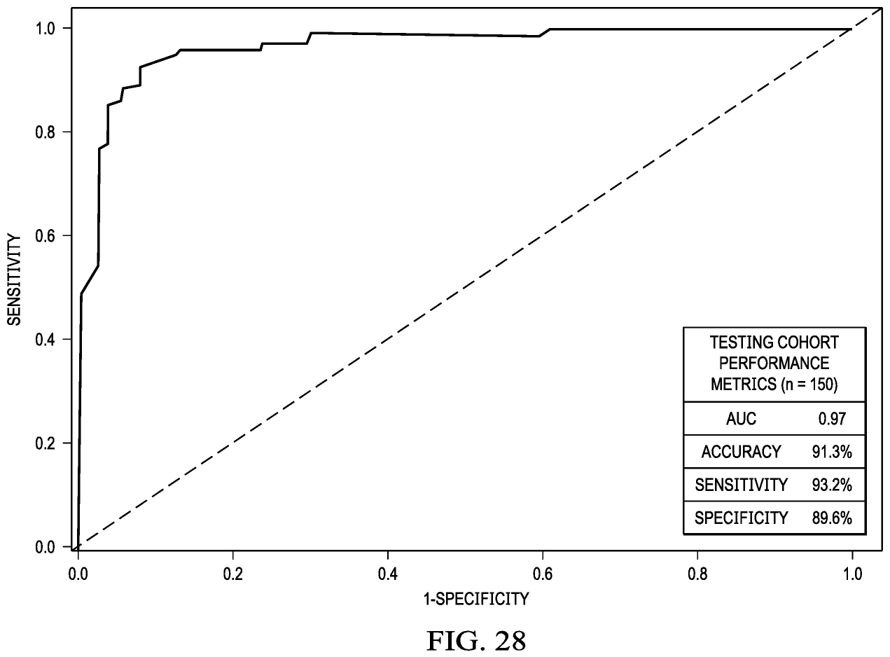

For example, in a two-part analysis, paired WCT and baseline ECGs were used to derive and test a Random Forests model (i.e. VCG-VT Model) comprised of standard computerized ECG measurements and novel computations formulated from mathematically-synthesized vectorcardiogram (VCG) signals. These mathematically-synthesized vectorcardiogram (VCG) signals are derived from the electrical signals acquired from the 12-lead ECG. A derivation cohort comprised of 450 WCT (199 VT, 251 SWCT) and baseline ECG pairs was used to train a VCG-VT Model comprised of WCT QRS duration (ms), X-lead percent QRS amplitude change (%), Y-lead percent QRS amplitude change (%), and Z-lead percent QRS amplitude change (%). VCG-VT Model implementation on the testing cohort of 150 WCT (73 VT, 77 SWCT) and baseline ECG pairs resulted in an overall AUC, accuracy, sensitivity, and specificity of 0.97 (CI 0.94-0.99), 91.3% (CI 85.6%-95.3%), 93.2% (CI 84.7%-97.7%), and 89.6% (CI 80.6%-95.4%), respectively as shown in FIG. 28.

Additionally, the WCT Formula's electrophysiological principles may be applied to a wide variety of ECG, EMG, VCG and/or mathematically-synthesized VCG analysis platforms beyond the diagnostic 12-lead ECG, including continuous ECG telemetry monitors, stress testing systems, extended monitoring devices (e.g., Holter monitors, etc.), smartphone-enabled ECG medical devices, cardioverter-defibrillator therapy devices, such as wearable cardioverter defibrillators (e.g., Zoll Life Vest), subcutaneous implantable cardioverter defibrillators (ICD) (e.g., Emblem S-ICD by Boston Scientific), intracardiac or transvenouspacemaker devices, automated external defibrillators (AED) (e.g., HeartStart OnSite AED by Phillips), and conventional automatic implantable cardioverter defibrillators (AICD). Measurements and calculations of EMG signals recorded from intracardiac (e.g. right ventricular AICD coil) and/or extracardiac electrodes (e.g. AICD generator housing) may also be used to established the degree (or percentage) of change in amplitude or time-voltage area between the WCT and baseline ventricular EMGs to help distinguish VT and SWCT. A similar process may be used to determine the source of individual wide complex beats (premature ventricular contraction or supraventricular wide complex beat or ventricular pacing). This discrimination process could be used to determine the need to deliver of device-related therapies, including anti-tachycardia pacing and defibrillator shocks.

Various embodiments of the present invention will now be described. These embodiments are merely examples and are not intended to limit the scope of the invention.

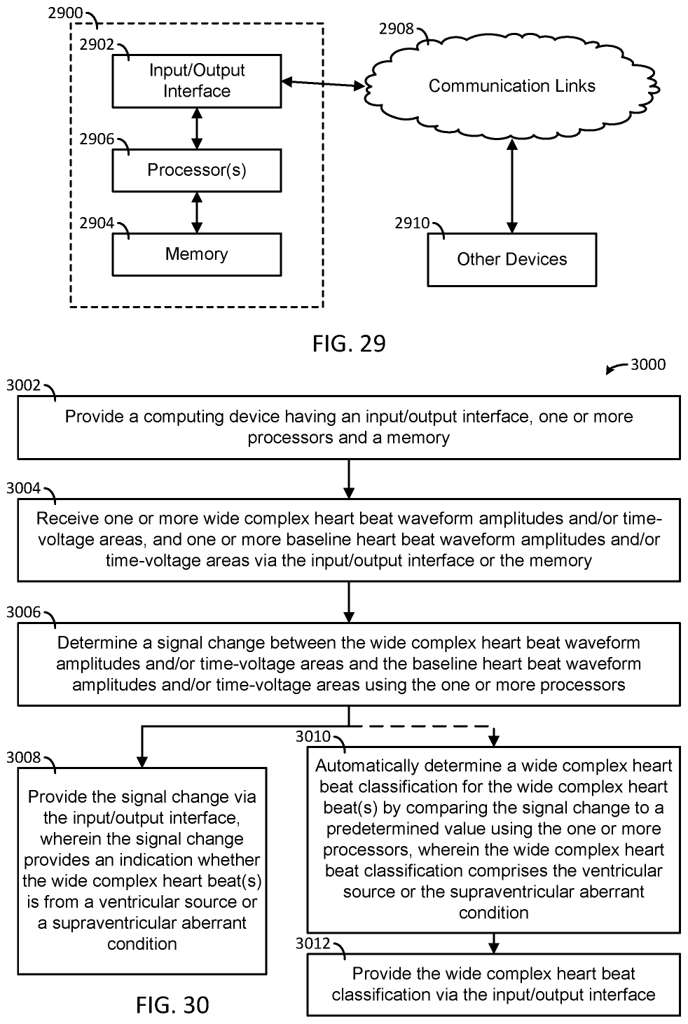

Now referring to FIG. 29, an apparatus 2900 for classifying a wide complex tachycardia (WCT) in accordance with the present invention is shown. The apparatus 2900 can be a server computer, a workstation computer, a laptop computer, a mobile communications device, a personal data assistant, a medical device or any other device capable of performing the functions described herein. The apparatus 2900 includes an input/output interface 2902, a memory 2904, and one or more processors 2906 communicably coupled to the input/output interface 2902 and the memory 2904. Note that the apparatus 2900 may include other components not specifically described herein. The memory 2904 can be local, remote or distributed. Likewise, the one or more processors 2906 can be local, remote or distributed. The input/output interface 2902 can be any mechanism for facilitating the input and/or output of information (e.g., web-based interface, touchscreen, keyboard, mouse, display, printer, etc.) Moreover, the input/output interface 2902 can be a remote device communicably coupled to the one or more processors 2906 via one or more communication links 2908 (e.g., network(s), cable(s), wireless, satellite, etc.). The one or more communication links 2908 can communicably couple the apparatus 2900 to other devices 2910 (e.g., databases, remote devices, hospitals, doctors, researchers, patients, etc.).

The one or more processors 2906 receive one or more wide complex heart beat waveform amplitudes and/or time-voltage areas, and one or more baseline heart beat waveform amplitudes and/or time-voltage areas via the input/output interface 2902 or the memory 2904, determine a signal change between the wide complex heart beat waveform amplitudes and/or time-voltage areas and the baseline heart beat waveform amplitudes and/or time-voltage areas, and provide the signal change via the input/output interface 2902, wherein the signal change provides an indication whether the wide complex heart beat(s) is from a ventricular source or a supraventricular source. In one embodiment, the delivery of a signal change, such as % VT probability, to clinicians provides an invaluable diagnostic tool that allows them to use their clinical judgement as how to manage the patient. In another embodiment, the one or more processors 2906 provide the signal change via the input/output interface 2902 by automatically determining a wide complex heart beat classification for the wide complex heart beat(s) by comparing the signal change to a predetermined value using the one or more processors, and providing the wide complex heart beat classification via the input/output interface 2902.

Referring now to FIG. 30, a flow chart of a computerized method 3000 of automatically classifying a wide complex heart beat(s) is shown. A computing device having an input/output interface, one or more processors and a memory is provided in block 3002. One or more wide complex heart beat waveform amplitudes and/or time-voltage areas, and one or more baseline heart beat waveform amplitudes and/or time-voltage areas are received via the input/output interface or the memory in block 3004. A signal change between the wide complex heart beat waveform amplitudes and/or time-voltage areas and the baseline heart beat waveform amplitudes and/or time-voltage areas is determined using the one or more processors in block 3006. The signal change is provided via the input/output interface in block 3008, wherein the signal change provides an indication whether the wide complex heart beat(s) is from a ventricular source or a supraventricular source. In another embodiment, a wide complex heart beat classification for the wide complex heart beat(s) is automatically determined by comparing the signal change to a predetermined value in block 3010, wherein the wide complex heart beat classification comprises the ventricular source or the supraventricular sourec. The wide complex heart beat classification is provided via the input/output interface in block 3012. The signal change can be concomitantly “weighted” with other predictors of VT, SWCT, ventricular pacing, supraventricular premature contraction or ventricular premature contraction. Moreover, the method can be implemented using a non-transitory computer readable medium that when executed causes the one or more processors to perform the method.

Now referring to FIGS. 29 and 30, other aspects of the present invention that are applicable to the apparatus 2900 and the method 3000 will now be described. In one aspect, the signal change further provides the indication whether the wide complex heart beat(s) is due to ventricular pacing or premature ventricular contractions. In another aspect, the wide complex heart beat(s) comprise a wide complex tachycardia (WCT), the ventricular source comprises a ventricular tachycardia (VT), and the supraventricular source comprises a supraventricular wide complex tachycardia (SWCT). In another aspect, the signal change comprises a VT probability, the wide complex heart beat classification comprises a VT whenever the VT probability is greater than or equal to the predetermined value, and the wide complex heart beat classification comprises a SWCT whenever the VT probability is less than the predetermined value. In another aspect, the one or more processors select the predetermined value from a range of 0% to 100%. In another aspect, the predetermined value comprises about 1%, 10%, 25%, 50%, 75%, 90% or 99%. In another aspect, the one or more processors provide the signal change by providing a “shock” recommendation signal, a “no shock” recommendation signal, or no signal. In another aspect, the wide complex heart beat waveform amplitudes and/or time-voltage areas and the baseline heart beat waveform amplitudes and/or time-voltage areas are obtained from an electrocardiogram (ECG) QRS signal, a ventricular electrogram (EMG) signal, a vectorcardiogram (VCG) and/or a mathematically-synthesized VCG signal. In another aspect, the wide complex heart beat waveform amplitudes and/or time-voltage areas comprise a plurality of measured amplitudes and/or time-voltage areas of a ECG QRS waveform, a EMG waveform, a VCG waveform and/or a mathematically-synthesized VCG waveformabove and below an isoelectric baseline; and the baseline heart beat waveform amplitudes and/or time-voltage areas comprise a plurality of measured amplitudes and/or time-voltage areas of a baseline ECG QRS waveform, a baseline EMG waveform, a baseline VCG waveform and/or a baseline mathematically-synthesized VCG waveform above and below the isoelectric baseline.

In another aspect, the one or more processors receive the one or more wide complex heart beat waveform amplitudes and/or time-voltage areas, and one or more baseline heart beat waveform amplitudes and/or time-voltage areas by: receiving a ECG QRS data, a EMG data, a VCG data and/or a mathematically-synthesized VCG data via the input/output interface or the memory; receiving a baseline ECG QRS data, a baseline EMG data, a baseline VCG data, and/or a baseline mathematically-synthesized VCG data via the input/output interface or the memory; determining the one or more waveform amplitudes and/or time-voltage areas from the ECG QRS data, the EMG data, the VCG data, and the mathematically-synthesized VCG data; and determining the one or more baseline waveform amplitudes and/or time-voltage areas from the baseline ECG QRS data, the baseline EMG data, the baseline VCG data, and/or the baseline mathematically-synthesized VCG data. In another aspect, the ECG QRS data, the EMG data, the VCG data and/or the mathematically-synthesized VCG data is generated or recorded before or after the baseline ECG QRS data, the baseline EMG data, the baseline VCG data, and/or the baseline mathematically-synthesized VCG data. In another aspect, the ECG QRS data, the EMG data, the VCG data, and/or the mathematically-synthesized VCG data is generated or recorded after the baseline ECG QRS data, the baseline EMG data, the baseline VCG data, and/or the baseline mathematically-synthesized VCG data and determining the signal change. In another aspect, the ECG QRS data, the EMG data, the VCG data, and/or the mathematically-synthesized VCG data and the baseline ECG QRS data, the baseline EMG data, the baseline VCG data, and/or the baseline mathematically-synthesized VCG data are generated or recorded using one or more sensors or devices. In another aspect, the one or more sensors or devices comprise a 12-lead ECG device, a continuous ECG telemetry monitor, a stress testing system, an extended monitoring device, a smartphone-enabled ECG medical device, a cardioverter-defibrillator therapy device, a subcutaneous implantable cardioverter defibrillators (S-ICD), a pacemaker, an automated external defibrillators (AED), or an automatic implantable cardioverter defibrillator (AICD). In another aspect, the input/output interface, the memory and the one or more processors are integrated into the one or more sensors or devices; or the one or more sensors or devices are integrated into a computing device comprising the input/output interface, the memory and the one or more processors. In another aspect, the one or more processors determine the signal change between the wide complex heart beat waveform amplitudes and/or time-voltage areas and the baseline heart beat waveform amplitudes and/or time-voltage areas by: receiving a wide complex heart beat waveform duration via the input/output interface or the memory; determining a percent amplitude change (PAC) based on the wide complex heart beat waveform amplitudes and the baseline heart beat waveform amplitudes, and/or a percent time-voltage area change (PTVAC) based on the wide complex heart beat waveform time-voltage areas and the baseline heart beat waveform time-voltage areas; determining a classification probability based on the wide complex heart beat waveform duration, and the PAC and/or the PTVAC; and wherein the signal change comprises the classification probability, and the classification probability comprises a VT probability, a SWCT probability, or a ventricular pacing probability. In another aspect, determining the classification probability is further determined based one or more additional classification predictors. In another aspect, the PAC comprises a frontal PAC and a horizontal PAC, and the PTVAC comprises a frontal PTVAC and a horizontal PTVAC.

In another aspect, the one or more processors determine the signal change between the wide complex heart beat waveform amplitudes and/or time-voltage areas and the baseline heart beat waveform amplitudes and/or time-voltage areas by: receiving a WCT QRS duration via the input/output interface or the memory; the one or more wide complex heart beat waveform amplitudes and/or time-voltage areas comprise one or more frontal plane WCT positive waveform amplitudes and/or time-voltage areas, one or more horizontal plane WCT positive waveform amplitudes and/or time-voltage areas, one or more frontal plane WCT negative waveform amplitudes and/or time-voltage areas, and one or more horizontal plane WCT negative waveform amplitudes and/or time-voltage areas; the one or more the baseline heart beat waveform amplitudes and/or time-voltage areas comprise one or more frontal plane baseline positive waveform amplitudes and/or time-voltage areas, one or more horizontal plane baseline positive waveform amplitudes and/or time-voltage areas, one or more frontal plane baseline negative waveform amplitudes and/or time-voltage areas, and one or more horizontal baseline negative waveform amplitudes and/or time-voltage areas; determining (1) a frontal percent amplitude change (PAC) based on the one or more frontal plane WCT positive waveform amplitudes, one or more frontal plane WCT negative waveform amplitudes, one or more frontal plane baseline positive waveform amplitudes, and one or more frontal plane baseline negative waveform amplitudes, and/or (2) a frontal percent time-voltage area (PTVAC) based on the one or more frontal plane WCT positive waveform time-voltage areas, one or more frontal plane WCT negative waveform time-voltage areas, one or more frontal plane baseline positive waveform time-voltage areas, and one or more frontal plane baseline negative waveform time-voltage areas; determining (1) a horizontal PAC based on the one or more horizontal plane WCT positive waveform amplitudes, one or more horizontal plane WCT negative waveform amplitudes, one or more horizontal plane baseline positive waveform amplitudes, and one or more horizontal baseline negative waveform amplitudes, and/or (2) a horizontal PTVAC based on the one or more horizontal plane WCT positive waveform time-voltage areas, one or more horizontal plane WCT negative waveform time-voltage areas, one or more horizontal plane baseline positive waveform time-voltage areas, and one or more horizontal baseline negative waveform time-voltage areas; determining a VT probability using a statistical or machine learning process based on the WCT QRS duration and (1) the frontal PAC and the horizontal PAC, and/or (2) the frontal PTVAC and the horizontal PTVAC; and wherein the signal change comprises the VT probability. In another aspect, the statistical or machine learning process comprises a linear regression algorithm, a logistic regression model, a linear discriminate analysis algorithm, a Naive Bayes algorithm, a computational model using artificial neural networks, a computational model based on classification or regression trees, a k-nearest neighbors based model, a support vector machine based model, a boosting algorithm, or an ensemble machine learning algorithm.

In another aspect, the frontal PAC is determined by

$$Frontal PAC ( \% ) = ( \frac{Frontal AAC}{Frontal BA} ) \times 100 ,$$

where: Frontal AAC=TAC_{aVR}+TAC_{aVL}+TAC_{aVF}, Frontal BA=TBA_{aVR}+TBA_{aVL}+TBA_{aVF}, TAC_{LeadX}=APC_{LeadX}+ANC_{LeadX}, TBA_{Baseline:LeadX}=(−)Amplitude_{Baseline:LeadX}+(+)Amplitude_{Baseline:LeadX}, APC_{LeadX}=|(+)Amplitude_{WCT:LeadX}−(+)AMplitude_{Baseline:LeadX}|, ANC_{LeadX}=|(−)Amplitude_{WCT:LeadX}−(−)Amplitude_{Baseline:LeadX}|, LeadX denotes V1, V4, V6 (horizontal plane) or aVL, aVR, aVF (frontal plane); the horizontal PAC is determined by

$$Horizontal PAC ( \% ) = ( \frac{Horizontal AAC}{Horizontal BA} ) \times 100 ,$$

where: Horizontal AAC=TAC_{V1}+TAC_{V4}+TAC_{V6}, Horizontal BA=TBA_{V1}+TBA_{V4}+TBA_{V6}; and the VT probability (P_{VT}) is determined by:

$${P}_{V T} = \frac{{e}^{( a + b \times {WCT}_{duration} + c \times {PAC}_{frontal} + d \times {PAC}_{horizontal} )}}{1 + {e}^{( a + b \times {WCT}_{duration} + c \times {PAC}_{frontal} + d \times {PAC}_{horizontal} )}} ,$$

where a, b, c and d are constants. In another aspect, the frontal PTVAC is determined by

$$Frontal PTVAC ( \% ) = ( \frac{Frontal ATVAC}{Frontal BTVA} ) \times 100 ,$$

where: Frontal ATVAC=TTVAC_{aVR}+TTVAC_{aVL}+TTVAC_{aVF}, Frontal BTVA=TBTVA_{aVR}+TBTVA_{aVL}+TBTVA_{aVF}, TTVAC_{LeadX}=TVAPC_{LeadX}+TVANC_{LeadX}, TBTVA_{Baseline:LeadX}=(−)TimeVoltageArea_{Baseline:LeadX}+(+)TimeVoltageArea_{Baseline:LeadX}, TVAPC_{leadX}=|(+)TimeVoltageArea_{WCT:LeadX}−(+)TimeVoltageArea_{Baseline:LeadX}|, TVANC_{leadX}=|(−)TimeVoltageArea_{WCT:LeadX}−(−)TimeVoltageArea_{Baseline:LeadX}|, LeadX denotes V1, V4, V6 (horizontal plane) or aVL, aVR, aVF (frontal plane); the horizontal PTVAC is determined by

$$Horizontal PTVAC ( \% ) = ( \frac{Horizontal ATVAC}{Horizontal BTVA} ) \times 100 ,$$

where: Horizontal ATVAC=TTVAC_{V1}+TTVAC_{V4}+TTVAC_{V6},

Horizontal BTVA=TBTVA_{V1}+TBTVA_{V4}+TBTVA_{V6}; and the VT probability (P_{VT}) is determined by:

$${P}_{V T} = \frac{{e}^{( a + b \times {WCT}_{duration} + c \times {PTVAC}_{frontal} + d \times {PTVAC}_{horizontal} )}}{1 + {e}^{( a + b \times {WCT}_{duration} + c \times {PTVAC}_{frontal} + d \times {PTVAC}_{horizontal} )}} ,$$

where: a, b, c and d are constants.

In another aspect, the input/output interface comprises a remote device, and the remote device is communicably coupled to the one or more processors via one or more networks. In another aspect, the one or more processors provide a recommendation to select or exclude a therapy, medication, diagnostic testing or referral for a patient based on the signal change. In another aspect, apparatus comprises a server computer, a workstation computer, a laptop computer, a mobile communications device, a personal data assistant, or a medical device.

VT Prediction Model Embodiments

The following types of embodiments of the present invention provide a WCT differentiation method that is based in whole or in part on a VT prediction model. This simplified means of WCT differentiation operates solely on computerized measurements routinely displayed on 12-lead ECG paper recordings. Through an exclusive examination of computerized ECG measurements provided by paired WCT and baseline ECGs, a logistic regression prediction model (i.e., VT Prediction Model) was constructed that accurately distinguishes VT and SWCT.

In a two-part study, a logistic regression model (i.e VT Prediction Model) composed of computerized measurements and calculations derived from paired WCT and baseline ECGs was developed and validated. In Part 1, a logistic regression model (i.e. VT Prediction Model) was derived from 601 paired WCT and subsequent baseline ECGs. In Part 2, The VT Prediction Model's performance was tested using a separate validation cohort of 241 paired WCT and baseline ECGs. Patient data acquisition and analysis was approved by the Mayo Clinic Institutional Review Board.

Electrocardiograms were standard, 12-lead recordings (paper speed: 25 mm/s, voltage calibration: 10 mm/mV) identified within our institution's centralized MUSE ECG data archives (GE Healthcare; Milwaukee, Wis.). Wide complex tachycardias were defined as ECGs fulfilling WCT criteria (QRS duration≥120 ms; heart rate ≥100 bpm) plus a formal ECG laboratory interpretation of (1) “ventricular tachycardia,” (2) “supraventricular tachycardia,” or (3) “wide complex tachycardia.” Baseline ECGs were defined as being either the first subsequent ECG (i.e., for the derivation cohort) or most proximate ECG (i.e., for the validation cohort) not meeting WCT criteria. Only WCTs with a paired baseline ECG and definite clinical diagnosis established by the patient's overseeing physician were analyzed. Polymorphic VTs and SWCTs with varying atrioventricular conduction (e.g. atrial fibrillation) were not evaluated. Abbreviated WCTs (e.g. non-sustained VT) occurring within a dominant baseline ECG rhythm (e.g. normal sinus rhythm) were excluded. Paired ECGs demonstrating erroneous computerized ECG measurements due to ECG artifact were excluded.

The derivation cohort consisted of 601 paired WCT (273 VT, 328 SWCT) and baseline ECGs from 421 patients presenting to Mayo Clinic Rochester and/or Mayo Clinic Health System of South Eastern Minnesota (September 2011 to November 2016). Clinical and electrocardiographic data of the derivation cohort was previously examined in a separate analysis (13,14). The validation cohort comprised 241 WCT (97 VT, 144 SWCT) and baseline ECG pairs from 177 patients presenting to the entire Mayo Clinic enterprise (January 2018 to December 2018)—including three tertiary medical centers (Rochester, Minn.; Jacksonville, Fla.; and Phoenix/Scottsdale, Ariz.) and other affliated patient care locations (e.g., surrounding community hospitals and outpatient clinics).

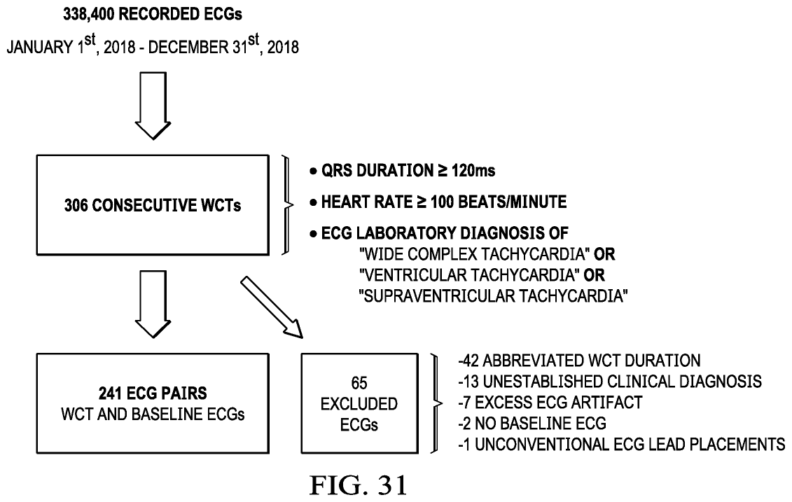

FIG. 31 shows a flow diagram of validation cohort selection. Sixty-five out of 306 consecutive WCTs were excluded. Forty-two of those excluded were abbreviated WCTs occurring within a dominant baseline heart rhythm. Thirteen WCTs did not have an established clinical diagnosis. Seven ECG pairs were disqualified due to due excessive ventricular assist device artifact. Two WCTs did not have a corresponding baseline ECG. One WCT was recorded using erroneous ECG lead placements (i.e. limb lead reversal).

Clinical diagnoses (VT or SWCT) were established by the patient's overseeing physician. Physicians responsible for clinical diagnoses were classified according to their level of expertise: (1) heart rhythm cardiologist, (2) non-heart rhythm cardiologist and (3) non-cardiologist. The “most expert” overseeing physician (heart rhythm cardiologist >non-heart rhythm cardiologist >non-cardiologist) determined the patient's final clinical diagnosis. All overseeing physicians had access to interpretive diagnoses provided by the ECG laboratory.

Formal ECG laboratory interpration was provided by supervising physician interpreters according to the established site-specific practices of various patient care locations across the Mayo Clinic enterprise. Six heart rhythm cardiologists and 12 non-heart rhythm cardiologists were responsible for ECG interpretations of WCTs comprising the derivation cohort. Eleven heart rhythm cardiologists and 18 non-heart rhythm cardiologists were responsible for ECG interpretations of WCTs comprising the validation cohort.

The ECG interpretation strategy(s) utilized to differentiate WCTs was determined by the supervising interpreter. Supervising interpreters had access to patients' electronic medical record and archived ECGs at the time of ECG interpretation. Interpretive diagnoses were semi-qualitatively re-categorized according to diagnostic certainty: (1) definite VT, (2) probable VT, (3) definite SWCT, (4) probable SWCT and (5) undifferentiated. The time separation between paired WCT and baseline ECGs was recorded.

Clinical data including patient age, gender, prior myocardial infarction, structural heart disease, baseline bundle branch block (BBB), implanted automatic implantable cardioverter-defibrillator (AICD), and ongoing Vaughan-Williams Class I and III antiarrhythmic drug use were obtained from the electronic medical record. The completion of an electrophysiology procedure supplementing the patient's clinical diagnosis was recorded.

The VT Prediction Model constituents include data that is obtained from standard computerized ECG measurements, namely WCT QRS duration (ms), QRS duration change (ms), QRS axis change (°), and T axis change (°). QRS duration change is the absolute difference in QRS duration (ms) measurements between paired WCT and baseline ECGs was calculated. QRS axis change is the absolute difference in frontal plane QRS axis) (° between paired WCT and baseline ECGs was calculated. The magnitude of QRS axis change ranged from 0° (i.e. no axis shift) to 180° (i.e. complete axis shift to the straight angle opposite). T axis change is the absolute difference in frontal plane T axis (°) between paired WCT and baseline ECGs was calculated. The magnitude of T axis change ranged from 0° (i.e. no axis shift) to 180° (i.e. complete axis shift to the straight angle opposite). Before performing an axis change calculations, frontal plane axes were converted into a different mapping rubric for directionality which utilizes 1°-360° instead of ECG paper recordings description of axis (i.e. negative axis numbers (i.e. −1° through-90°) were changed to positive axis numbers (i.e. 359° through 270°), respectively). Although absolute values were used in the analysis, non-absolute values can be used for various prediction models (e.g. artificial neural networks).

The VT Prediction Model integrates computerized ECG measurements and basic mathematical computations derived from paired WCT and baseline ECGs to generate an estimation of VT probability (0.000%-99.999%). The logistic regression structure of the VT Prediction Model is outlined below:

$${X}_{β} = {β}_{0} + {β}_{1} {X}_{1} + {β}_{2} {X}_{2} + {β}_{3} {X}_{3} = Ln ( \frac{{P}_{V T}}{1 - {P}_{V T}} )$$

$${X}_{β} = - 10.69 + ( 0.0211 ) ( QRS axis change ) + ( 0.0124 ) ( T axis change ) + ( 0.0532 ) ( WCT QRS duration ) + ( 0.00123 ) ( QRS duration change )$$

$${P}_{V T} = \frac{{e}^{( - 10.69 + 0.0211 \times ( QRS axis change ) + 0.0124 \times ( T axis change ) + 0.0532 \times ( WCT QRS duration ) + 0.00123 \times ( QRS duration change ) )}}{1 + {e}^{( - 10.69 + 0.0211 \times ( QRS axis change ) + 0.0124 \times ( T axis change ) + 0.0532 \times ( WCT QRS duration ) + 0.00123 \times ( QRS duration change ) )}}$$

Independent explanatory variables (Xₓ) include: WCT QRS duration (ms), QRS duration change (ms), QRS axis change (°), and T axis change (°). Beta coefficients (βₓ) were assigned to each VT predictor (Xₓ) according to their influence on the binary outcome (VT or SWCT). The “constant” term (B₀) is the y-intercept of the least squares regression line. Estimated VT probability (P) and the weighted sum predictor (X_{β}) are derived from integrated VT predictor (Xₓ) values.

Accordingly, the VT probability (P_{VT}) is determined by:

$${P}_{V T} = \frac{{e}^{( a + b \times ( R axis change ) + c \times ( T axis change ) + d \times ( WCT QRS duration ) + g \times ( QRS duration change ) )}}{\begin{matrix}1 + \\ {e}^{( a + b \times ( R axis change ) + c \times ( T axis change ) + d \times ( WCT QRS duration ) + g \times ( QRS duration change ) )}\end{matrix}} ,$$

where: a, b, c, d and g are constants.

The changes in the R wave axis, T wave axis, and QRS duration can be absolute values or non-absolute values as shown below. These computationally engineering inputs can be fed into the VT Prediction Modes.

Non-absolute change in R wave axis→values −179 to 180°:

$$( WCT R axis - Baseline R axis ) + If \{ \begin{matrix}180 \leq WCT R axis - Baseline R axis < 360 ⇒ 0 - 360 \\ else ⇒ 0\end{matrix} \} + If \{ \begin{matrix}- 360 < WCT R axis - Baseline R axis \leq - 180 ⇒ 360 \\ else ⇒ 0\end{matrix} \}$$

Absolute change in R wave axis→values 0 to 180°:

$$\vert \begin{matrix}( WCT R axis - Baseline R axis ) + \\ If \{ \begin{matrix}180 \leq WCT R axis - Baseline R axis < 360 ⇒ 0 - 360 \\ else ⇒ 0\end{matrix} \} + \\ If \{ \begin{matrix}- 360 < WCT R axis - Baseline R axis \leq - 180 ⇒ 360 \\ else ⇒ 0\end{matrix} \}\end{matrix} \vert$$

Non-absolute change in T wave axis→values −179 to 180°:

$$( WCT T axis - Baseline T axis ) + If \{ \begin{matrix}180 \leq WCT T axis - Baseline T axis < 360 ⇒ 0 - 360 \\ else ⇒ 0\end{matrix} \} + If \{ \begin{matrix}- 360 < WCT T axis - Baseline T axis \leq - 180 ⇒ 360 \\ else ⇒ 0\end{matrix} \}$$

Absolute change in T wave axis→values 0 to 180°:

$$\vert \begin{matrix}( WCT T axis - Baseline T axis ) + \\ If \{ \begin{matrix}180 \leq WCT T axis - Baseline T axis < 360 ⇒ 0 - 360 \\ else ⇒ 0\end{matrix} \} + \\ If \{ \begin{matrix}- 360 < WCT T axis - Baseline T axis \leq - 180 ⇒ 360 \\ else ⇒ 0\end{matrix} \}\end{matrix} \vert$$

Non-absolute change in QRS duration→positive and negative values:

(WCT QRS duration−Baseline QRS duration)

Absolute change in QRS duration→positive values only:

|WCT QRS duration−Baseline QRS duration|

The VT Prediction Model operates on readily available measurements provided by contemporary ECG interpretation software including QRS duration (ms), corrected QT interval duration (ms), QRS axis (°) and T axis (°). In this manner, newly formulated (e.g., T axis change) and/or well-established (e.g., WCT QRS duration) VT predictors may be incorporated into automated prediction models (e.g., logistic regression) that do not depend on clinicians' manual application of conventional ECG criteria. Furthermore, this approach enables the use of sophisticated “machine learning” methods (e.g., artificial neural networks or support vector machines) more apt to decipher non-linear and non-parametric data relationships. In this manner, the same basic ECG measurements used by the VT Prediction Model can be used by successive model iterations better able to distinguish VT and SWCT.

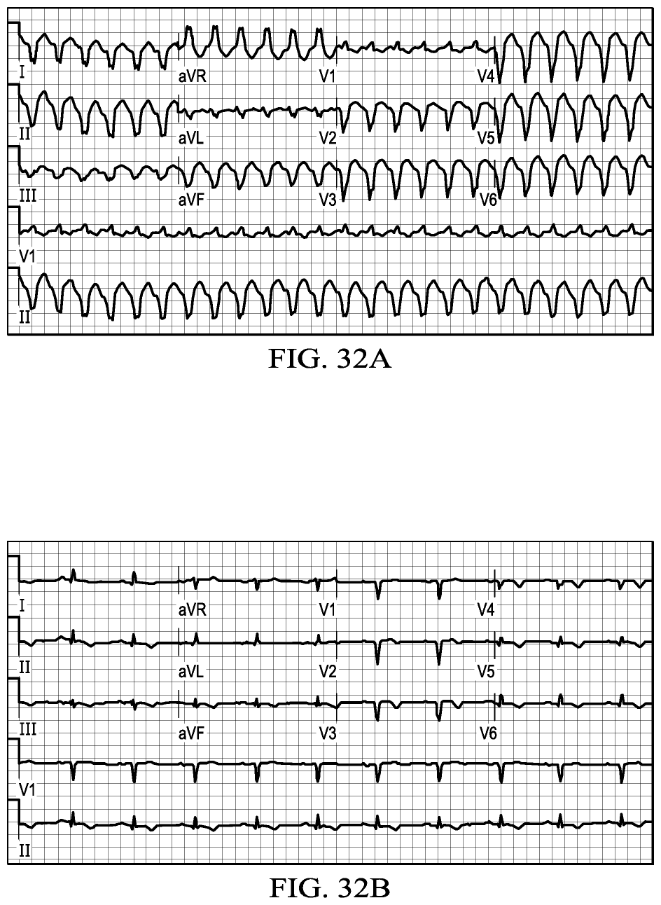

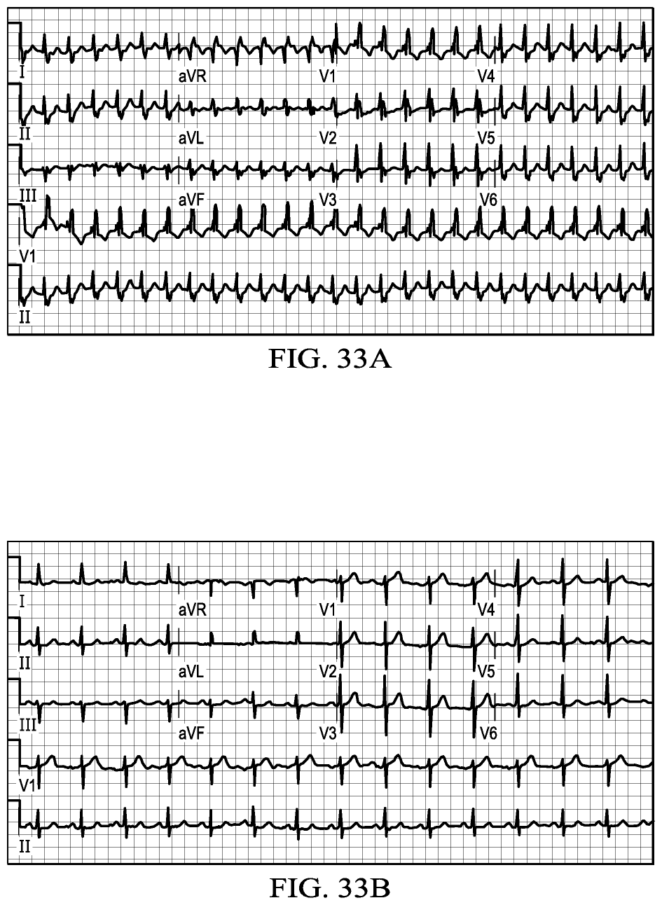

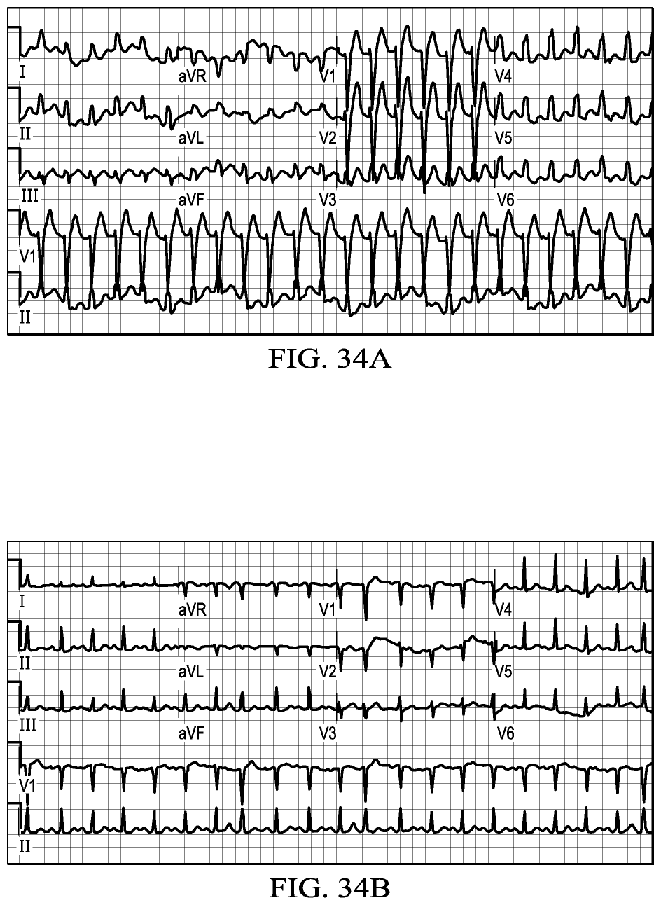

The VT Prediction Model assigns unambiguous VT probabilities (0.000%-99.999%) using independent VT predictors simultaneously “weighed” according to their influence on the binary outcome (VT vs. SWCT). Given each VT predictor's direct relationship with VT probability, the VT Prediction Model estimates higher VT probability for ECG pairs demonstrating greater WCT QRS duration, QRS duration change, QRS axis change and/or T axis change. FIGS. 32A-32B depict examples of paired VT (FIG. 32A) and baseline (FIG. 32B) ECGs assigned high VT probability (99.0006%) by the VT Prediction Model (WCT QRS duration=182 ms; QRS duration change=48 ms; QRS axis change=134°; T axis change=93°). In an opposite manner, the VT Prediction Model estimates lower VT probability for ECG pairs with smaller WCT QRS duration, QRS duration change, QRS axis change and/or T axis change. FIGS. 33A-33B depict examples of paired SWCT (FIG. 33A) and baseline (FIG. 33B) ECGs assigned low VT probability (4.3609%) by the VT Prediction Model (WCT QRS duration=130 ms; QRS duration change=46 ms; QRS axis change=1°; T axis change=8°). FIGS. 34A-34B depict examples of paired SWCT (FIG. 34A) and baseline (FIG. 34B) ECGs assigned low VT probability (6.3613%) by the VT Prediction Model (WCT QRS duration=120 ms; QRS duration change=36 ms; QRS axis change=48°; change T axis change=) 13°.

Categorical variables were compared using Chi-square tests. Comparisons of continuous variables were completed using Wilcoxon rank-sum tests. Stepwise logistic regression of explanatory variables—baseline QRS duration, baseline QTc duration, WCT QRS duration, QRS duration change, QRS axis change and T axis change—was used to fit the VT Prediction Model. Only independent explanatory variables were incorporated into the VT Prediction Model. Histograms and a receiver operator characteristic curves were used to summarize the VT Prediction Model's overall diagnostic performance. The accuracy, sensitivity, specificity, positive predictive value, negative predictive value, and positive/negative likelihood ratios of various VT probability partitions were evaluated according to their agreement with the overseeing physician's clinical diagnosis. Kappa (κ) statistics were used to assess agreement between the VT Prediction Model's 50% VT probability partition (VT=>50%; SWCT<50%), clinical diagnosis, and ECG laboratory interpretation. Agreement was characterized as “almost perfect” (κ=0.81-1.00), “substantial” (κ=0.61-0.80), “moderate” (κ=0.41-0.60), “fair” (κ=0.20-0.40), “slight” (κ=0.00-0.20), and “poor” (κ<0.00)(13). Statistical analyses were completed using SAS version 9.4 (SAS Institute, Cary, N.C.).

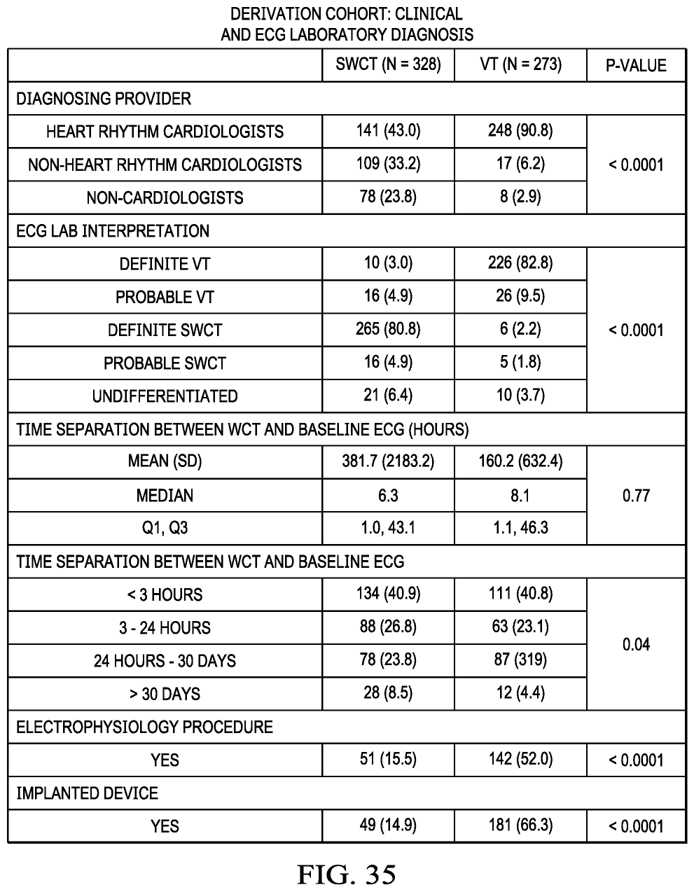

Clinical diagnosis and ECG laboratory interpretation data is summarized in FIG. 35. The numbers in parentheses are percent (%) of n or standard deviation. The majority (85.7%) of clinical diagnoses were established by heart rhythm or non-heart rhythm cardiologists. Most (94.8%) WCTs were assigned definitive or probable interpretive diagnoses by the ECG laboratory. Just over half (50.6%) of WCTs were derived from patients who underwent an electrophysiology procedure and/or possessed an implanted intracardiac device.

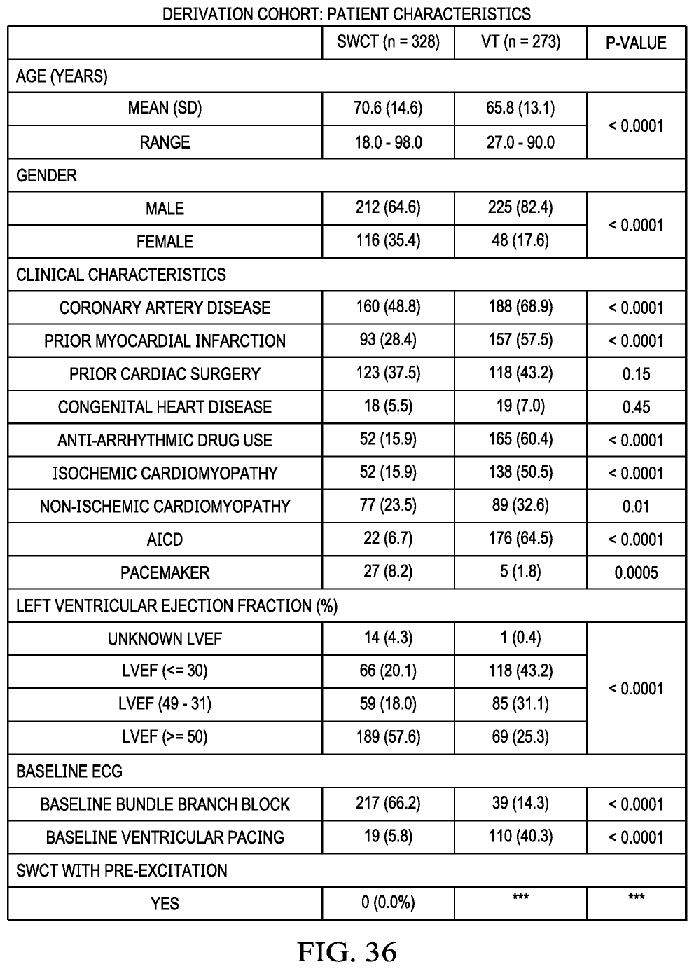

Patient characteristics of VT and SWCT groups are summarized in FIG. 36. The numbers in parentheses are percent (%) of n or standard deviation (SD). AICD is automatice implantable cardioverter-defibrillator. LVEF is left ventricular ejection fraction. The VT group included more ECG pairs from patients with coronary artery disease, prior MI, ongoing antiarrhythmic drug use, ischemic cardiomyopathy, non-ischemic cardiomyopathy and implanted AICD. The VT group comprised more ECG pairs from patients with reduced LVEF (<50%), whereas the SWCT group included more ECG pairs from patients with preserved LVEF (>=50%). Baseline ECGs with ventricular pacing were more common in the VT group, while baseline BBB was more common in the SWCT group. No SWCTs demonstrated atrioventricular pre-excitation.

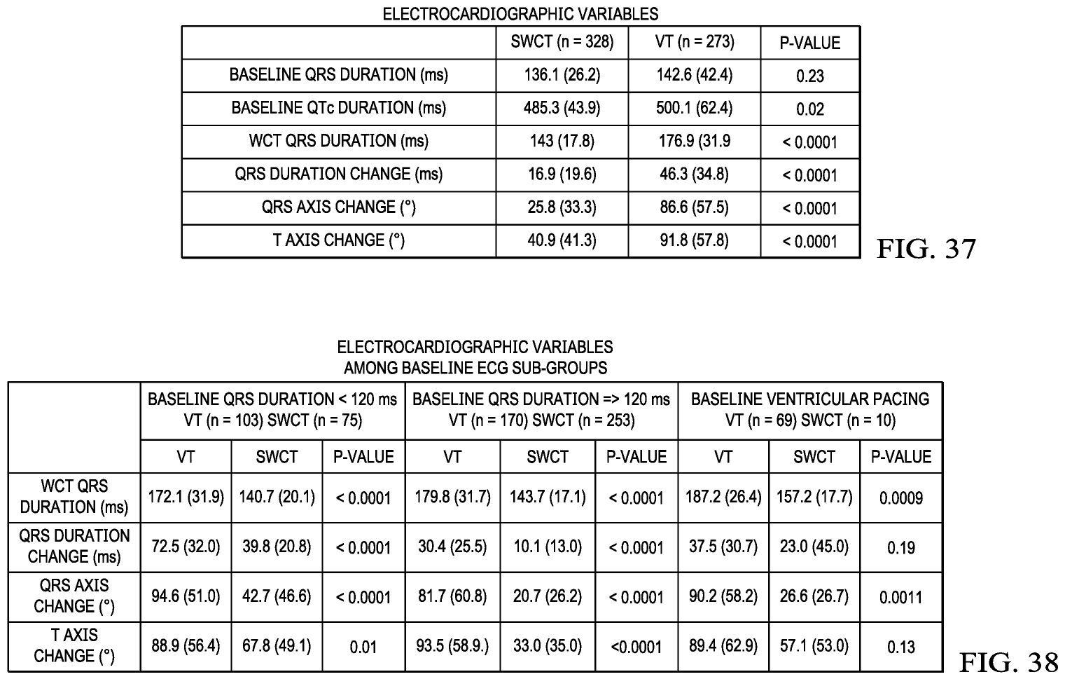

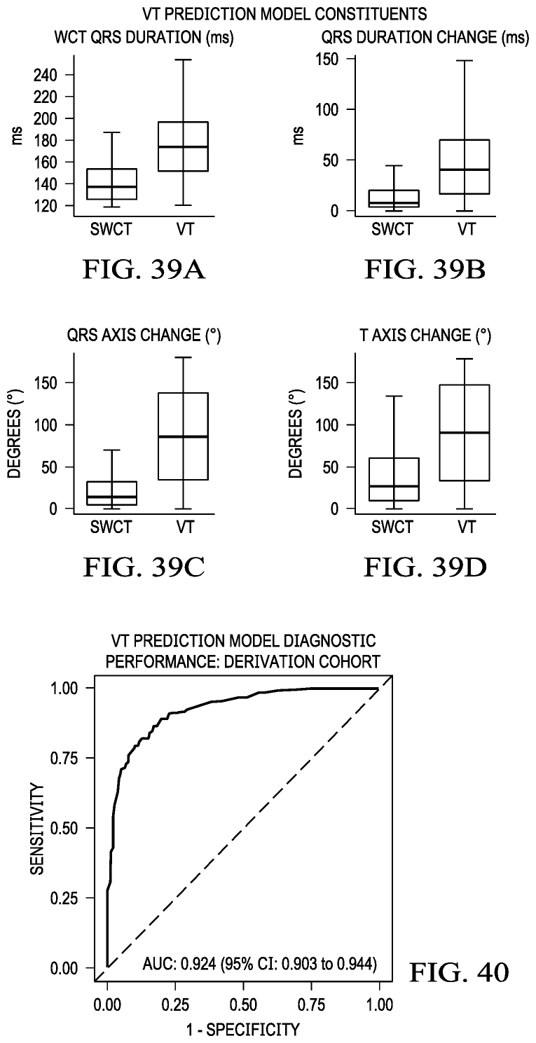

Paired ECGs in the VT group expressed greater baseline ECG QTc duration, WCT QRS duration, QRS duration change, QRS axis change and T axis change (FIG. 37). The numbers in parentheses are percent (%) of n or standard deviation (SD). The VT group also demonstrated greater WCT QRS duration, QRS duration change, QRS axis change and T axis change among baseline ECG subgroups with or without QRS duration prolongation (=>120 ms) (FIG. 38). The numbers in parentheses are standard deviation. Among baseline ECGs with ventricular pacing, the VT group demonstrated greater WCT QRS duration and QRS axis change. A summary of the median and proportional distribution of the VT Prediction Model's constituents is shown in FIGS. 39A-39D. The box-plots demonstrate the median and proportional distribution of WCT QRS duration (ms) (FIG. 39A), WCT QRS duration change (ms) (FIG. 39B), QRS axis change (°) (FIG. 39C) and T axis change (°) (FIG. 39D).

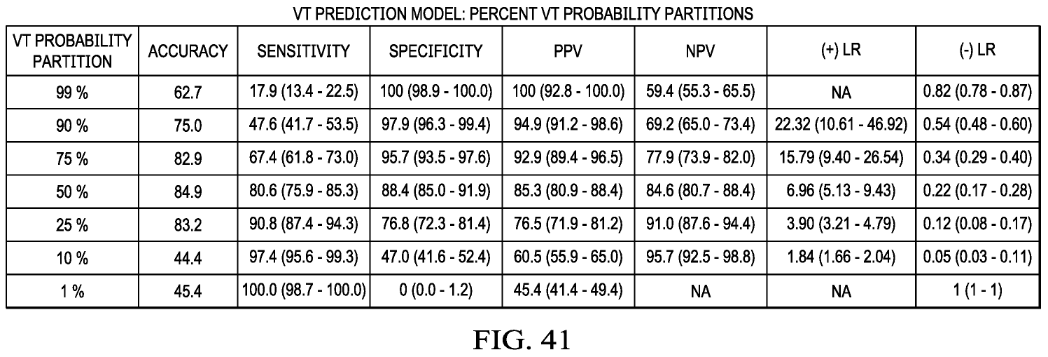

The VT Prediction Model composed of WCT QRS duration (ms) (p<0.0001), QRS duration change (ms) (p<0.0001), QRS axis change (°) (p<0.0001) and T axis change (°) (p<0.0001) demonstrated favorable WCT differentiation (AUC 0.924; CI 0.903-0.944) for the derivation cohort. FIG. 40 is a plot showing the receiver operating characteristic curve for the VT Prediction Model (AUC of 0.924; CI 0.903-0.944). Overall, the VT Prediction Model yielded an accuracy, sensitivity and specificity of 84.9%, 80.6% and 88.4%, respectively. The VT Prediction Model's diagnostic performance according to various VT probability partitions is summarized in FIG. 41. FIG. 41 is a table showin the percent VT probability using the VT Prediction Model. Displayed numbers represent percentage (%) values according to VT diagnoses. Numbers in parentheses represent 95% confidence interval range. LR=Likelihood Ratio. NA=Not Applicable. NPV=Negative Predictive Value. PPV=Positive Predictive Value. (−)=Negative. (+)=Positive. When using a 50% VT probability partition (VT>=50%; SWCT<50%), overall accuracy did not differ among patients with (83.4%) or without (85.5%) an accompanying electrophysiology procedure (p=0.50).

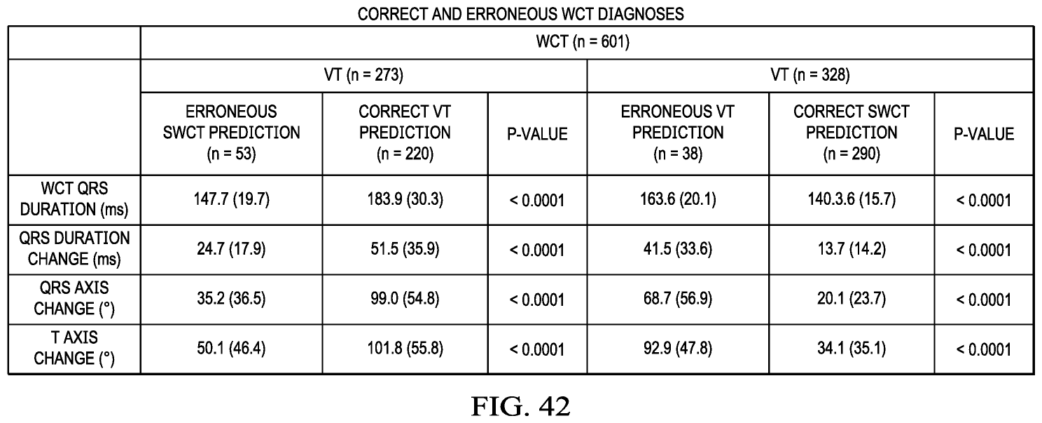

The electrocardiographic characteristics of correct and incorrect diagnoses established by the VT Prediction Model for the derivation cohort are summarized in FIG. 42. Displayed numbers represent mean values. Numbers in parentheses are standard deviation. The erroneous VT prediction group comprises clinical SWCTs assigned high VT probability (>=50%). The erroneous SWCT prediction group comprises clinical VTs assigned a low VT probability (<50%). Fifty-three out of 278 (19.1%) clinical VTs were erroneously categorized as SWCT. When compared to correctly identified VTs, erroneous classifications of clinical VT as SWCT exhibited shorter WCT QRS duration and limited changes in QRS duration, QRS axis and T axis between paired baseline and WCT ECGs. Thirty-eight out of 323 (11.8%) clinical SWCTs were erroneously categorized as VT. When compared to correctly identified SWCTs, erroneous classifications of clinical SWCT as VT demonstrated more prolonged WCT QRS duration and greater changes in QRS duration, QRS axis and T axis between paired baseline and WCT ECGs.

For VT diagnoses, the VT Prediction Model yielded moderate to substantial agreement with ECG laboratory interpretation (κ=0.64, CI 0.57-0.70) and clinical diagnosis (κ=0.69, CI 0.64-0.75). For SWCT diagnoses, the VT Prediction Model yielded moderate to substantial agreement with ECG laboratory interpretation (κ=0.64, CI 0.57-0.70) and clinical diagnosis (κ=0.69, CI 0.64-0.75).

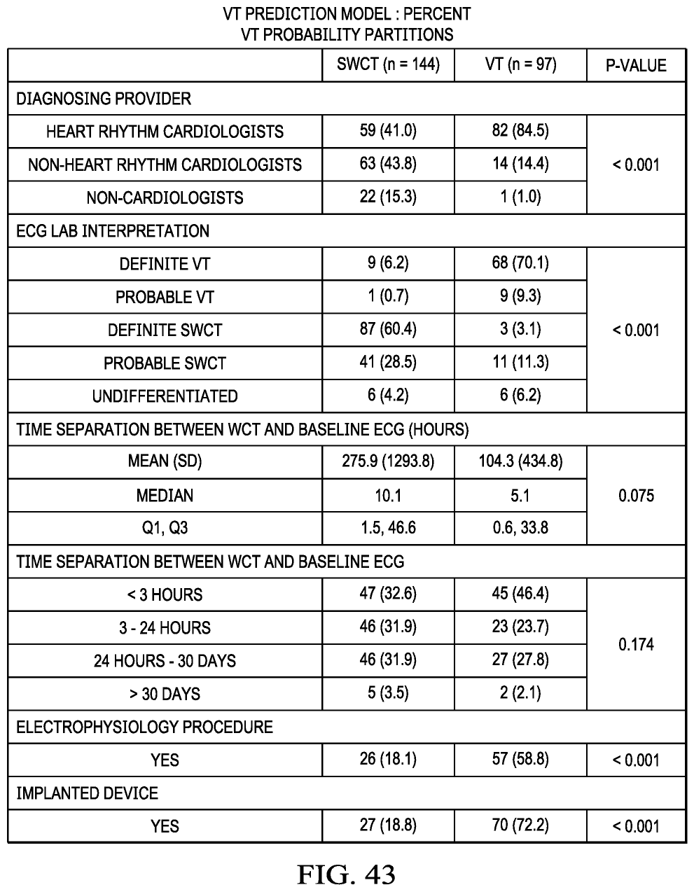

Clinical diagnosis and ECG laboratory interpretation data of the validation cohort is summarized in FIG. 43. The vast majority (90.5%) of clinical diagnoses were established by heart rhythm or non-heart rhythm cardiologists. Most (95.0%) WCTs were assigned definitive or probable interpretive diagnoses by the ECG laboratory. Over half (55.6%) of WCTs were derived from patients who underwent an electrophysiology procedure and/or possessed an implanted intracardiac device.

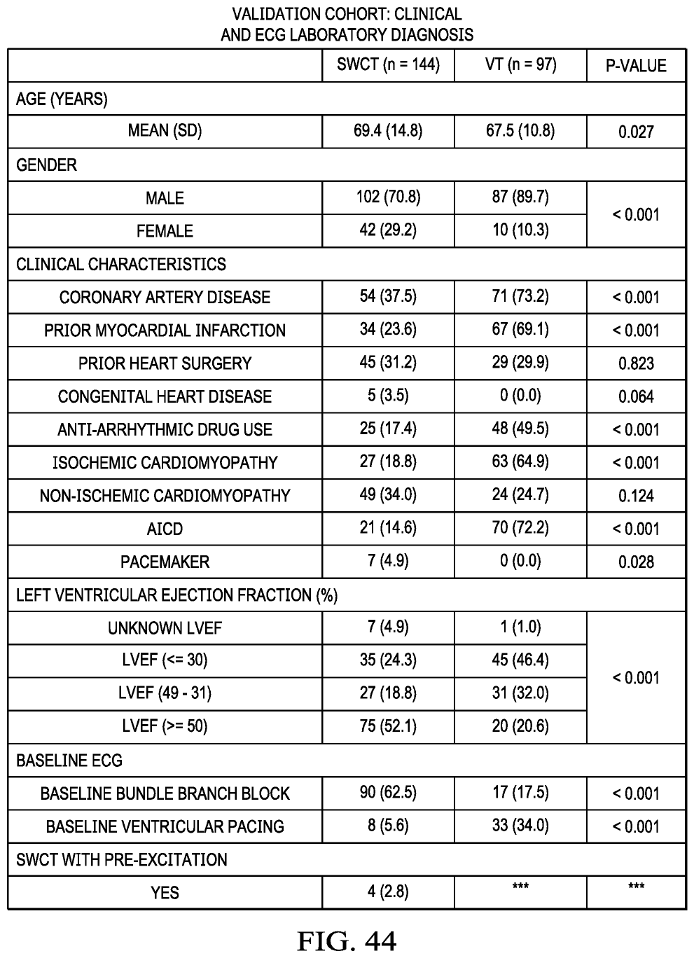

Patient characteristics of VT and SWCT groups for the validation cohort are summarized in FIG. 44. The VT group included more ECG pairs from patients with coronary artery disease, prior MI, ongoing antiarrhythmic drug use, ischemic cardiomyopathy, and an implanted AICD. The VT group comprised more ECG pairs from patients with reduced LVEF (<50%), whereas the SWCT group included more ECG pairs from patients with preserved LVEF (>=50%). Baseline ECGs with ventricular pacing were more prevalent in the VT group, whereas baseline BBB was more common in the SWCT group. Four SWCTs demonstrated atrioventricular pre-excitation.

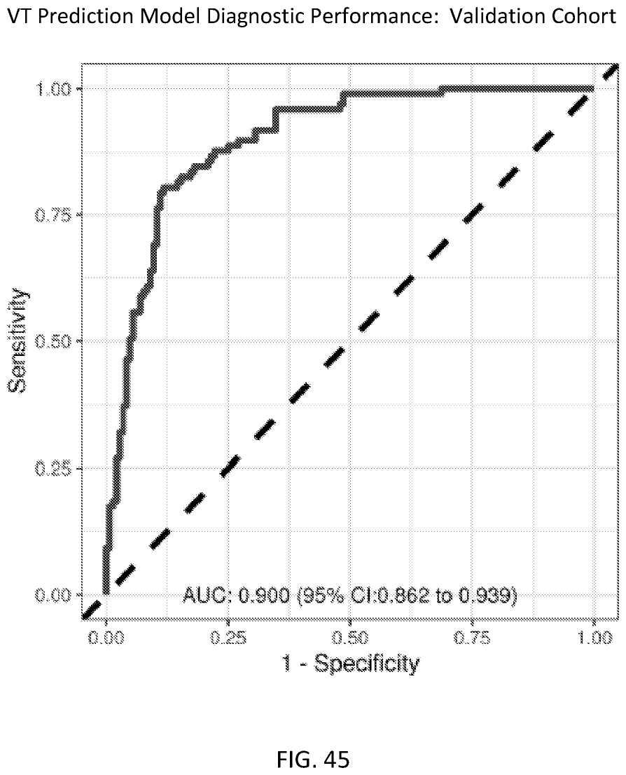

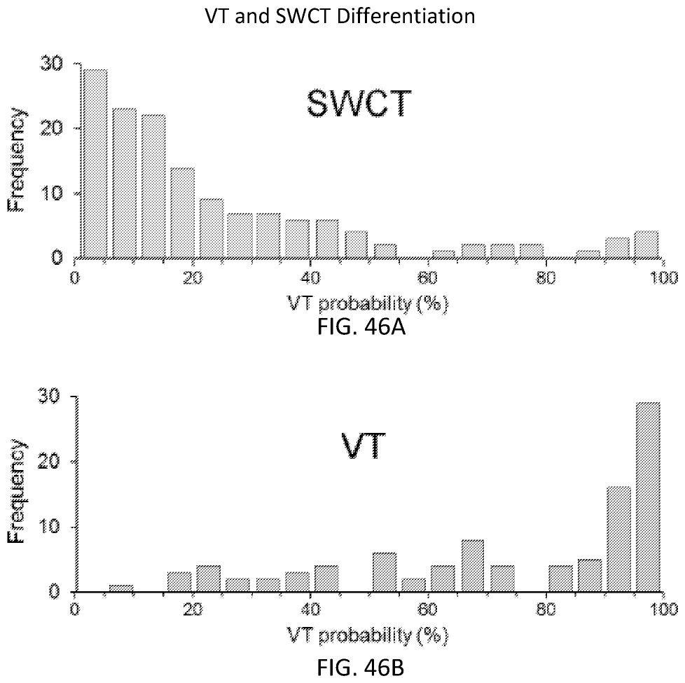

The VT Prediction Model yielded effective VT and SWCT differentiation (AUC 0.900; CI 0.862-0.939) when implemented on the validation cohort (FIGS. 45 and 46). When using a 50% VT probability partition (VT>=50%; SWCT<50%), overall accuracy, sensitivity and specificity was 85.0%, 80.4% and 88.2%, respectively. Overall accuracy did differ among patients with (77.1%) or without (89.2%) an accompanying electrophysiology procedure (p=0.02). Notably, the VT Prediction Model and ECG laboratory did not differ in their degree of agreement with clinical diagnosis (p=0.87).

The VT Prediction Model accurately segregated the majority of clinical VTs (55.7%) and SWCTs (67.4%) as having higher (>=75%) or lower (<25%) VT probabilities, respectively. Additionally, a sizeable proportion of VTs (46.3%) were correctly categorized as having high VT probability (=>90%) with a positive predictive value of 86.5%. Four clinical SWCTs (2.8%) erroneously assigned high VT probability (>=90%) were derived from a single patient with ventricular pre-excitation across a left lateral atrioventricular accessory pathway. In an opposite manner, a sizeable proportion of SWCTs (36.1%) were appropriately categorized as having low VT probability (<10%) with a negative predictive value of 98.1%. Only one clinical VT (1.0%) was erroneously assigned low VT probability (<10%).

The VT Prediction Model accurately differentiates the large majority of WCTs expected to be encountered in clinical practice. Moreover, despite the presumably persuasive influence that an ECG laboratory diagnosis may have on patients' final clinical diagnosis the VT Prediction Model agreed with patients' clinical diagnosis just as well as ECG interpretations provided by supervising cardiologies interpreters.

The VT Prediction Model generates a continuum of VT probabilities that demonstrate favorable diagnostic performance indices across a wide variety of VT probability partitions. For example, when using a 50% VT probability partition (VT>=50%; SWCT<50%), the VT Prediction Model accurately distinguishes most WCTs (84.9%) with a diagnostic sensitivity (80.6%) and specificity (88.4%) for VT. Alternatively, when using a 25% VT probability partition (VT>=25%; SWCT<255), the VT Prediction Model yields stronger sensitivity (90.8%) for VT while maintaining satisfactory preservation of overall accuracy (83.2%) and specificity (76.8%) for VT.

Owing to less efficient ventricular depolarization, VT ordinarily expresses longer QRS durations than SWCT. This dissimilarity was originally verified by Wellens and co-workers in 1978 (3), and has since inspired proposed QRS duration cut-offs to determine VT diagnoses: QRS>140 ms for WCTs with right bundle branch block (BBB) configuration and QRS>160 ms for WCTs with left BBB configuration (14). Unfortunately, due to considerable QRS duration range overlap, VT and SWCT discrimination cannot be confidently accomplished using only WCT QRS duration cut-offs. A substantial proportion of SWCTs will demonstrate QRS durations greater than 160 ms, especially when they develop among patients with ongoing anti-arrhythmic drug use, preexisting BBB and/or advanced cardiomyopathy. On the other hand, VTs may periodically demonstrate QRS durations less than 140 ms if they arise from patients without structural heart disease and/or originate within or near the His-Purkinje network.

It was observed that WCTs exhibiting shorter QRS durations were more likely SWCT, while WCTs demonstrating longer QRS durations were more likely VT. Furthermore, we observed WCT QRS duration affects a graded increase or decrease in VT likelihood—WCT QRS prolongation increases VT probability, whereas shortened WCT QRS durations decrease VT probability.

Wide complex tachycardia onset or offset typically produces a concomitant change in QRS duration. In many cases, large QRS duration changes are due to major differences in the means of ventricular depolarization. For example, following the onset of VTs originating well outside the normal conduction system, a large increase in QRS duration will be evident if the baseline heart rhythm exhibits rapid, efficient ventricular depolarization via a healthy His-Purkinje network. In contrast, WCTs that demonstrate similar means of ventricular depolarization as the baseline heart rhythm tend to express minimal QRS duration changes. For example, SWCTs that utilize the same conduction system or implanted intracardiac device pathways as the baseline heart rhythm often express minimal QRS duration changes. On the other hand, “functional” SWCTs, which express newfound ventricular depolarization delays, characteristically generate an increase in QRS duration.

It was observed that QRS duration changes between patients' baseline and WCT ECG helps differentiate WCTs—smaller QRS duration changes predict SWCT, while larger QRS duration changes predict VT. Interestingly, this trend was observed among patients with and without QRS duration prolongation (=>120 ms) on their baseline ECG. That is, our findings suggest VTs normally produce greater QRS duration changes than SWCTs arising from preexisting and functional aberrancy.

Wide complex tachycardia onset or offset may produce large or small changes in the mean electrical vector of ventricular depolarization. Provided that the means by which VT may propagate within and depolarize the ventricular myocardium is virtually unlimited, VT may express an expansive variety of mean electrical vectors with differing direction (i.e. mean electrical axis) and/or voltage intensity dissimilar to the respective baseline heart rhythm. In contrast, the manner by which SWCTs depolarize the ventricular myocardium is ordinarily confined to the same His-Purkinje network or intracardiac pacing delivery system utilized by the baseline heart rhythm. Only in rare circumstances are SWCTs due to ventricular pre-excitation arising from separate atrioventricular accessory pathways. Therefore, many SWCTs, especially those with preexisting aberrancy or ventricular pacing, express similar mean electrical vectors compared their respective baseline ECG. Conversely, SWCTs with functional aberration typically express more substantial changes in the direction and/or magnitude of the mean electrical vector. However, given functional SWCTs arise from antegrade impulse propagation and ventricular depolarization confined within the His-Purkinje network, they are destined to express a more constrained variety of mean electrical vectors. As a consequence, SWCTs, including those due to functional aberrancy, are expected to demonstrate smaller changes to the mean electrical axis than VT. In a similar manner, VT is anticipated to exhibit larger changes in the mean electrical axis.

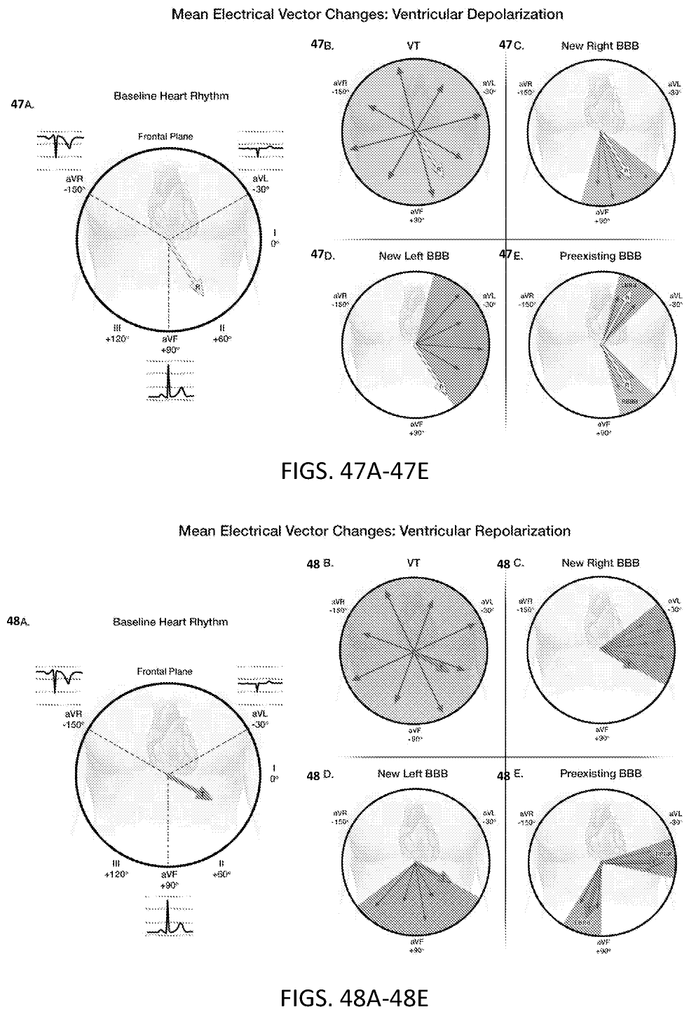

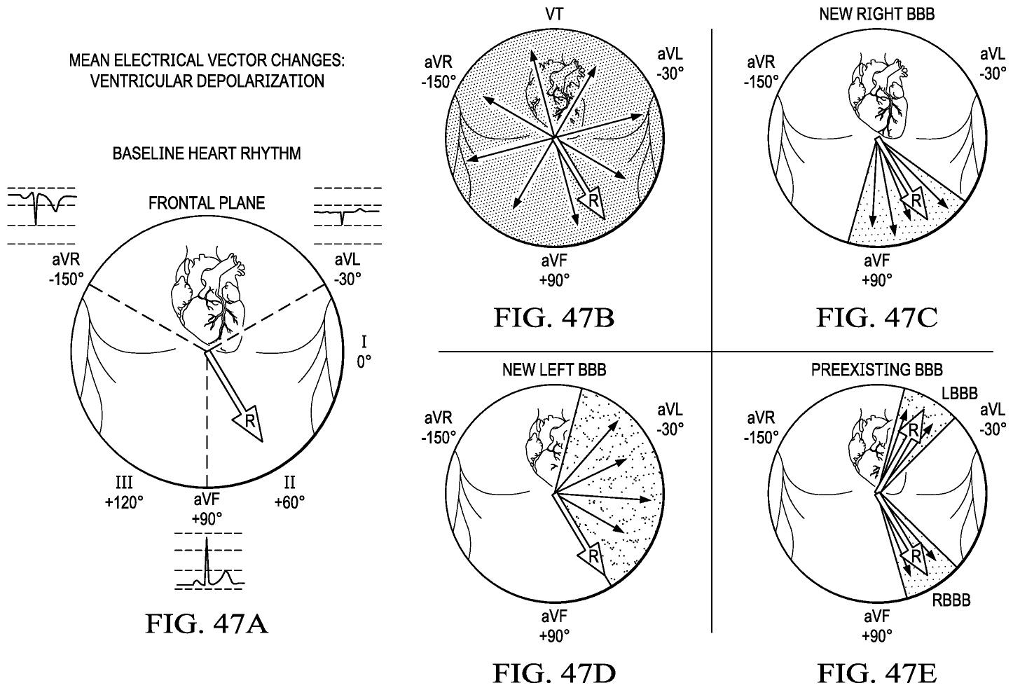

The extent of mean electrical vector changes that occur upon WCT onset or offset are primarily influenced by the underlying WCT rhythm. Through an analysis of paired WCT and subsequent baseline ECGs, it was confirmed that VT normally generates greater QRS amplitude changes (i.e. frontal and horizontal percent amplitude change) than SWCT. Given that QRS amplitude changes correspond directly to changes in the mean electrical vector of ventricular depolarization, we soundly concluded VT normally expresses greater mean electrical vector changes than SWCT. Since QRS axis (or “R” axis on ECG paper recordings) represents the direction for the mean electrical vector of ventricular depolarization, it is anticipated that VT would commonly express greater QRS axis changes than SWCT. FIGS. 47A-47E depict panels summarizing the expected changes to the mean electrical vector of ventricular depolarization following WCT initiation. Displayed arrows represent mean electrical vectors for ventricular depolarization within the frontal ECG plane. The directional orientation of individual arrows represents the mean electrical axis of ventricular depolarization (i.e., QRS axis). Heavy yellow arrows represent the baseline heart rhythm's mean electrical vector for ventricular depolarization (the inscribed “R” signifies “R axis” [i.e. QRS axis] displayed on ECG paper recordings). Color-shaded regions represent the expected range of mean electrical vectors after WCT onset. Panel 47A depicts the mean electrical vector of ventricular depolarization for a normal baseline heart rhythm. Panels 47B-47E depict various examples of expected changes in the mean electrical vector of depolarization that occur upon WCT initiation. VT (Panel 47B) demonstrates an expansive range of possible mean electrical vectors. SWCTs due to functional RBBB (Panel 47C) or LBBB (Panel 47D) exhibit a constrained range of potential mean electrical vectors. SWCTs arising from BBB (Panel 47E) demonstrate minimal mean electrical vector changes.

The degree of QRS axis change was identified as strong, independent VT predictor—VT commonly generated greater QRS axis changes than SWCT. It was observed that this distinction persisted irrespective of baseline ECG subgroupings: QRS duration >=120 ms, QRS duration <120 ms or ventricular pacing. Furthermore, the degree of QRS axis shifts contributes to a corresponding probabilistic change in VT likelihood. That is, large QRS axis changes yield an increase in VT probability, whereas smaller QRS axis shifts generate correspondingly lower VT probability.

These findings complement the observations originally put forth by Griffith and co-workers in 1985 (37). In their study, they identified large QRS axis shifts (>=40°) from the baseline sinus rhythm ECG to be the 3^{rd} strongest independent VT predictor (behind MI history and lead aVF QRS configuration) among 15 clinical and 11 electrocardiographic variables. In this present analysis, the degree of QRS axis change (range: 0°-180°) is further quantified so to provide an exact estimation of VT probability.

T axis change was identified as a strong independent VT predictor—larger T axis changes predict VT, while smaller T axis changes predicted SWCT. This pattern was observed among patients with and without baseline ECG QRS duration prolongation (=>120 ms). Moreover, similar to QRS axis changes, the extent of T axis change contributed to a graded increase or decrease in VT likelihood. That is, larger T axis changes conferred a corresponding increase in VT probability, while smaller T axis changes yielded decreased VT probability.

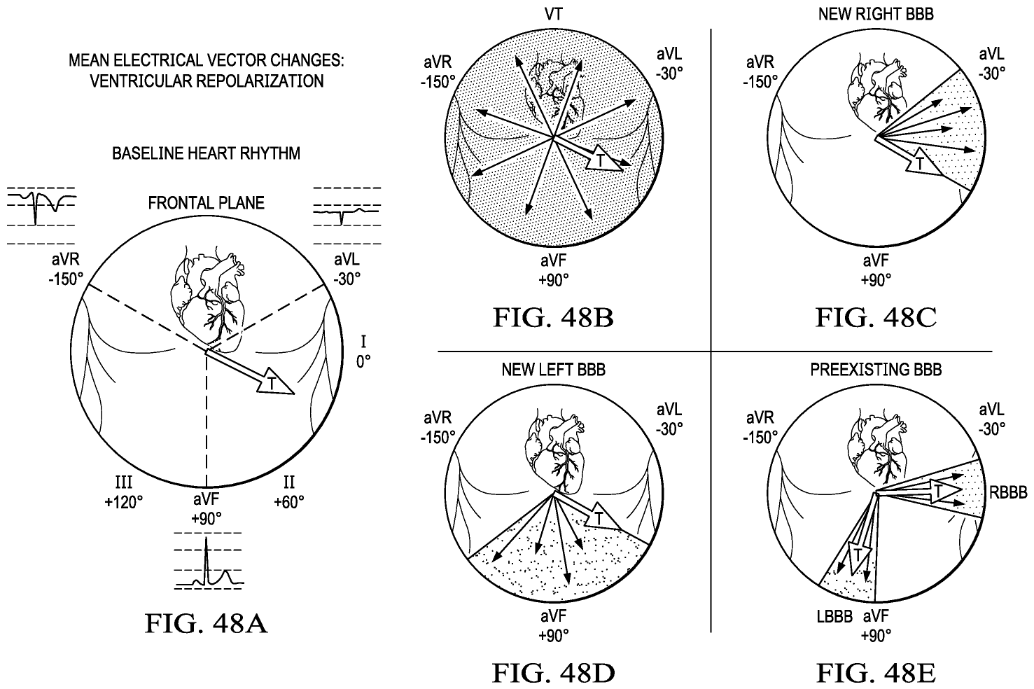

It is believed that this is the first study to reveal directional changes in the mean electrical axis of ventricular repolarization helps distinguish VT and SWCT. Although the electrophysiologic basis for this observation has yet to be fully characterized, we hypothesize changes in the electromotive forces of ventricular repolarization, occurring upon WCT onset or offset, are strongly influenced by the underlying WCT rhythm. FIGS. 48A-48E depict panels summarizing expected changes to the mean electrical vector of ventricular repolarization upon WCT initiation. Displayed arrows represent mean electrical vectors for ventricular repolarization in the frontal ECG plane. The directional orientation of individual arrows represents the mean electrical axis of ventricular repolarization (i.e., T axis). Heavy orange arrows represent the baseline heart rhythm's mean electrical vector for ventricular repolarization (the inscribed “T” signifies “T axis” displayed on ECG paper recordings). Color-shaded regions represent the expected range of mean electrical vectors after WCT onset. Panel 48A depicts the mean electrical vector of ventricular depolarization and repolarization for a normal baseline heart rhythm. Panels 46B-46E depict various examples of expected changes in the mean electrical vector of repolarization that occur upon WCT initiation. VT (Panel 48B) demonstrates an expansive range of possible mean electrical vectors. SWCTs due to functional RBBB (Panel 48C) or LBBB (Panel 48D) exhibit a constrained range of potential mean electrical vectors. SWCTs arising from BBB (Panel 48E) demonstrate minimal mean electrical vector changes. Therefore, in much the same way as changes in the mean electrical axis of depolarization (i.e. QRS axis change), quantifying the degree of change in the mean electrical axis of ventricular repolarization (i.e. T axis change) provides a means to correctly differentiate WCTs.

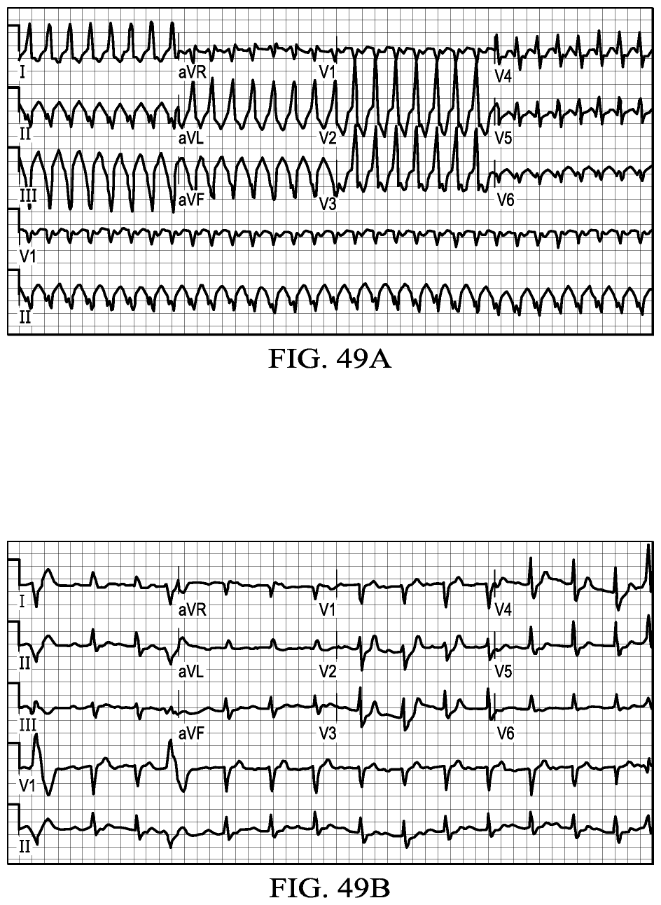

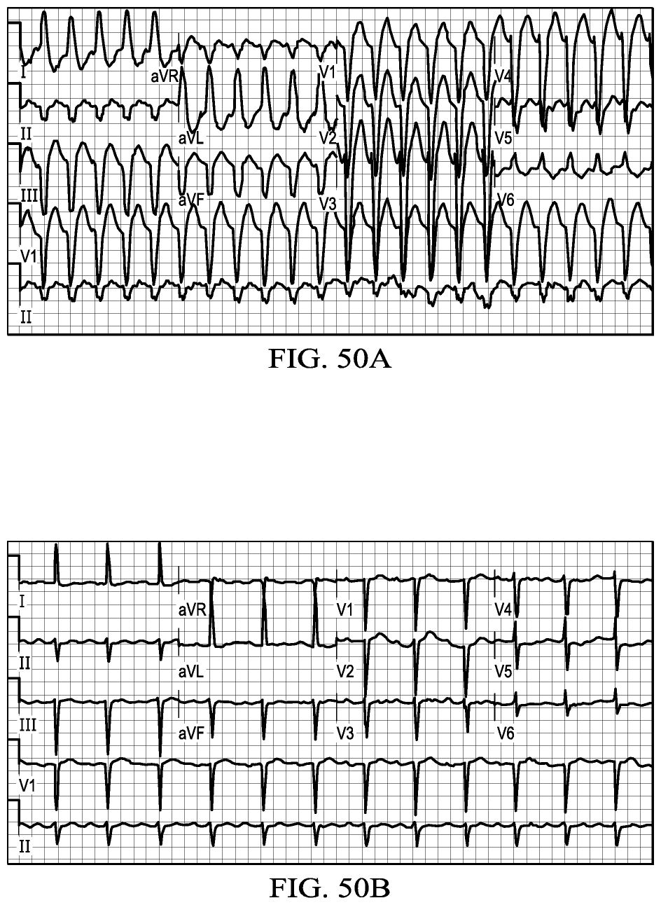

According to the VT Prediction Model's structure, “actual” VTs may be erroneously classified as SWCT if they demonstrate narrow QRS durations (e.g. fascicular VT), minimal QRS duration changes (e.g., VT arising from a baseline ventricular paced rhythm) and/or similar ventricular depolarization and/or repolarization patterns compared to the baseline ECG (e.g. bundle branch re-entry). Correspondingly, it was observed that erroneous SWCT predictions for clinical VTs that demonstrate narrower QRS durations and/or minimal changes in QRS duration, QRS axis, and/or T axis compared to the baseline ECG. FIGS. 49A-47B depict examples of paired VT (FIG. 49A) and baseline (FIG. 49B) ECGs assigned low VT probability (9.8704%) by the VT Prediction Model (WCT QRS duration=126 ms; QRS duration change=6 ms; QRS axis change=63°; T axis change=30°). On the other hand, the VT Prediction Model may erroneously classify “actual” SWCTs as VT if they express wider QRS durations (e.g. QRS prolongation due to antiarrhythmic drugs), large QRS duration changes (e.g. functional right or left BBB) and/or pronounced changes in ventricular depolarization and/or repolarization (e.g. functional left BBB and ventricular pre-excitatation). Accordingly, it was observed that erroneous VT predictions for clinical SWCTs exhibiting wider QRS durations and/or large changes in QRS duration, QRS axis, and/or T axis compared to the baseline ECG. FIGS. 50A-50B depict examples of paired SWCT (FIG. 50A) and baseline (FIG. 50B) ECGs assigned high VT probability (54.0039%) by the VT Prediction Model (WCT QRS duration=170 ms; QRS duration change=52 ms; QRS axis change=3°; T axis change=89°).

Successful application of the VT Prediction Model requires computerized measurements from paired WCT and baseline ECG recordings. In circumstances where WCT patients present without a previously acquired and electronically archived baseline ECG, interpreters must temporarily rely on alternative means to differentiate WCTs until a subsequent baseline ECG is recorded.

The VT Prediction Model is a prototypical example of how to successfully differentiate WCTs without relying upon the manual application of traditional ECG criteria or algorithms. The VT Prediction Model's implementation merely requires the input of computerized ECG measurements routinely displayed on 12-lead ECG paper recordings: QRS duration, QRS axis (i.e. “R” axis) and T axis. In this manner, three universal ECG measurements displayed on WCT and baseline ECGs—recorded before or after the WCT event—may be readily used to deliver an estimation of VT probability. A similar approach may be performed by successive iterations that use more sophisticated modelling techniques (e.g., artificial neural networks).

Various VT Prediction Model embodiments would be well-suited to provide diagnostic assistance to ECG interpreters—especially those who struggle to accurately differentiate WCTs with manual ECG interpretation methods. For example, online calculators and/or mobile device applications, implementing the VT Prediction Model to deliver an unambiguous estimation of VT probability, may help clinicians commit to (or reconsider) VT or SWCT diagnoses reached by other WCT differentiation methods. Alternatively, such tools may be used as the principle means to differentiate WCTs, especially for ECG pairs assigned high (>=90%) or low (<10%) VT probability. In this manner, the VT Prediction Model (and its successive interations) would have a tangible means to deliver clinicians a cognitively meaningful estimation of VT probability.

In order to provide an accurate representation of WCTs customarily encountered in clinical practice, clinical WCTs formally diagnosed by the institution's ECG laboratory and physicians were evaluated. In so doing, approximately one-third of selected WCTs were derived from patients who underwent an electrophysiology study. Although it was observed that the VT Prediction Model's overall performance did not differ when implemented on patients with and without an electrophysiology procedure during model derivation, a difference in model performance was detected during model validation.

Variations in ECG electrode placement and/or major changes to the patient's baseline ECG (e.g. new ventricular pacing) may have been able to negatively influence the VT Prediction Model's performance. Despite not being intentionally excluded, no SWCTs exhibiting atrioventricular pre-excitation were evaluated. Paired ECGs used to derive the VT Prediction Model were acquired from patients presenting to the tertiary medical center and surrounding health system. The VT Prediction Model's diagnostic performance was not directly compared conventional WCT differentiation methods (6, 8, 10, 12, 14, 15).

The VT Prediction Model is a prototypical exemple of how universal computerized ECG measurements may be utilized to create a simplified, user-friendly means to differentiate WCTs. This approach to WCT diagnosis has the natural advantage of providing definite estimations of VT probability irrespective of clinicians' competency using manually-applied WCT criteria or algorithms. The VT Prediction Model can be applied to any standard 12 lead ECG performed. Thus, those performed in the outpatient office, inpatient setting, on remotely worn devices may utilize the VT Prediciton Model This can also include application to 12 lead ECGs, Holter monitoring, or other wearable devices. Moreover, the VT Prediction Model can be directly incorporated into ECG interpretation software (e.g., MUSE by GE Healthcare, etc.). QRS duration (ms), R wave axis (°) and T wave (°) can be derived from as few as two leads (e.g., Lead I and III). This feature increases the diagnostic utility and bandwidth of utility of this method in more general settings, such as rural hospitals or in intensive care units where 2-3 leads are commonly used for continuous heart rhythm monitoring and provide an automated means of detection for any care setting. A similar operation may be employed using QRS duration, QRS axis and T wave axis derived from EMG data, a VCG data, and/or a mathematically-synthesized VCG data. Moreover, comparable models may ultilize an interface that can be downloaded to a patient's smartphone such as an mobile device application, via a website for clinicians with access to a computer, or on wireless ECG based interpretation systems—i.e., used by ambulance, ICU, cath labs, or ECG hospital monitoring based platforms so that an automated alert is sent to a member of a care team in order to expedite workup and evaluation for a given patient.

Although a logistic regression model was used above to demonstrate the accuracy of the VT prediction model, other machine learning models can be used, such as neural networks, Random Forests, K-nearest neighbors, support vector machines, ect. Moreover, additional inputs or parameters can be used to adapt or modify the VT prediction model. For example, there are several permutations of higher order and magnitude that can be more complex and thus provide even more robust data and enhanced accuracy. These can include training data sets which become “smarter” for each patient or collection of patients to offer a refining model—i.e. improvement through learning steps associated from augmented intelligence from a fed-in learning set. This can include but is not limited to several features for VT probability prediction or VT/SWCT classification models. For example, such augmented intelligence techniques can provide personalized models via several ECGs with “WCT” versus “normal” ECGs that could correctly inform/notify providers of relapsed VT or SWCT and to allow an even more precise/accurate read out of an ECG in an automated fashion. As a result, the VT Prediction model (or its successive iterations or other predictive modelling variants) can be applied by computerized ECG interpretation software systems in multiple permutations, tailored for specific patient populations, and applied in multiple interfaces.

Now referring back to FIG. 29, an apparatus 2900 for classifying a WCT heat beats in accordance with the present invention is shown. The apparatus 2900 can be a server computer, a workstation computer, a laptop computer, a mobile communications device, a personal data assistant, a medical device or any other device capable of performing the functions described herein. The apparatus 2900 includes an input/output interface 2902, a memory 2904, and one or more processors 2906 communicably coupled to the input/output interface 2902 and the memory 2904. Note that the apparatus 2900 may include other components not specifically described herein. The memory 2904 can be local, remote or distributed. Likewise, the one or more processors 2906 can be local, remote or distributed. The input/output interface 2902 can be any mechanism for facilitating the input and/or output of information (e.g., web-based interface, touchscreen, keyboard, mouse, display, printer, etc.) Moreover, the input/output interface 2902 can be a remote device communicably coupled to the one or more processors 2906 via one or more communication links 2908 (e.g., network(s), cable(s), wireless, satellite, etc.). The one or more communication links 2908 can communicably couple the apparatus 2900 to other devices 2910 (e.g., databases, remote devices, hospitals, doctors, researchers, patients, etc.).

The one or more processors 2906 receive a wide complex heart beat data comprising at least a wide complex heart beat QRS duration, a wide complex heart beat R wave axis and a wide complex heart beat T wave axis via the input/output interface 2902 or the memory 2904; receive a baseline heart beat data comprising at least a baseline heart beat QRS duration, a baseline heart beat R wave axis, and a baseline heart beat T wave axis via the input/output interface 2902 or the memory 2904; determine a signal change between the wide complex heart beat data and the baseline heart beat data areas; and provide the signal change via the input/output interface 2902, wherein the signal change provides an indication whether the wide complex heart beat(s) is from a ventricular source or a supraventricular source.

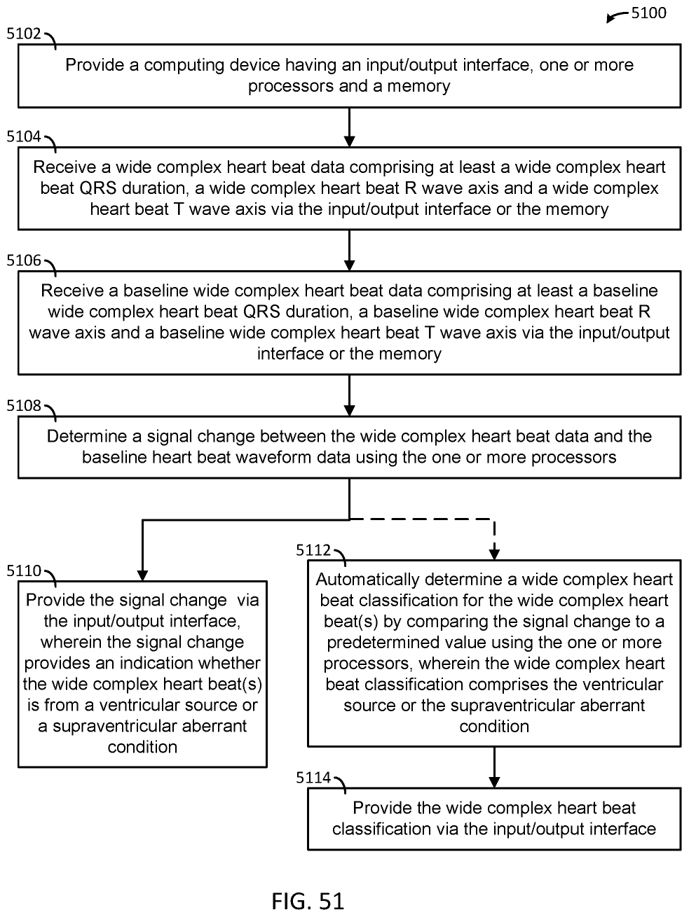

Referring now to FIG. 51, a flow chart of a computerized method 5100 of automatically classifying a wide complex heart beat(s) is shown. A computing device having an input/output interface, one or more processors and a memory is provided in block 5102. A wide complex heart beat data comprising at least a wide complex heart beat QRS duration, a wide complex heart beat R wave axis and a wide complex heart beat T wave axis is received via the input/output interface or the memory in block 5104. A baseline heart beat data comprising at least a baseline heart beat QRS duration, a baseline heart beat R wave axis, and a baseline heart beat T wave axis is received via the input/output interface or the memory in block 5106. A signal change between the wide complex heart beat data and the baseline heart beat data areas is determined using the one or more processors in block 5108. The signal change is provided via the input/output interface in block 5110, wherein the signal change provides an indication whether the wide complex heart beat(s) is from a ventricular source or a supraventricular source. In another embodiment, a wide complex heart beat classification for the wide complex heart beat(s) is automatically determined by comparing the signal change to a predetermined value using the one or more processors in block 5112, wherein the wide complex heart beat classification comprises the ventricular source or the supraventricular source. The wide complex heart beat classification is provided via the input/output interface in block 5114. The signal change in ventricular repolarization can be concomitantly “weighted” with other predictors of VT, SWCT or ventricular pacing. Moreover, the method can be implemented using a non-transitory computer readable medium that when executed causes the one or more processors to perform the method.

Now referring to FIGS. 29 and 51, other aspects of the present invention that are applicable to the apparatus 2900 and the method 5100 will now be described. In one aspect, the signal change in ventricular repolarization further provides the indication whether the wide complex heart beat(s) is due to ventricular pacing or a premature ventricular contraction (PVC). In another aspect, the wide complex heart beat(s) comprise a wide complex tachycardia (WCT), the ventricular source comprises a ventricular tachycardia (VT), and the supraventricular source comprises a supraventricular wide complex tachycardia (SWCT). In another aspect, the signal change in ventricular repolarization comprises a VT probability, the wide complex heart beat classification comprises a VT whenever the VT probability is greater than or equal to the predetermined value, and the wide complex heart beat classification comprises a SWCT whenever the VT probability is less than the predetermined value. In another aspect, the one or more processors select the predetermined value from a range of 0% to 100%. In another aspect, the predetermined value comprises about 1%, 10%, 25%, 50%, 75%, 90% or 99%. In another aspect, the one or more processors provide the signal change in ventricular repolarization by providing a “shock” recommendation signal, a “no shock” recommendation signal, or no signal. In another aspect, the wide complex heart beat data and the baseline heart beat waveform data are obtained from an electrocardiogram (ECG) signal, a ventricular electrogram (EMG) signal, a vectorcardiogram (VCG), and/or a mathematically-synthesized VCG signal.

In another aspect, the one or more processors: receive the wide complex heart beat data by receiving a ECG QRS data, a EMG data, a VCG data, and/or a mathematically-synthesized VCG data via the input/output interface or the memory, and determine the wide complex heart beat data from the ECG QRS data, the EMG data, the VCG data and/or the mathematicallly-synthesized VCG data; and receive the baseline heart beat data by receiving a baseline ECG QRS data, a baseline EMG data, a baseline VCG data, and/or a baseline mathematically-synthesized VCG data via the input/output interface or the memory, and determine the baseline heart beat data from the baseline ECG QRS data, the baseline EMG data, the baseline VCG data and/or the baseline mathematically-synthesized VCG data. In another aspect, the ECG data, the EMG data, the VCG data and/or the mathematically-synthesized VCG data is generated or recorded before or after the baseline ECG data, the baseline EMG data, the baseline VCG data and/or the baseline mathematically-synthesized VCG data. In another aspect, the ECG data, the EMG data, the VCG data and/or the mathematically-synthesized VCG data is generated or recorded after the baseline ECG data, the baseline EMG data, the baseline VCG data and/or the baseline mathematically-synthesized VCG data and determining the signal change. In another aspect, the ECG data, the EMG data, the VCG data and/or the mathematically-synthesized VCG data and the baseline ECG data, the baseline EMG data, the baseline VCG data and/or the baseline mathematically-synthesized VCG data are generated or recorded using one or more sensors or devices. In another aspect, the one or more sensors or devices comprise two or more leads of a ECG device, a 12-lead ECG device, a continuous ECG telemetry monitor, a stress testing system, an extended monitoring device, a smartphone-enabled ECG medical device, a cardioverter-defibrillator therapy device, a subcutaneous implantable cardioverter defibrillators (ICD), an intracardiac or transvenous pacemaker device, an automated external defibrillators (AED), or an automatic implantable cardioverter defibrillator (AICD). In another aspect, the input/output interface, the memory and the one or more processors are integrated into the one or more sensors or devices; or the one or more sensors or devices are integrated into a computing device comprising the input/output interface, the memory and the one or more processors.

In another aspect, the signal change comprises a classification probability comprising a VT probability, a SWCT probability, ventricular pacing probability or a ventricular pacing probability. In another aspect, the one or more processors determine the classification probability based one or more additional classification predictors. In another aspect, the one or more processors determine the signal change by determining a VT probability using a statistical or machine learning process. In another aspect, the statistical or machine learning process comprises a linear regression algorithm, a logistic regression model, a linear discriminate analysis algorithm, a Naive Bayes algorithm, a computational model using artificial neural networks, a computational model based on classification or regression trees, a k-nearest neighbors based model, a support vector machine based model, a boosting algorithm, or an ensemble machine learning algorithm. In another aspect, the one or more processors determine the signal change by determining a VT probability (P_{VT}) by:

$${P}_{VT} = \frac{{e}^{( a + b \times ( R axis change ) + c \times ( T axis change ) + d \times ( WCT QRS duration ) + g \times ( QRS duration change ) )}}{\begin{matrix}1 + \\ {e}^{( a + b \times ( R axis change ) + c \times ( T axis change ) + d \times ( WCT QRS duration ) + g \times ( QRS duration change ) )}\end{matrix}} ,$$

where: a, b, c, d and g are constants,
the QRS axis change=an absolute or non-absolute value of the wide complex heart beat R wave axis minus the baseline heart beat R wave axis,
the T axis change=an absolute or non-absolute value of the wide complex heart beat T wave axis minus the baseline heart beat T wave axis,
the WCT QRS duration=the wide complex heart beat QRS duration,
the QRS duration change=an absolute or non-absolute value of the wide complex heart beat QRS duration minus the baseline heart beat QRS duration.

In another aspect, the input/output interface comprises a remote device, and the remote device is communicably coupled to the one or more processors via one or more networks. In another aspect, the one or more processors receive the wide complex heart beat data by monitoring a person using one or more sensors or devices communicably coupled to the input/output interface. In another aspect, the one or more processors provide a recommendation to select or exclude a therapy, medication, diagnostic testing or referral for a patient based on the signal change in ventricular repolarization. In another aspect, the one or more processors send an alert to one or more devices in based on the signal change. In another aspect, the one or more processors count the frequency of various wide complex beats comprising ventricular tachycardia events, supraventricular tachycardia events, singular supraventricular wide complex beats, premature ventricular contraction, right ventricular pacing and/or biventricular pacing. In another aspect, the one or more processors receive multiple sets of the wide complex heart beat data and the baseline heart beat data for a person or group of persons, determine the signal change for each set of the wide complex heart beat data and the baseline heart beat data for the person or the group of persons, and create a VT prediction model for the person or the group of persons using the signal changes. In another aspect, apparatus comprises a server computer, a workstation computer, a laptop computer, a mobile communications device, a personal data assistant, or a medical device.

Ventricular Repolarization Embodiments

The following types of embodiments of the present invention provide a WCT differentiation method that is based in whole or in part on an analysis of ventricular repolarization. This alternative approach to diagnosis has the natural advantage of automatically delivering precise estimations of VT probability to clinicians irrespective of their ECG interpretation abilities. For example, automated methods incorporating T wave amplitude based and T wave time-voltage area based parameters are particularly well-suited to help providers with less experience and/or differing clinical expertise provide accurate and timely WCT diagnoses. The incorporation of the present invention into computerized ECG interpretation software systems will not only supplement current diagnostic strategies but may also improve the quality of care provided to patients with WCT. In addition, when added to other known discriminators of VT and SWCT (e.g., percent change in QRS amplitude), the incorporation of T wave amplitude based and T wave time-voltage area based parameters will augment the differentiating capacity of predictive models.

Additionally, the electrophysiological principles described herein may be applied to a wide variety of ECG, EMG, VCG, and/or mathematically-synthesized VCG analysis platforms beyond the diagnostic 12-lead ECG, including continuous ECG telemetry monitors, stress testing systems, extended monitoring devices (e.g., Holter monitors, etc.), smartphone-enabled ECG medical devices, cardioverter-defibrillator therapy devices, such as wearable cardioverter defibrillators (e.g., Zoll Life Vest), subcutaneous implantable cardioverter defibrillators (ICD) (e.g., Emblem S-ICD by Boston Scientific), intracardiac or transvenous pacemaker devices, automated external defibrillators (AED) (e.g., HeartStart OnSite AED by Phillips), and conventional automatic implantable cardioverter defibrillators (AICD). Measurements and calculations of EMG signals recorded from intracardiac (e.g. right ventricular AICD coil) and/or extracardiac electrodes (e.g. AICD generator housing) may also be used to established the degree (or percentage) change in ventricular repolarization. For example, amplitude or time-voltage area changes in ventricular repolarization between the WCT and baseline ventricular EMGs to help distinguish VT and SWCT. This discrimination process could be used to determine the need to deliver of device-related therapies, including anti-tachycardia pacing and defibrillator shocks.

Now referring back to FIG. 29, an apparatus 2900 for classifying a WCT heartbeats in accordance with the present invention is shown. The apparatus 2900 can be a server computer, a workstation computer, a laptop computer, a mobile communications device, a personal data assistant, a medical device or any other device capable of performing the functions described herein. The apparatus 2900 includes an input/output interface 2902, a memory 2904, and one or more processors 2906 communicably coupled to the input/output interface 2902 and the memory 2904. Note that the apparatus 2900 may include other components not specifically described herein. The memory 2904 can be local, remote or distributed. Likewise, the one or more processors 2906 can be local, remote or distributed. The input/output interface 2902 can be any mechanism for facilitating the input and/or output of information (e.g., web-based interface, touchscreen, keyboard, mouse, display, printer, etc.) Moreover, the input/output interface 2902 can be a remote device communicably coupled to the one or more processors 2906 via one or more communication links 2908 (e.g., network(s), cable(s), wireless, satellite, etc.). The one or more communication links 2908 can communicably couple the apparatus 2900 to other devices 2910 (e.g., databases, remote devices, hospitals, doctors, researchers, patients, etc.).

The one or more processors 2906 receive one or more wide complex heart beat waveform amplitudes and/or time-voltage areas of ventricular repolarization, and one or more baseline heart beat waveform amplitudes and/or time-voltage areas of ventricular repolarization via the input/output interface 2902 or the memory 2904, determine a signal change in ventricular repolarization between the wide complex heart beat waveform amplitudes and/or time-voltage areas of ventricular repolarization and the baseline heart beat waveform amplitudes and/or time-voltage areas of ventricular repolarization, and provide the signal change in ventricular repolarization via the input/output interface 2902, wherein the signal change in ventricular repolarization provides an indication whether the wide complex heart beat(s) is from a ventricular source or a supraventricular source. In one embodiment, the delivery of the signal change in ventricular repolarization, such as % VT probability, to clinicians provides an practical diagnostic tool that allows them to use their clinical judgement as how to manage the patient. In another embodiment, the one or more processors 2906 provide the signal change in ventricular repolarization via the input/output interface 2902 by automatically determining a wide complex heart beat classification for the wide complex heart beat(s) by comparing the signal change in ventricular repolarization to a predetermined value using the one or more processors, and providing the wide complex heart beat classification via the input/output interface 2902.

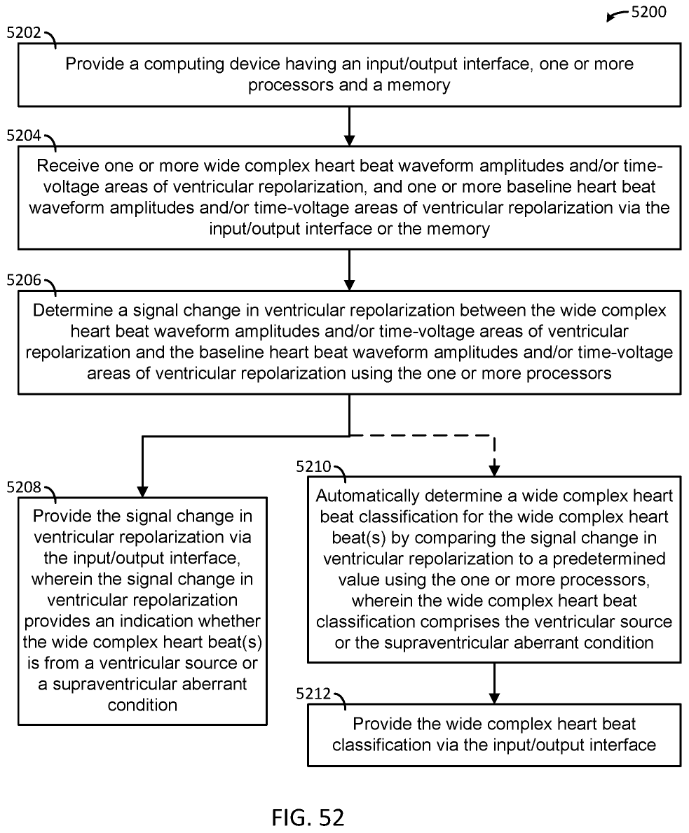

Referring now to FIG. 52, a flow chart of a computerized method 5200 of automatically classifying a wide complex heart beat(s) is shown. A computing device having an input/output interface, one or more processors and a memory is provided in block 5202. One or more wide complex heart beat waveform amplitudes and/or time-voltage areas of ventricular repolarization, and one or more baseline heart beat waveform amplitudes and/or time-voltage areas of ventricular repolarization are received via the input/output interface or the memory in block 5204. A signal change in ventricular repolarization between the wide complex heart beat waveform amplitudes and/or time-voltage areas of ventricular repolarization and the baseline heart beat waveform amplitudes and/or time-voltage areas of ventricular repolarization is determined using the one or more processors in block 3006. The signal change in ventricular repolarization is provided via the input/output interface in block 5208, wherein the signal change in ventricular repolarization provides an indication whether the wide complex heart beat(s) is from a ventricular source or a supraventricular source. In another embodiment, a wide complex heart beat classification for the wide complex heart beat(s) is automatically determined by comparing the signal change in ventricular repolarization to a predetermined value in block 5210, wherein the wide complex heart beat classification comprises the ventricular source or the supraventricular source. The wide complex heart beat classification is provided via the input/output interface in block 5212. The signal change in ventricular repolarization can be concomitantly “weighted” with other predictors of VT, SWCT, premature ventricular contractions or ventricular pacing. Moreover, the method can be implemented using a non-transitory computer readable medium that when executed causes the one or more processors to perform the method.

Now referring to FIGS. 29 and 52, other aspects of the present invention that are applicable to the apparatus 2900 and the method 5200 will now be described. In one aspect, the signal change in ventricular repolarization further provides the indication whether the wide complex heart beat(s) is due to ventricular pacing or premature ventricular contraction (PVC). In another aspect, the wide complex heart beat(s) comprise a wide complex tachycardia (WCT), the ventricular source comprises a ventricular tachycardia (VT), and the supraventricular source comprises a supraventricular wide complex tachycardia (SWCT). In another aspect, the signal change in ventricular repolarization comprises a VT probability, the wide complex heart beat classification comprises a VT whenever the VT probability is greater than or equal to the predetermined value, and the wide complex heart beat classification comprises a SWCT whenever the VT probability is less than the predetermined value. In another aspect, the one or more processors select the predetermined value from a range of 0% to 100%. In another aspect, the predetermined value comprises about 1%, 10%, 25%, 50%, 75%, 90% or 99%. In another aspect, the one or more processors provide the signal change in ventricular repolarization by providing a “shock” recommendation signal, a “no shock” recommendation signal, or no signal. In another aspect, the wide complex heart beat waveform amplitudes and/or time-voltage areas of ventricular repolarization comprise T wave amplitudes and/or time-voltage areas, the baseline heart beat waveform amplitudes and/or time-voltage areas of ventricular repolarization comprise baseline T wave amplitudes and/or time-voltage areas, and the T wave amplitudes and/or time-voltage areas and baseline T wave amplitudes and/or time-voltage areas are obtained from an electrocardiogram (ECG) signal, a ventricular electrogram (EMG) signal, a vectorcardiogram (VCG) signal, and/or mathematically-synthesized vectorcardiogram (VCG) signal. In another aspect, the wide complex heart beat T wave amplitudes and/or time-voltage areas comprise a plurality of measured T wave amplitudes and/or time-voltage areas of a ECG waveform, a EMG waveform, a VCG waveform, and/or a mathematically-synthesized VCG waveform above and below an isoelectric baseline; and the baseline heart beat T wave amplitudes and/or time-voltage areas comprise a plurality of measured T wave amplitudes and/or time-voltage areas of a baseline ECG waveform, a baseline EMG waveform, a baseline VCG waveform, and/or a mathematically-synthesized VCG waveform above and below the isoelectric baseline.

In another aspect, the one or more processors receive the one or more wide complex heart beat waveform amplitudes and/or time-voltage areas of ventricular repolarization, and one or more baseline heart beat waveform amplitudes and/or time-voltage areas of ventricular repolarization by: receiving a ECG data, a EMG data, a VCG data, and/or a mathematically-synthesized VCG data via the input/output interface or the memory; receiving a baseline ECG data, a baseline EMG data, a baseline VCG data, and/or a baseline mathematically-synthesized VCG data via the input/output interface or the memory; determining the one or more waveform amplitudes and/or time-voltage areas of ventricular repolarization from the ECG data, the EMG data, the VCG data, and/or the mathematically-synthesized VCG data; and determining the one or more baseline waveform amplitudes and/or time-voltage areas of ventricular repolarization from the baseline ECG data, the baseline EMG data, the baseline VCG data, and/or the baseline mathematically-synthesized VCG data. In another aspect, the ECG data, the EMG data, the VCG data, and/or the mathematically-synthesized VCG data is generated or recorded before or after the baseline ECG data, the baseline EMG data, the baseline VCG data, and/or the baseline mathematically-synthesized VCG data. In another aspect, the ECG data, the EMG data, the VCG data, and/or the mathematically-synthesized VCG data is generated or recorded after the baseline ECG data, the baseline EMG data, the baseline VCG data, and/or the baseline mathematically-synthesized VCG data and determining the signal change in ventricular repolarization. In another aspect, the ECG data, the EMG data, the VCG data, and/or the mathematically-synthesized VCG data and the baseline ECG data, the baseline EMG data, the baseline VCG data, and/or the baseline mathematically-synthesized VCG data are generated or recorded using one or more sensors or devices. In another aspect, the one or more sensors or devices comprise a 12-lead ECG device, a continuous ECG telemetry monitor, a stress testing system, an extended monitoring device, a smartphone-enabled ECG medical device, a cardioverter-defibrillator therapy device, a subcutaneous implantable cardioverter defibrillators (S-ICD), an intracardiac or transvenous pacemaker device, an automated external defibrillators (AED), or an automatic implantable cardioverter defibrillator (AICD). In another aspect, the input/output interface, the memory and the one or more processors are integrated into the one or more sensors or devices; or the one or more sensors or devices are integrated into a computing device comprising the input/output interface, the memory and the one or more processors. In another aspect, the one or more processors determine the signal change in ventricular repolarization between the wide complex heart beat waveform amplitudes and/or time-voltage areas of ventricular repolarization and the baseline heart beat waveform amplitudes and/or time-voltage areas of ventricular repolarization by: receiving a wide complex heart beat waveform duration via the input/output interface or the memory; determining a T-wave percent amplitude change (PAC) based on the wide complex heart beat waveform amplitudes of ventricular repolarization and the baseline heart beat waveform amplitudes of ventricular repolarization, and/or a T-wave percent time-voltage area change (PTVAC) based on the wide complex heart beat waveform time-voltage areas of ventricular repolarization and the baseline heart beat waveform time-voltage areas of ventricular repolarization; determining a classification probability based on the wide complex heart beat waveform duration, and the T-wave PAC and/or the T-wave PTVAC; and wherein the signal change in ventricular repolarization comprises the classification probability, and the classification probability comprises a VT probability, a SWCT probability, a ventricular pacing probability, or a premature ventricular contraction probability. In another aspect, determining the classification probability is further determined based one or more additional classification predictors. In another aspect, the T-wave PAC comprises a frontal T-wave PAC and a horizontal T-wave PAC, and the T-wave PTVAC comprises a frontal T-wave PTVAC and a horizontal T-wave PTVAC.

In another aspect, the one or more processors determine the signal change in ventricular repolarization between the wide complex heart beat waveform amplitudes and/or time-voltage areas of ventricular repolarization and the baseline heart beat waveform amplitudes and/or time-voltage areas of ventricular repolarization by: receiving a WCT duration via the input/output interface or the memory; the one or more wide complex heart beat waveform amplitudes and/or time-voltage areas of ventricular repolarization comprise one or more frontal plane WCT positive T wave amplitudes and/or time-voltage areas, one or more horizontal plane WCT positive T wave amplitudes and/or time-voltage areas, one or more frontal plane WCT negative T wave amplitudes and/or time-voltage areas, and one or more horizontal plane WCT negative T wave amplitudes and/or time-voltage areas; the one or more the baseline heart beat waveform amplitudes and/or time-voltage areas of ventricular repolarization comprise one or more frontal plane baseline positive T wave amplitudes and/or time-voltage areas, one or more horizontal plane baseline positive T wave amplitudes and/or time-voltage areas, one or more frontal plane baseline negative T wave amplitudes and/or time-voltage areas, and one or more horizontal baseline negative T wave amplitudes and/or time-voltage areas; determining (1) a frontal T-wave percent amplitude change (PAC) based on the one or more frontal plane WCT positive T wave amplitudes, one or more frontal plane WCT negative T wave amplitudes, one or more frontal plane baseline positive T wave amplitudes, and one or more frontal plane baseline negative T wave amplitudes, and/or (2) a frontal T-wave percent time-voltage area (PTVAC) based on the one or more frontal plane WCT positive T wave time-voltage areas, one or more frontal plane WCT negative T wave time-voltage areas, one or more frontal plane baseline positive T wave time-voltage areas, and one or more frontal plane baseline negative T wave time-voltage areas; determining (1) a horizontal T-wave PAC based on the one or more horizontal plane WCT positive T wave amplitudes, one or more horizontal plane WCT negative T wave amplitudes, one or more horizontal plane baseline positive T wave amplitudes, and one or more horizontal baseline negative T wave amplitudes, and/or (2) a horizontal T-wave PTVAC based on the one or more horizontal plane WCT positive T wave time-voltage areas, one or more horizontal plane WCT negative T wave time-voltage areas, one or more horizontal plane baseline positive T wave time-voltage areas, and one or more horizontal baseline negative T wave time-voltage areas; determining a VT probability using a statistical or machine learning process based on the WCT duration and (1) the frontal T-wave PAC and the horizontal T-wave PAC, and/or (2) the frontal T-wave PTVAC and the horizontal T-wave PTVAC; and wherein the signal change in ventricular repolarization comprises the VT probability. In another aspect, the statistical or machine learning process comprises a linear regression algorithm, a logistic regression model, a linear discriminate analysis algorithm, a Naive Bayes algorithm, a computational model using artificial neural networks, a computational model based on classification or regression trees, a k-nearest neighbors based model, a support vector machine based model, a boosting algorithm, or an ensemble machine learning algorithm.

In another aspect, the input/output interface comprises a remote device, and the remote device is communicably coupled to the one or more processors via one or more networks. In another aspect, the one or more processors provide a recommendation to select or exclude a therapy, medication, diagnostic testing or referral for a patient based on the signal change in ventricular repolarization. In another aspect, apparatus comprises a server computer, a workstation computer, a laptop computer, a mobile communications device, a personal data assistant, or a medical device.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Sandler I A, Marriott H J. The Differential Morphology of Anomalous Ventricular Complexes of Rbbb-Type in Lead V; Ventricular Ectopy Versus Aberration. Circulation. 1965; 31:551-6.
2. Swanick E J, LaCamera F, Jr., Marriott H J. Morphologic features of right ventricular ectopic beats. Am J Cardiol. 1972; 30(8):888-91.
3. Wellens H J, Bar F W, Lie K I. The value of the electrocardiogram in the differential diagnosis of a tachycardia with a widened QRS complex. Am J Med. 1978; 64(1):27-33.
4. Akhtar M, Shenasa M, Jazayeri M, Caceres J, Tchou P J. Wide QRS complex tachycardia. Reappraisal of a common clinical problem. Ann Intern Med. 1988; 109(11):905-12.
5. Kindwall K E, Brown J, Josephson M E. Electrocardiographic criteria for ventricular tachycardia in wide complex left bundle branch block morphology tachycardias. Am J

Cardiol. 1988; 61(15):1279-83.
6. Brugada P, Brugada J, Mont L, Smeets J, Andries E W. A new approach to the differential diagnosis of a regular tachycardia with a wide QRS complex. Circulation. 1991; 83(5): 1649-59.
7. Griffith M J, de Belder M A, Linker N J, Ward D E, Camm A J. Multivariate analysis to simplify the differential diagnosis of broad complex tachycardia. Br Heart J. 1991; 66(2): 166-74.
8. Griffith M J, Garratt C J, Mounsey P, Camm A J. Ventricular tachycardia as default diagnosis in broad complex tachycardia. Lancet. 1994; 343(8894):386-8.
9. Steurer G, Gursoy S, Frey B, Simonis F, Andries E, Kuck K, et al. The differential diagnosis on the electrocardiogram between ventricular tachycardia and preexcited tachycardia. Clin Cardiol. 1994; 17(6):306-8.
10. Lau E W, Pathamanathan R K, Ng G A, Cooper J, Skehan J D, Griffith M J. The Bayesian approach improves the electrocardiographic diagnosis of broad complex tachycardia. Pacing Clin Electrophysiol. 2000; 23(10 Pt 1):1519-26.
11. Vereckei A, Duray G, Szenasi G, Altemose G T, Miller J M. Application of a new algorithm in the differential diagnosis of wide QRS complex tachycardia. Eur Heart J. 2007; 28(5):589-600.
12. Vereckei A, Duray G, Szenasi G, Altemose G T, Miller J M. New algorithm using only lead aVR for differential diagnosis of wide QRS complex tachycardia. Heart Rhythm. 2008; 5(1):89-98.
13. Sasaki K. A New, Simple Algorithm for Diagnosing Wide QRS Complex Tachycardia: Comparison With Brugada, Vereckei and aVR Algorithms. Circulation. 2009; 120(Issue Supplement 18).
14. Pava L F, Perafan P, Badiel M, Arango J J, Mont L, Morillo C A, et al. R-wave peak time at DII: a new criterion for differentiating between wide complex QRS tachycardias. Heart Rhythm. 2010; 7(7):922-6.
15. Jastrzebski M, Sasaki K, Kukla P, Fijorek K, Stec S, Czarnecka D. The ventricular tachycardia score: a novel approach to electrocardiographic diagnosis of ventricular tachycardia. Europace. 2016; 18(4):578-84.
16. Dongas J, Lehmann M H, Mahmud R, Denker S, Soni J, Akhtar M. Value of preexisting bundle branch block in the electrocardiographic differentiation of supraventricular from ventricular origin of wide QRS tachycardia. The American journal of cardiology. 1985; 55(6):717-21.
17. Alberca T, Almendral J, Sanz P, Almazan A, Cantalapiedra J L, Delcan J L. Evaluation of the specificity of morphological electrocardiographic criteria for the differential diagnosis of wide QRS complex tachycardia in patients with intraventricular conduction defects. Circulation. 1997; 96(10):3527-33.
18. Miller J M, Das M K, Yadav A V, Bhakta D, Nair G, Alberte C. Value of the 12-lead ECG in wide QRS tachycardia. Cardiol Clin. 2006; 24(3):439-51, ix-x.
19. Drew B J, Scheinman M M. ECG criteria to distinguish between aberrantly conducted supraventricular tachycardia and ventricular tachycardia: practical aspects for the immediate care setting. Pacing Clin Electrophysiol. 1995; 18(12 Pt 1):2194-208.
20. Datino T, Almendral J, Avila P, Gonzalez-Torrecilla E, Atienza F, Arenal A, et al. Specificity of electrocardiographic criteria for the differential diagnosis of wide QRS complex tachycardia in patients with intraventricular conduction defect. Heart Rhythm. 2013; 10(9):1393-401.
21. Isenhour J L, Craig S, Gibbs M, Littmann L, Rose G, Risch R. Wide-complex tachycardia: continued evaluation of diagnostic criteria. Acad Emerg Med. 2000; 7(7):769-73.
22. Lau E W, Ng G A. Comparison of the performance of three diagnostic algorithms for regular broad complex tachycardia in practical application. Pacing Clin Electrophysiol. 2002; 25(5): 822-7.
23. Baxi R P, Hart K W, Vereckei A, Miller J, Chung S, Chang W, et al. Vereckei criteria used as a diagnostic tool by emergency medicine residents to distinguish between ventricular tachycardia and supra-ventricular tachycardia with aberrancy. J Cardiol. 2012; 59(3):307-12.
24. Jastrzebski M, Kukla P, Czarnecka D, Kawecka-Jaszcz K. Specificity of the wide QRS complex tachycardia algorithms in recipients of cardiac resynchronization therapy. J Electrocardiol. 2012; 45(3):319-26.
25. Jastrzebski M, Kukla P, Czarnecka D, Kawecka-Jaszcz K. Comparison of five electrocardiographic methods for differentiation of wide QRS-complex tachycardias. Europace. 2012; 14(8):1165-71.
26. Szelenyi Z, Duray G, Katona G, Frituz G, Szego E, Kovacs E, et al. Comparison of the “real-life” diagnostic value of two recently published electrocardiogram methods for the differential diagnosis of wide QRS complex tachycardias. Acad Emerg Med. 2013; 20(11): 1121-30.
27. Kaiser E, Darrieux F C, Barbosa S A, Grinberg R, Assis-Carmo A, Sousa JC, et al. Differential diagnosis of wide QRS tachycardias: comparison of two electrocardiographic algorithms. Europace. 2015; 17(9):1422-7.
28. May A, Brenes-Salazar J, Vaidya V, Ternus B, Kebed D, Brady P. Multistep electrocardiographic algorithms demonstrate inferior specificity for identifying ventricular tachycardia when applied by non-cardiologists. Europace [Internet]. 2015; 17((Supplement 3), iii156).
29. Griffith M J, Debelder M A, Linker N J, Ward D E, Camm A J. Difficulties in the Use of Electrocardiographic Criteria for the Differential-Diagnosis of Left-Bundle-Branch Block Pattern Tachycardia in Patients with a Structurally Normal Heart. European Heart Journal. 1992; 13(4):478-83.
30. Herbert M E, Votey S R, Morgan M T, Cameron P, Dziukas L. Failure to agree on the electrocardiographic diagnosis of ventricular tachycardia. Ann Emerg Med. 1996; 27(1):35-8.
31. Ceresnak S R, Liberman L, Avasarala K, Tanel R, Motonaga K S, Dubin A M. Are wide complex tachycardia algorithms applicable in children and patients with congenital heart disease? J Electrocardiol. 2010; 43(6):694-700.
32. Stewart R B, Bardy G H, Greene H L. Wide Complex Tachycardia-Misdiagnosis and Outcome after Emergent Therapy. Annals of Internal Medicine 1986; 104:766-771.
33. Buxton A E, Marchlinski F E, Doherty J U, Flores B, Josephson M E. Hazards of intravenous verapamil for sustained ventricular tachycardia. Am J Cardiol 1987; 59:1107-10.
34. May A M, Brenes-Salazar J A, DeSimone C V et al. Electrocardiogram algorithms used to differentiate wide complex tachycardias demonstrate diagnostic limitations when applied by non-cardiologists. J Electrocardiol 2018; 51:1103-1109.
35. May A M, DeSimone C V, Kashou AH et al. The WCT Formula: A novel algorithm designed to automatically differentiate wide-complex tachycardias. J Electrocardiol 2019; 54:61-68.
36. May A M, DeSimone C V, Kashou AH et al. The Wide Complex Tachycardia Formula: Derivation and validation data. Data Brief 2019; 24:103924.
37. Landis J R, Koch G G. The measurement of observer agreement for categorical data. Biometrics 1977; 33:159-74.

1-106. (canceled)

107. A computerized method of classifying a wide complex heart beat(s) comprising:
providing a computing device having an input/output interface, one or more processors and a memory;
receiving one or more wide complex heart beat amplitudes and/or time-voltage areas of ventricular repolarization, and one or more baseline heart beat amplitudes and/or time-voltage area of ventricular repolarization via the input/output interface or the memory;
determining a signal change in ventricular repolarization between the wide complex heart beat waveform amplitudes and/or time-voltage areas of ventricular repolarization and the baseline heart beat waveform amplitudes and/or time-voltage areas of ventricular repolarization using the one or more processors; and
providing the signal change in ventricular repolarization via the input/output interface, wherein the signal change in ventricular repolarization provides an indication whether the wide complex heart beat(s) is from a ventricular source or a supraventricular source.

108. The method of claim 107, wherein the signal change in ventricular repolarization further provides the indication whether the wide complex heart beat(s) is due to ventricular pacing or premature ventricular contraction (PVC).

109. The method of claim 107, wherein:
the wide complex heart beat(s) comprise a wide complex tachycardia (WCT);
the ventricular source comprises a ventricular tachycardia (VT); and
the supraventricular source comprises a supraventricular wide complex tachycardia (SWCT).

110. The method of claim 107, wherein providing the signal change in ventricular repolarization via the input/output interface comprises:
automatically determining a wide complex heart beat classification for the wide complex heart beat(s) by comparing the signal change in ventricular repolarization to a predetermined value using the one or more processors, wherein the wide complex heart beat classification comprises the ventricular source or the supraventricular source; and
providing the wide complex heart beat classification via the input/output interface.

111. The method of claim 110, wherein:
the signal change in ventricular repolarization comprises a VT probability;
the wide complex heart beat classification comprises a VT whenever the VT probability is greater than or equal to the predetermined value; and
the wide complex heart beat classification comprises a SWCT whenever the VT probability is less than the predetermined value.

112. The method of claim 111, further comprising selecting the predetermined value from a range of 0% to 100%.

113. (canceled)

114. The method of claim 107, wherein providing the signal change in ventricular repolarization comprises providing a “shock” recommendation signal, a “no shock” recommendation signal, or no signal.

115. The method of claim 107, further comprising obtaining the wide complex heart beat waveform amplitudes and/or time-voltage areas of ventricular repolarization and the baseline heart beat waveform amplitudes and/or time-voltage areas of ventricular repolarization from an electrocardiogram (ECG) signal, a ventricular electrogram (EMG) signal, a vectorcardiogram (VCG) signal and/or a mathematically-synthesized VCG signal, wherein the wide complex heart beat waveform amplitudes and/or time-voltage areas of ventricular repolarization comprise T wave amplitudes and/or time-voltage areas, and the baseline heart beat waveform amplitudes and/or time-voltage areas of ventricular repolarization comprise T wave amplitudes and/or time-voltage areas.

116. The method of claim 115, wherein:
the T wave amplitudes and/or time-voltage areas comprise a plurality of measured T wave amplitudes and/or time-voltage areas of a ECG waveform, a EMG waveform, a VCG waveform, and/or a mathematically-synthesized VCG waveform above and below an isoelectric baseline; and
the baseline T wave amplitudes and/or time-voltage areas comprise a plurality of measured T wave amplitudes and/or time-voltage areas of a baseline ECG waveform, a baseline EMG waveform, a baseline VCG waveform, and/or a baseline mathematically-synthesized VCG waveform above and below the isoelectric baseline.

117. The method of claim 107, wherein receiving the one or more wide complex heart beat waveform amplitudes and/or time-voltage areas of ventricular repolarization, and one or more baseline heart beat waveform amplitudes and/or time-voltage areas of ventricular repolarization comprises:
receiving a ECG data, a EMG data, a VCG data, and/or a mathematically-synthesized VCG data via the input/output interface or the memory;
receiving a baseline ECG data, a baseline EMG data, a baseline VCG data, and/or a baseline mathematically-synthesized VCG data via the input/output interface or the memory;
determining the one or more wide complex heart beat waveform amplitudes and/or time-voltage areas of ventricular repolarization from the ECG data, the EMG data, the VCG data, and/or the mathematically-synthesized VCG data using the one or more processors; and
determining the one or more baseline heart beat waveform amplitudes and/or time-voltage areas of ventricular repolarization from the baseline ECG data, the baseline EMG data, the baseline VCG data, and/or the mathematically-synthesized VCG data using the one or more processors.

118. The method of claim 117, wherein the ECG data, the EMG data, the VCG data, and/or the mathematically-synthesized VCG data is generated or recorded before or after the baseline ECG data, the baseline EMG data, the baseline VCG data, and/or the baseline mathematically-synthesized VCG data.

119. The method of claim 117, wherein the ECG data, the EMG data, the VCG data, and/or mathematically-synthesized VCG data is generated or recorded after the baseline ECG data, the baseline EMG data, the baseline VCG data, and/or the mathematically-synthesized VCG data and determining the signal change.

120. The method of claim 117, further comprising generating or recording the ECG data, the EMG data, the VCG data, and/or the mathematically-synthesized VCG data and the baseline ECG data, the baseline EMG data, the baseline VCG data, and/or the baseline mathematically-synthesized VCG data using one or more sensors or devices.

121-122. (canceled)

123. The method of claim 107, wherein determining the signal change in ventricular repolarization between the wide complex heart beat waveform amplitudes and/or time-voltage areas of ventricular repolarization and the baseline heart beat waveform amplitudes and/or time-voltage areas of ventricular repolarization comprises:
receiving a wide complex heart beat waveform duration via the input/output interface or the memory;
determining, using the one or more processors, a T-wave percent amplitude change (PAC) based on the wide complex heart beat waveform amplitudes of ventricular repolarization and the baseline heart beat waveform amplitudes of ventricular repolarization, and/or a T-wave percent time-voltage area change (PTVAC) based on the wide complex heart beat waveform time-voltage areas of ventricular repolarization and the baseline heart beat waveform time-voltage areas of ventricular repolarization;
determining a classification probability based on the wide complex heart beat waveform duration, and the T-wave PAC and/or the T-wave PTVAC using the one or more processors; and
wherein the signal change in ventricular repolarization comprises the classification probability, and the classification probability comprises a VT probability, a SWCT probability, a ventricular pacing probability, or a premature ventricular contraction probability.

124. The method of claim 123, wherein determining the classification probability is further determined based one or more additional classification predictors.

125. The method of claim 123, wherein:
the T-wave PAC comprises a frontal T-wave PAC and a horizontal T-wave PAC; and
the T-wave PTVAC comprises a frontal T-wave PTVAC and a horizontal T-wave PTVAC.

126. The method of claim 107, wherein determining the signal change in ventricular repolarization between the wide complex heart beat waveform amplitudes and/or time-voltage areas of ventricular repolarization and the baseline heart beat waveform amplitudes and/or time-voltage areas of ventricular repolarization comprises:
receiving a WCT duration via the input/output interface or the memory;
the one or more wide complex heart beat waveform amplitudes and/or time-voltage areas of ventricular repolarization comprise one or more frontal plane WCT positive T wave amplitudes and/or time-voltage areas, one or more horizontal plane WCT positive T wave amplitudes and/or time-voltage areas, one or more frontal plane WCT negative T wave amplitudes and/or time-voltage areas, and one or more horizontal plane WCT negative T wave amplitudes and/or time-voltage areas;
the one or more the baseline heart beat waveform amplitudes and/or time-voltage areas of ventricular repolarization comprise one or more frontal plane baseline positive T wave amplitudes and/or time-voltage areas, one or more horizontal plane baseline positive T wave amplitudes and/or time-voltage areas, one or more frontal plane baseline negative T wave amplitudes and/or time-voltage areas, and one or more horizontal baseline negative T wave amplitudes and/or time-voltage areas;
determining (1) a frontal T-wave percent amplitude change (PAC) based on the one or more frontal plane WCT positive T wave amplitudes, one or more frontal plane WCT negative T wave amplitudes, one or more frontal plane baseline positive T wave amplitudes, and one or more frontal plane baseline negative T wave amplitudes, and/or (2) a frontal T-wave percent time-voltage area (PTVAC) based on the one or more frontal plane WCT positive T wave time-voltage areas, one or more frontal plane WCT negative T wave time-voltage areas, one or more frontal plane baseline positive T wave time-voltage areas, and one or more frontal plane baseline negative T wave time-voltage areas;
determining (1) a horizontal T-wave PAC based on the one or more horizontal plane WCT positive T wave amplitudes, one or more horizontal plane WCT negative T wave amplitudes, one or more horizontal plane baseline positive T wave amplitudes, and one or more horizontal baseline negative T wave amplitudes, and/or (2) a horizontal T-wave PTVAC based on the one or more horizontal plane WCT positive T wave time-voltage areas, one or more horizontal plane WCT negative T wave time-voltage areas, one or more horizontal plane baseline positive T wave time-voltage areas, and one or more horizontal baseline negative T wave time-voltage areas;
determining a VT probability using a statistical or machine learning process based on the WCT duration and (1) the frontal PAC and the horizontal PAC, and/or (2) the frontal PTVAC and the horizontal PTVAC; and
wherein the signal change in ventricular repolarization comprises the VT probability.

127. The method of claim 126, wherein the statistical or machine learning process comprises a linear regression algorithm, a logistic regression model, a linear discriminate analysis algorithm, a Naive Bayes algorithm, a computational model using artificial neural networks, a computational model based on classification or regression trees, a k-nearest neighbors based model, a support vector machine based model, a boosting algorithm, or an ensemble machine learning algorithm.

128. (canceled)

129. The method of claim 107, further comprising providing a recommendation to select or exclude a therapy, medication, diagnostic testing or referral for a patient based on the signal change in ventricular repolarization.

130. (canceled)

131. An apparatus for classifying a wide complex heart beat(s) comprising:
an input/output interface;
a memory; and
one or more processors communicably coupled to the input/output interface and the memory, wherein the one or more processors:
receive one or more wide complex heart beat waveform amplitudes and/or time-voltage areas of ventricular repolarization, and one or more baseline heart beat waveform amplitudes and/or time-voltage areas of ventricular repolarization via the input/output interface or the memory,
determine a signal change in ventricular repolarization between the wide complex heart beat waveform amplitudes and/or time-voltage areas of ventricular repolarization and the baseline heart beat waveform amplitudes and/or time-voltage areas of ventricular repolarization using the one or more processors, and
provide the signal change in ventricular repolarization via the input/output interface, wherein the signal change provides an indication whether the wide complex heart beat(s) is from a ventricular source or a supraventricular source.

132-154. (canceled)
